SEQUENCE LISTING (36 sequences)

SEQ ID NO: 1 - protein, 122 aa, Artificial sequence
  Synthetic 69A7 VH amino acid sequence
QVQLQESGPGLVKPSETLSLTCTVSGGSVSSDYYYWSWIRQPPGKGLEWLGYIYYSGSTNYNPSLKSRVTISVDTSKNQFSLKLRSVTTADTAVYYCARGDGDYGGNCFDYWGQGTLVTVSS

SEQ ID NO: 2 - protein, 107 aa, Artificial Sequence
  Synthetic 69A7 VL amino acid sequence
EIVLTQSPATLSLSPGERATLSCRASQSVSSYLAWYQQKPGQAPRLLIFDASNRATGIPARFSGSGSGTDFTLTISSLEPEDFAVYYCQQRSNWPLTFGGGTKVEIK

SEQ ID NO: 3 - protein, 122 aa, Artificial Sequence
  Synthetic 2A4 VH amino acid sequence
QVQLQESGPGLVKPSETLSLTCTVSGGSVSSDYYYWSWIRQPPGKGLEWLGYIYYSGSTNYNPSLKSRVTISVDTSKNQFSLKLRSVTTADTAVYYCARGDGDYGGNVFPYWGQGTLVTVSS

SEQ ID NO: 4 - protein, 107 aa, Artificial Sequence; identical to SEQ ID NO: 2
  Synthetic 2A4 VL amino acid sequence

SEQ ID NO: 5 - protein, 122 aa, Artificial Sequence
  Synthetic 1H8 VH amino acid sequence
QVQLQESGPGLVKPSETLSLTCTVSGGSVSSDYYYWSWIRQPPGKGLEWLGYIYYSGSTNYNPSLKSRVTISVDTSKNQFSLKLRSVTTADTAVYYCARGDGDFMGVCFDYWGQGTLVTVSS

SEQ ID NO: 6 - protein, 107 aa, Artificial Sequence; identical to SEQ ID NO: 2
  Synthetic 1H8 VL amino acid sequence

SEQ ID NO: 7 - protein, 122 aa, Artificial Sequence
  Synthetic 2E7 VH amino acid sequence
QVQLQESGPGLVKPSETLSLTCTVSGGSVSSDYYYWSWIRQPPGKGLEWLGYIYYSGSTNYNPSLKSRVTISVDTSKNQFSLKLRSVTTADTAVYYCARGDGDFLGVCFDYWGQGTLVTVSS

SEQ ID NO: 8 - protein, 107 aa, Artificial Sequence; identical to SEQ ID NO: 2
  Synthetic 2E7 VL amino acid sequence

SEQ ID NO: 9 - protein, 122 aa, Artificial Sequence; identical to SEQ ID NO: 1
  Synthetic 2D2 VH amino acid sequence

SEQ ID NO: 10 - protein, 107 aa, Artificial Sequence
  Synthetic 2D2 VL amino acid sequence
EIVLTQSPATLSLSPGERATLSCRASQSVSSYLAWYQQKPGQAPRLLIFDASNRATGIPARFSGSGSGTDFTLTISSLEPEDFAVYYCQQRLKFPLTFGGGTKVEIK

SEQ ID NO: 11 - protein, 122 aa, Artificial Sequence
  Synthetic 1A4 VH amino acid sequence
QVQLQESGPGLVKPSETLSLTCTVSGGSVYSGYYYWSWIRQPPGKGLEWLGYFSLSGSTNYNPSLKSRVTISVDTSKNQFSLKLRSVTTADTAVYYCARGDGDYGGNCFDYWGQGTLVTVSS

SEQ ID NO: 12 - protein, 107 aa, Artificial Sequence; identical to SEQ ID NO: 2
  Synthetic 1A4 VL amino acid sequence

SEQ ID NO: 13 - protein, 12 aa, Artificial Sequence
  Synthetic 2A4 HCDR3 amino acid sequence
GDGDYGGNVFPY

SEQ ID NO: 14 - protein, 12 aa, Artificial Sequence
  Synthetic 1H8 HCDR3 amino acid sequence
GDGDFMGVCFDY

SEQ ID NO: 15 - protein, 12 aa, Artificial Sequence
  Synthetic 2E7 HCDR3 amino acid sequence
GDGDFLGVCFDY

SEQ ID NO: 16 - protein, 8 aa, Artificial Sequence
  Synthetic 1A4 HCDR1 amino acid sequence
YSGYYYWS

SEQ ID NO: 17 - protein, 16 aa, Artificial Sequence
  Synthetic 1A4 HCDR2 amino acid sequence
YFSLSGSTNYNPSLKS

SEQ ID NO: 18 - protein, 9 aa, Artificial Sequence
  Synthetic 2D2 LCDR3
QQRLKFPLT

SEQ ID NO: 19 - protein, 118 aa, Artificial Sequence
  Synthetic h1F6 VH amino acid sequence
QVQLVQSGAEVKKPGASVKVSCKASGYTFTNYGMNWVRQAPGQGLKWMGWINTYTGEPTYADAFKGRVTMTRDTSISTAYMELSRLRSDDTAVYYCARDYGDYGMDYWGQGTTVTVSS

SEQ ID NO: 20 - protein, 111 aa, Artificial Sequence
  Synthetic h1F6 VL amino acid sequence
DIVMTQSPDSLAVSLGERATINCRASKSVSTSGYSFMHWYQQKPGQPPKLLIYLASNLESGVPDRFSGSGSGTDFTLTISSLQAEDVAVYYCQHSREVPWTFGQGTKVEIK

SEQ ID NO: 21 - protein, 8 aa, Artificial Sequence
  Synthetic HCDR1 amino acid sequence
SSDYYYWS

SEQ ID NO: 22 - protein, 16 aa, Artificial Sequence
  Synthetic HCDR2 amino acid sequence
YIYYSGSTNYNPSLKS

SEQ ID NO: 23 - protein, 12 aa, Artificial Sequence
  Synthetic HCDR3 amino acid sequence
GDGDYGGNCFDY

SEQ ID NO: 24 - protein, 11 aa, Artificial Sequence
  Synthetic LCDR1 amino acid sequence
RASQSVSSYLA

SEQ ID NO: 25 - protein, 7 aa, Artificial Sequence
  Synthetic LCDR2 amino acid sequence
DASNRAT

SEQ ID NO: 26 - protein, 9 aa, Artificial Sequence
  Synthetic LCDR3 amino acid sequence
QQRSNWPLT

SEQ ID NO: 27 - protein, 5 aa, Artificial Sequence
  Synthetic linker
GGGGS

SEQ ID NO: 28 - protein, 330 aa, Artificial Sequence [sequence omitted]

SEQ ID NO: 29 - protein, 107 aa, Artificial Sequence [sequence omitted]

SEQ ID NO: 30 - protein, 6 aa, Artificial Sequence
  Synthetic hexa-histidine
HHHHHH

SEQ ID NO: 31 - DNA, 9 bp, Artificial Sequence
  Synthetic sequence
gccgccacc

SEQ ID NO: 32 - protein, 19 aa, Artificial Sequence
  Synthetic sequence
MGWSCIILFLVATATGVHS

SEQ ID NO: 33 - protein, 5 aa, Artificial Sequence
  Synthetic sequence
  misc_feature (3)..(3): Xaa can be any naturally occurring amino acid
LPXTG

SEQ ID NO: 34 - protein, 4 aa, Artificial Sequence
  Synthetic linker sequence
  mod_res 1: (Succinimid-3-yl-N)-(CH2)n-C(=O)
  mod_res 4: NH-CH2-O-CH2-(C=O)-
GGFG

SEQ ID NO: 35 - protein, 4 aa, Artificial sequence
  Synthetic linker sequence
GGFG

SEQ ID NO: 36 - protein, 4 aa, Artificial sequence
  Synthetic linker sequence
ALAL

(19) United States
(12) Patent Application Publication
(10) Pub. No.: US 2025/0066490 A1
(43) Pub. Date: Feb. 27, 2025

(54) ANTI-CD70 ANTIBODIES, CONJUGATES THEREOF AND METHODS OF USING THE SAME

(71) Applicant: ProfoundBio US Co., Woodinville, WA (US)

(72) Inventors: Baiteng ZHAO, Bellevue, WA (US); Lei WANG, Suzhou, Jiangsu (CN)

(21) Appl. No.: 18/556,240

(22) PCT Filed: Apr. 22, 2022
(86) PCT No.: PCT/US2022/025966

§ 371 Date: Oct. 19, 2023

(30) Foreign Application Priority Data
Apr. 23, 2021 (WO) PCT/CN2021/089164

(51) Int. Cl. C07K 16/28; A61K 39/00; A61K 47/68; A61P 35/00

(52) U.S. Cl. CPC C07K 16/2875; A61K 47/68031; A61K 47/6849; A61P 35/00; A61K 2039/505; C07K 2317/21; C07K 2317/622; C07K 2317/73; C07K 2317/77; C07K 2317/92

(57) ABSTRACT

The present invention provides CD70 antibodies, antigen binding portions thereof, other binding agents and CD70 conjugates thereof for use in the treatment of cancer and autoimmune disease.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 760270_40101WO_SEQUENCE_LISTING.txt. The text file is 23.6 KB, was created on Apr. 20, 2022, and is being submitted electronically via EFS-Web.

### BACKGROUND

CD70 is member of the tumor necrosis factor (TNF) family of cell membrane-bound and secreted molecules that are expressed by a variety of normal and malignant cell types. CD70 is a transmembrane type II protein with its carboxyl terminus exposed to the outside of cells and its amino terminus found in the cytosolic side of the plasma membrane (Bowman et al., 1994, J. Immunol. 152:1756-61; Goodwin et al, 1993, Cell 73:447-56). Human CD70 contains a 20 amino acid cytoplasmic domain, an 18 amino acid transmembrane domain, and a 155 amino acid extracellular domain with two potential N-linked glycosylation sites (Bowman et al, supra; Goodwin et al, supra). Based on its homology to TNF-alpha and TNF-beta, a trimeric structure is predicted for CD70 (Petsch et al, 1995, Mol. Immunol. 32:761-72).

CD70 has limited expression on normal tissues in humans. This makes CD70 an attractive target for cancer therapies. CD70 expression has been identified on a number of cancers, including renal cell carcer, colon cancer, ovarian cancer, pancreatic cancer, certain types of Non-Hodgkin lymphoma and multiple myeloma. Although CD70 is present on a variety of types of cancer, clinical trials with CD70 antibodies and CD70 antibody drug conjugates have met with limited success. The present invention solves this and other needs.

### SUMMARY OF THE INVENTION

Provided herein are CD70 antibodies, antigen binding portions thereof and other binding agents as well as conjugates of such antibodies, antigen binding portions and other binding agents. Also provided are methods of using the CD70 antibodies, antigen binding portions and other binding agents and conjugates thereof for the treatment of cancer and other diseases. The invention disclosed herein is based in part on CD70 antibodies, antigen-binding portions thereof and other binding agents, as well as conjugates thereof, that specifically bind to CD70 and that exhibit improved properties. CD70 is an important and advantageous therapeutic target for the treatment of certain cancers. The CD70 antibodies, antigen binding portions thereof, other binding agents and conjugates thereof provide compositions and methods based on the use of such antibodies, antigen binding portions and related binding agents, and conjugates thereof, in the treatment of CD70+ cancers and other diseases.

In some embodiments, provided is a binding agent comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having amino acids sequences selected from the sets of amino acid sequences set forth in the group consisting of: SEQ ID NO:21, SEQ ID NO:22, SEQ ID NO:13, SEQ ID NO:24, SEQ ID NO:25 and SEQ ID NO:26, respectively; SEQ ID NO:21, SEQ ID NO:22, SEQ ID NO:14, SEQ ID NO:24, SEQ ID NO:25 and SEQ ID NO:26, respectively; SEQ ID NO:21, SEQ ID NO:22, SEQ ID NO:15, SEQ ID NO:24, SEQ ID NO:25 and SEQ ID NO:26, respectively; SEQ ID NO:21, SEQ ID NO:22, SEQ ID NO:23, SEQ ID NO:24, SEQ ID NO:25 and SEQ ID NO:18, respectively; and SEQ ID NO:16, SEQ ID NO:17, SEQ ID NO:23, SEQ ID NO:24, SEQ ID NO:25 and SEQ ID NO:26; respectively.

In some embodiments, the VH and VL regions have amino acid sequences that are selected from the pairs of amino acid sequences set forth in the group consisting of: SEQ ID NO:3 and SEQ ID NO:4; SEQ ID NO:5 and SEQ ID NO:6; SEQ ID NO:7 and SEQ ID NO:8; SEQ ID NO:9 and SEQ ID NO:10; and SEQ ID NO:11 and SEQ ID NO:12; respectively. In some embodiments, VH and VL regions have amino acid sequences that are selected from the pairs of amino acid sequences set forth in the group consisting of: SEQ ID NO:3 and SEQ ID NO:4; SEQ ID NO:5 and SEQ ID NO:6; SEQ ID NO:7 and SEQ ID NO:8; SEQ ID NO:9 and SEQ ID NO:10; and SEQ ID NO:11 and SEQ ID NO:12; respectively, wherein the heavy and light chain framework regions are optionally modified with from 1 to 8 amino acid substitutions, deletions or insertions in the framework regions. In some embodiments, HCDR1, HCDR2 and HCDR3 and LCDR1, LCDR2 and LCDR3 have the amino acid sequences set forth in SEQ ID NO:21, SEQ ID NO:22, and SEQ ID NO:15, and SEQ ID NO:24, SEQ ID NO:25 and SEQ ID NO:26, respectively.

In some embodiments, wherein the framework regions of the VH and VL are human framework regions. In some embodiments, the binding agent is an antibody or an antigen-binding portion thereof. In some embodiments, the binding agent is a monoclonal antibody, a Fab, a Fab′, an F(ab′), an Fv, a disulfide linked Fc, a scFv, a single domain antibody, a diabody, a bi-specific antibody, or a multi-specific antibody.

In some embodiments, the binding agent has a heavy chain variable region further comprising a heavy chain constant region. In some embodiments, the heavy chain constant region is of the IgG isotype. In some embodiments, the heavy chain constant region is an IgG1 constant region. In some embodiments, the heavy chain constant region is an IgG4 constant region. In some embodiments, the IgG1 constant region has the amino acid sequence set forth in SEQ ID NO:28. In some embodiments, the binding agent has a light chain variable region further comprising a light chain constant region. In some embodiments, the light chain constant region is of the kappa isotype. In some embodiments, the light chain constant region has the amino acid sequence set forth in SEQ ID NO:29. In some embodiments, the heavy chain constant region further comprises at least amino acid modification that decreases binding affinity to human FcgammaRIII.

In some embodiments, the binding agent is mono-specific. In some embodiments, the binding agent is bivalent. In some embodiments, the binding agent is bispecific.

In some embodiments, provided is a pharmaceutical composition comprising any of the binding agents as described herein and a pharmaceutically acceptable carrier.

In some embodiments, provided is a nucleic acid encoding any of the binding agents as described herein. In some embodiments, provided in a vector comprising a nucleic acid encoding any of the binding agents as described herein. In some embodiments, provided is a cell line comprising a vector comprising a nucleic acid encoding any of the binding agents as described herein.

In some embodiments, provided is a conjugate comprising any of the binding agents described herein, at least one linker attached to the binding agent; and at least one drug attached to each linker. In some embodiments, each drug is selected from a cytotoxic agent, an immunomodulatory agents, a nucleic acid, a growth inhibitory agent, a PROTAC, a toxin and a radioactive isotopes. In some embodiments, each linker is attached to the binding agent via an interchain disulfide residue, a lysine residue, an engineered cysteine residue, a glycan, a modified glycan, an N-terminal residue of the binding agent or a polyhistidine residue attached to the binding agent. In some embodiments, the average drug loading of the conjugate is from about 1 to about 8, about 2, about 4, about 6, about 8, about 10, about 12, about 14, about 16, about 3 to about 5, about 6 to about 8, or about 8 to about 16.

In some embodiments, the drug is a cytotoxic agent. In some embodiments, the cytotoxic agent is selected from the group consisting of an auristatin, a maytansinoid, a camptothecin, a duocarmycin, or a calicheamicin. In some embodiments, the cytotoxic agent is an auristatin. In some embodiments, the cytotoxic agent is MMAE or MMAF. In some embodiments, the cytotoxic agent is a camptothecin. In some embodiments, the cytotoxic agent is exatecan. In some embodiments, the cytotoxic agent is SN-38. In some embodiments, the cytotoxic agent is a calicheamicin. In some embodiments, the cytotoxic agent is a maytansinoid. In some embodiments, the maytansinoid is maytansine, maytansinol or a maytansine analog in DM1, DM3 and DM4, and ansamatocin-2.

In some embodiments, the linker is a cleavable linker. In some embodiments, the linker comprises mc-VC-PAB, CL2, CL2A or (Succinimid-3-yl-N)—(CH2)n-C(═O)-Gly-Gly-Phe-Gly-NH—CH2-O—CH2-(C═O)— (SEQ ID NO: 34), wherein n=1 to 5. In some embodiments, the linker comprises mc-VC-PAB. In some embodiments, the linker comprises CL2A. In some embodiments, the linker comprises CL2. In some embodiments, the linker comprises (Succinimid-3-yl-N)—(CH2)n-C(═O)-Gly-Gly-Phe-Gly-NH—CH2-O—CH2-(C═O)— (SEQ ID NO: 34). In some embodiments, the linker is attached to at least one molecule of exatecan.

In some embodiments, the drug is an immune modulatory agent. In some embodiments, the immune modulatory agent is selected from the group consisting of a TRL7 agonist, a TLR8 agonist, a STING agonist, or a RIG-1 agonist. In some embodiments, the immune modulatory agent is an TLR7 agonist. In some embodiments, the TLR7 agonist is an imidazoquinoline, an imidazoquinoline amine, a thiazoquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine, heteroarothiadiazide-2,2-dioxide, a benzonaphthyridine, a guanosine analog, an adenosine analog, a thymidine homopolymer, ssRNA, CpG-A, PolyG10, and PolyG3. In some embodiments, the immune modulatory agent is a TLR8 agonist. In some embodiments, the TLR8 agonist is selected from an imidazoquinoline, a thiazoloquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine or a ssRNA.

In some embodiments, the immune modulatory agent is a STING agonist. In some embodiments, the immune modulatory agent is a RIG-1 agonist. In some embodiments, the RIG-1 agonist is selected from KIN1148, SB-9200, KIN700, KIN600, KIN500, KIN100, KIN101, KIN400 and KIN2000. In some embodiments, linker is selected from the group consisting of mc-VC-PAB, CL2, CL2A and (Succinimid-3-yl-N)—(CH2)n-C(═O)-Gly-Gly-Phe-Gly-NH—CH2-O—CH2-(C═O)— (SEQ ID NO: 34), wherein n=1 to 5.

In some embodiments, provided is a pharmaceutical composition comprising any of the conjugates described herein and a pharmaceutically acceptable carrier.

In some embodiments, provided is method of treating a CD70+ cancer, comprising administering to a subject in need thereof a therapeutically effective amount of any of the binding agents described herein, any of the conjugates described herein or any of the pharmaceutical compositions described herein. In some embodiments, the CD70+ cancer is a solid tumor or a hematologic malignancy. In some embodiments, the CD70+ cancer is selected from hepatocellular cancer, colorectal cancer, pancreatic cancer, ovarian cancer, indolent Non-Hodgkin's Lymphoma, Non-Hodgkin's Lymphoma, cancers of the B-cell lineage, multiple myeloma, renal cell cancers, nasopharyngeal cancers, thymic cancers and gliomas. In some embodiments, the CD70 cancer is a solid tumor.

In some embodiments, the method further comprises administering an immunotherapy to the subject. In some embodiments, the immunotherapy comprises a checkpoint inhibitor. In some embodiments, the checkpoint inhibitor is selected from an antibody that specifically binds to human PD-1, human PD-L1, or human CTLA4. In some embodiments, the checkpoint inhibitor is pembrolizumab, nivolumab, cemiplimab or ipilimumab. In some embodiments, the method further comprises administering chemotherapy to the subject.

In some embodiments, the methods of treating cancer comprise administering any of the conjugates described herein or any of the pharmaceutical compositions described herein. In some embodiments, the binding agent, conjugate or pharmaceutical composition is administered intravenously. In some embodiments, the binding agent, conjugate or pharmaceutical composition is administered in a dose of about 0.1 mg/kg to about 12 mg/kg.

In some embodiments, a treatment outcome of the subject is improved. In some embodiments, the improved treatment outcome is an objective response selected from stable disease, a partial response or a complete response. In some embodiments, the improved treatment outcome is reduced tumor burden. In some embodiments, the improved treatment outcome is progression-free survival or disease-free survival.

In some embodiments, provided is the use of any of the binding agents described herein or any of the pharmaceutical compositions described herein for the treatment of CD70+ cancer in a subject. In some embodiments, provided is the use of any of the conjugates described herein or any of the pharmaceutical compositions described herein for the treatment of CD70+ cancer in a subject.

In some embodiments, provided herein is a method of treating an autoimmune disease, comprising administering to a subject in need thereof a therapeutically effective amount of any of the binding agents described herein, any of the conjugates described herein or any of the pharmaceutical compositions described herein. In some embodiments, the autoimmune disease is rheumatoid arthritis, multiple sclerosis, or systemic lupus erythematosus. In some embodiments, the methods further comprise administering an immunosuppressive therapy to the subject. In some embodiments, method comprises administering any of the conjugates described herein or any of the pharmaceutical compositions described herein.

In some embodiments, the binding agent, conjugate or pharmaceutical composition is administered intravenously. In some embodiments, the binding agent, conjugate or pharmaceutical composition is administered in a dose of about 0.1 mg/kg to about 12 mg/kg. In some embodiments, a treatment outcome of the subject is improved. In some embodiments, the improved treatment outcome is a reduction in disease progression or alleviation of disease severity.

In some embodiments, provided is the use of any of the binding agents described herein or any of the pharmaceutical compositions described herein for the treatment of an autoimmune disease in a subject. In some embodiments, provided is the use of any of the conjugates described herein or any of the pharmaceutical compositions described herein for the treatment of an autoimmune disease in a subject.

These and other aspects of the present invention may be more fully understood by reference to the following detailed description, non-limiting examples of specific embodiments and the appended drawings.

### FIGURES

FIG. 1. Comparison of the relative binding affinities of the lead CD70 scFvs to human CD70 protein.

FIG. 2. Comparison of lead CD70 antibody binding affinity to CD70 protein.

FIG. 3. Cross binding of the lead CD70 antibodies to cynomolgus CD70 protein.

FIG. 4. Comparison of anti-CD70 antibodies binding to 786-O cells.

FIG. 5. Comparison of anti-CD70 antibodies binding to Caki-1 cells.

FIG. 6. Comparison of anti-CD70 antibodies binding to DBTRG-05MG cells.

FIG. 7. Comparison of anti-CD70 antibodies binding to U251 cells.

FIG. 8. Comparison of anti-CD70 antibodies internalization using 786-O cells.

FIG. 9. Comparison of anti-CD70 antibodies internalization using Caki-1 cells.

FIG. 10. Comparison of the cytotoxicity of anti-CD70 conjugates on 786-O renal cell carcinoma cells.

FIG. 11. Binding activity of 2E7 or the isotype control with Raji.

FIG. 12. Binding activity of 2E7 or the isotype control with MCF-7.

FIG. 13. 2E7 internalization in tumor cells.

FIG. 14. 2E7 PK in rat.

FIG. 15. In vitro cell cytotoxicity of 2E7-conjugates on 786-O.

FIG. 16. In vitro cell cytotoxicity of 2E7-conjugates on Raji.

FIG. 17. In vitro cell cytotoxicity of 2E7-conjugates on Caki-1.

FIG. 18. In vitro cell cytotoxicity of 2E7-conjugates on A498.

FIG. 19. Multiple dose study of antitumor activity of 2E7 conjugates with Caki-1.

FIG. 20. Single dose study of antitumor activity of 2E7 conjugates with Caki-1.

FIG. 21. Multiple dose study of antitumor activity of 2E7 conjugates with Raji.

FIG. 22. Single dose study of antitumor activity of 2E7 conjugates with Raji.

### DEFINITIONS

For convenience, certain terms in the specification, examples and claims are defined here. Unless stated otherwise, or implicit from context, the following terms and phrases have the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs.

As used herein and unless otherwise indicated, the terms “a” and “an” are taken to mean “one”, “at least one” or “one or more”. Unless otherwise required by context, singular terms used herein shall include pluralities and plural terms shall include the singular.

Unless the context clearly requires otherwise, throughout the description and the claims, the words “comprise”, “comprising”, and the like are to be construed in an inclusive sense as opposed to an exclusive or exhaustive sense; that is to say, in the sense of “including, but not limited to”.

The terms “decreased,” “reduce,” “reduced”, “reduction”, “decrease,” and “inhibit” are all used herein generally to mean a decrease by a statistically significant amount relative to a reference.

The terms “increased”, “increase” or “enhance” or “activate” are all used herein to generally mean an increase by a statically significant amount relative to a reference.

As used herein, the terms “protein” and “polypeptide” are used interchangeably herein to designate a series of amino acid residues each connected to each other by peptide bonds between the alpha-amino and carboxyl groups of adjacent residues. The terms “protein” and “polypeptide” also refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to an encoded gene product and fragments thereof. Thus, exemplary polypeptides or proteins include gene products, naturally occurring proteins, homologs, orthologs, paralogs, fragments and other equivalents, variants, fragments, and analogs of the foregoing.

CD70 is a cell surface antigen on activated, but not on resting, T and B lymphocytes. It is also referred to as CD27L, Tumor Necrosis Factor (Ligand) Superfamily, Member 7, TNFSF7, Surface Antigen CD70, and Ki-24 Antigen. It is reported to be overexpressed on certain cancers, as further described herein. Human CD70 polypeptides include, but are not limited to, those having the amino acid sequences set forth in UniProt identifiers P32970-1 and P32970-2 and RefSeq NP_001243.1 and NP_001317261.1; these sequences are incorporated by reference herein.

As used herein, an “epitope” refers to the amino acids conventionally bound by an immunoglobulin VH/NL pair, such as the antibodies, antigen binding portions thereof and other binding agents described herein. An epitope can be formed on a polypeptide from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5, about 9, or about 8-10 amino acids in a unique spatial conformation. An epitope defines the minimum binding site for an antibody, antigen binding portions thereof and other binding agent, and thus represents the target of specificity of an antibody, antigen binding portion thereof or other immunoglobulin-based binding agent. In the case of a single domain antibody, an epitope represents the unit of structure bound by a variable domain in isolation.

As used herein, “specifically binds” refers to the ability of a binding agent (e.g., an antibody or antigen binding portion thereof) described herein to bind to a target, such as human CD70, with a KD of 10⁻⁵ M (10000 nM) or less, e.g., 10⁻⁶ M, 10⁻⁷ M, 10⁻⁸ M, 10⁻⁹ M, 10⁻¹⁰ M, 10⁻¹¹ M, 10⁻¹² M, or less. Specific binding can be influenced by, for example, the affinity and avidity of the antibody, antigen binding portion or other binding agent and the concentration of target polypeptide. The person of ordinary skill in the art can determine appropriate conditions under which the antibodies, antigen binding portions and other binding agents described herein selectively bind to CD70 using any suitable methods, such as titration of an antibody or a binding agent in a suitable cell binding assay. A binding agent specifically bound to CD70 is not displaced by a non-similar competitor. In certain embodiments, a CD70 antibody or antigen-binding portion thereof or other binding agent is said to specifically bind to CD70 when it preferentially recognizes its target antigen, CD70, in a complex mixture of proteins and/or macromolecules.

In some embodiments, a CD70 antibody or antigen-binding portion thereof or other binding agent as described herein specifically binds to a CD70 polypeptide with a dissociation constant (KD or K_{D}) of 10⁻⁵ M (10000 nM) or less, e.g., 10⁻⁶ M, 10⁻⁷ M, 10⁻⁸ M, 10⁻⁹ M, 10⁻¹⁰ M, 10⁻¹¹ M, 10⁻¹² M, or less. In some embodiments, a CD70 antibody or antigen-binding portion thereof or other binding agent as described herein specifically binds to a CD70 polypeptide with a dissociation constant (KD) of from about 10⁻⁵ M to 10⁻⁶ M. In some embodiments, a CD70 antibody or antigen-binding portion thereof or other binding agent as described herein specifically binds to a CD70 polypeptide with a dissociation constant (KD) of from about 10⁻⁶ M to 10⁻⁷ M. In some embodiments, a CD70 antibody or antigen-binding portion thereof or other binding agent as described herein specifically binds to a CD70 polypeptide with a dissociation constant (KD) of from about 10⁻⁷ M to 10⁻⁸ M. In some embodiments, a CD70 antibody or antigen-binding portion thereof or other binding agent as described herein specifically binds to a CD70 polypeptide with a dissociation constant (KD) of from about 10⁻⁸ M to 10⁻⁹ M. In some embodiments, a CD70 antibody or antigen-binding portion thereof or other binding agent as described herein specifically binds to a CD70 polypeptide with a dissociation constant (KD) of from about 10⁻⁹ M to 10⁻¹⁰ M. In some embodiments, a CD70 antibody or antigen-binding portion thereof or other binding agent as described herein specifically binds to a CD70 polypeptide with a dissociation constant (KD) of from about 10⁻¹⁰ M to 10⁻¹¹ M. In some embodiments, a CD70 antibody or antigen-binding portion thereof or other binding agent as described herein specifically binds to a CD70 polypeptide with a dissociation constant (KD) of from about 10⁻¹¹ M to 10⁻¹² M. In some embodiments, a CD70 antibody or antigen-binding portion thereof or other binding agent as described herein specifically binds to a CD70 polypeptide with a dissociation constant (KD) of less than 10⁻¹² M.

As used herein, the term “consisting essentially of” refers to those elements required for a given embodiment. The term permits the presence of elements that do not materially affect the basic and novel or functional characteristic(s) of that embodiment.

As used herein, the term “consisting of” refers to compositions, methods, and respective components thereof as described herein, which are exclusive of any element not recited in that description of the embodiment.

Other than in the examples, or where otherwise indicated, all numbers expressing quantities of ingredients or reaction conditions used herein should be understood as modified in all instances by the term “about.” The term “about” when used in connection with percentages can mean+/−1%.

The terms “statistically significant” or “significantly” refer to statistical significance and generally mean a two standard deviation (2SD) difference, above or below a reference value.

Other terms are defined herein within the description of the various aspects of the invention.

### DETAILED DESCRIPTION

Provided herein are CD70 binding antibodies (also referred to as CD70 antibodies) and antigen binding portions thereof and other binding agents that specifically bind to human CD70. Also provided herein are conjugates of the CD70 antibodies and antigen binding portions bound to drugs, such as cytotoxic agents or immune modulatory agents (also referred to as CD70 conjugates). In some embodiments, the CD70 antibodies, antigen binding portions, other binding agents and/or CD70 conjugates specifically bind to and reduce the number of CD70+ cells in a subject. In some embodiments, the CD70 antibodies, antigen binding portions, other binding agents and/or CD70 conjugates specifically bind to and reduce the number of CD70+ cancer cells in a subject. In some embodiments, the CD70 antibodies, antigen binding portions, other binding agents and/or CD70 conjugates specifically bind to and reduce the number of CD70+ cells associated with a disease or condition in a subject, such as an autoimmune disease. In some embodiments, the CD70 antibodies, antigen binding portions, other binding agents and/or CD70 conjugates specifically bind to and reduce the number of CD70+ cells associated with a disease or condition in a subject.

In some embodiments, the CD70 antibodies or antigen binding portions thereof comprise a heavy chain variable region (VH) and a light chain variable region (VL), the VH and VL regions having the amino acid sequences set forth in the pairs of amino acid sequences selected from SEQ ID NO:3 and SEQ ID NO:4, respectively; SEQ ID NO:5 and SEQ ID NO:6, respectively; SEQ ID NO:7 and SEQ ID NO:8, respectively; SEQ ID NO:9 and SEQ ID NO:10, respectively; and SEQ ID NO:11 and SEQ ID NO:12; respectively. In some embodiments, the CD70 antibodies or antigen binding portions thereof comprise a heavy chain variable region (VH) and a light chain variable region (VL), the VH and VL regions having the amino acid sequences set forth in SEQ ID NO:3 and SEQ ID NO:4, respectively. In some embodiments, the CD70 antibodies or antigen binding portions thereof comprise a heavy chain variable region (VH) and a light chain variable region (VL), the VH and VL regions having the amino acid sequences set forth in SEQ ID NO:5 and SEQ ID NO:6, respectively. In some embodiments, the CD70 antibodies or antigen binding portions thereof comprise a heavy chain variable region (VH) and a light chain variable region (VL), the VH and VL regions having the amino acid sequences set forth in SEQ ID NO:7 and SEQ ID NO:8, respectively. In some embodiments, the CD70 antibodies or antigen binding portions thereof comprise a heavy chain variable region (VH) and a light chain variable region (VL), the VH and VL regions having the amino acid sequences set forth in SEQ ID NO:9 and SEQ ID NO:10, respectively. In some embodiments, the CD70 antibodies or antigen binding portions thereof comprise a heavy chain variable region (VH) and a light chain variable region (VL), the VH and VL regions having the amino acid sequences set forth in and SEQ ID NO:11 and SEQ ID NO:12; respectively.

In some embodiments, the CD70 antibodies or antigen binding portions thereof comprise a heavy chain variable region (VH) and a light chain variable region (VL), the VH and VL regions having amino acid sequences set forth in the pairs of amino acid sequences selected from SEQ ID NO:3 and SEQ ID NO:4, respectively; SEQ ID NO:5 and SEQ ID NO:6, respectively; SEQ ID NO:7 and SEQ ID NO:8, respectively; SEQ ID NO:9 and SEQ ID NO:10, respectively; and SEQ ID NO:11 and SEQ ID NO:12; respectively; wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified. In some embodiments, the CD70 antibodies or antigen binding portions thereof comprise a heavy chain variable region (VH) and a light chain variable region (VL), the VH and VL regions having the amino acid sequences set forth in the pairs of amino acid sequences selected from SEQ ID NO:3 and SEQ ID NO:4, respectively; SEQ ID NO:5 and SEQ ID NO:6, respectively; SEQ ID NO:7 and SEQ ID NO:8, respectively; SEQ ID NO:9 and SEQ ID NO:10, respectively; and SEQ ID NO:11 and SEQ ID NO:12; respectively; wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified. The phrase “wherein the CDRs of the heavy or light chain variable regions are not modified” refers to the VH and VL CDRs that do not have amino acid substitutions, deletions or insertions.

In some embodiments, the CD70 antibodies or antigen binding portions thereof comprise a heavy chain variable region (VH) and a light chain variable region (VL), the VH and VL regions having the amino acid sequences set forth in SEQ ID NO:3 and SEQ ID NO:4, respectively; wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified. In some embodiments, the CD70 antibodies or antigen binding portions thereof comprise a heavy chain variable region (VH) and a light chain variable region (VL), the VH and VL regions having the amino acid sequences set forth in SEQ ID NO:3 and SEQ ID NO:4, respectively; wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified.

In some embodiments, the CD70 antibodies or antigen binding portions thereof comprise a heavy chain variable region (VH) and a light chain variable region (VL), the VH and VL regions having the amino acid sequences set forth in SEQ ID NO:5 and SEQ ID NO:6, respectively; wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified. In some embodiments, the CD70 antibodies or antigen binding portions thereof comprise a heavy chain variable region (VH) and a light chain variable region (VL), the VH and VL regions having the amino acid sequences set forth in SEQ ID NO:5 and SEQ ID NO:6, respectively; wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified.

In some embodiments, the CD70 antibodies or antigen binding portions thereof comprise a heavy chain variable region (VH) and a light chain variable region (VL), the VH and VL regions having the amino acid sequences set forth in SEQ ID NO:7 and SEQ ID NO:8, respectively; wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified. In some embodiments, the CD70 antibodies or antigen binding portions thereof comprise a heavy chain variable region (VH) and a light chain variable region (VL), the VH and VL regions having the amino acid sequences set forth in SEQ ID NO:7 and SEQ ID NO:8, respectively; wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified.

In some embodiments, the CD70 antibodies or antigen binding portions thereof comprise a heavy chain variable region (VH) and a light chain variable region (VL), the VH and VL regions having the amino acid sequences set forth in SEQ ID NO:9 and SEQ ID NO:10, respectively; wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified. In some embodiments, the CD70 antibodies or antigen binding portions thereof comprise a heavy chain variable region (VH) and a light chain variable region (VL), the VH and VL regions having the amino acid sequences set forth in SEQ ID NO:9 and SEQ ID NO:10, respectively; wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified.

In some embodiments, the CD70 antibodies or antigen binding portions thereof comprise a heavy chain variable region (VH) and a light chain variable region (VL), the VH and VL regions having the amino acid sequences set forth in and SEQ ID NO: 11 and SEQ ID NO:12; respectively; wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified. In some embodiments, the CD70 antibodies or antigen binding portions thereof comprise a heavy chain variable region (VH) and a light chain variable region (VL), the VH and VL regions having the amino acid sequences set forth in SEQ ID NO: 11 and SEQ ID NO:12; respectively; wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified.

In some embodiments, provided herein is a binding agent comprising a heavy chain variable region (VH) and a light chain variable region (VL), the VH and VL regions having amino acid sequences set forth in the pairs of amino acid sequences selected from SEQ ID NO:3 and SEQ ID NO:4, respectively; SEQ ID NO:5 and SEQ ID NO:6, respectively; SEQ ID NO:7 and SEQ ID NO:8, respectively; SEQ ID NO:9 and SEQ ID NO:10, respectively; and SEQ ID NO:11 and SEQ ID NO:12; respectively; wherein the binding agent specifically binds to CD70. In some embodiments, the binding agent comprises a heavy chain variable region (VH) and a light chain variable region (VL), the VH and VL regions having the amino acid sequences set forth in the pairs of amino acid sequences selected from SEQ ID NO:3 and SEQ ID NO:4, respectively; SEQ ID NO:5 and SEQ ID NO:6, respectively; SEQ ID NO:7 and SEQ ID NO:8, respectively; SEQ ID NO:9 and SEQ ID NO:10, respectively; and SEQ ID NO: 11 and SEQ ID NO:12; respectively; wherein the binding agent specifically binds to CD70 with a higher binding affinity (lower Kd) than that of antibody 69A7. In some embodiments, provided herein is a binding agent comprising a heavy chain variable region (VH) and a light chain variable region (VL), the VH and VL regions having the amino acid sequences set forth in the pairs of amino acid sequences selected from SEQ ID NO:3 and SEQ ID NO:4, respectively; SEQ ID NO:5 and SEQ ID NO:6, respectively; SEQ ID NO:7 and SEQ ID NO:8, respectively; SEQ ID NO:9 and SEQ ID NO:10, respectively; and SEQ ID NO: 11 and SEQ ID NO:12; respectively; wherein the heavy and light chain variable framework regions are optionally modified with from 1 to 8, 1 to 6, 1 to 4 or 1 to 2 conservative amino acid substitutions in the framework regions and wherein the CDRs of the heavy or light chain variable regions are not modified. In some embodiments, provided herein is a binding agent comprising a heavy chain variable region (VH) and a light chain variable region (VL), the VH and VL regions having amino acid sequences set forth in the pairs of amino acid sequences selected from SEQ ID NO:3 and SEQ ID NO:4, respectively; SEQ ID NO:5 and SEQ ID NO:6, respectively; SEQ ID NO:7 and SEQ ID NO:8, respectively; SEQ ID NO:9 and SEQ ID NO:10, respectively; and SEQ ID NO:11 and SEQ ID NO:12; respectively; wherein the heavy and light chain variable framework regions are optionally modified with from 1 to 8, 1 to 6, 1 to 4 or 1 to 2 amino acid substitutions, deletions or insertions in the framework regions and wherein the CDRs of the heavy or light chain variable regions are not modified. As described herein, a binding agent includes a CD70 antibody or antigen binding portion(s) thereof and can optionally include other peptides or polypeptides covalently attached to the CD70 antibody or antigen binding portion thereof. In any of these embodiments, the binding agent specifically binds to CD70.

In some embodiments, provided herein is a binding agent comprising a heavy chain variable region (VH) and a light chain variable region (VL), the VH and VL regions having the amino acid sequences set forth in SEQ ID NO:3 and SEQ ID NO:4, respectively; wherein the binding agent specifically binds to CD70. In some embodiments, the binding agent comprises a heavy chain variable region (VH) and a light chain variable region (VL), the VH and VL regions having amino acid sequences set forth SEQ ID NO:3 and SEQ ID NO:4, respectively; wherein the binding agent specifically binds to CD70 with a higher binding affinity (lower Kd) than that of antibody 69A7. In some embodiments, provided herein is a binding agent comprising a heavy chain variable region (VH) and a light chain variable region (VL), the VH and VL regions having the amino acid sequences set forth in SEQ ID NO:3 and SEQ ID NO:4, respectively; wherein the heavy and light chain variable framework regions are optionally modified with from 1 to 8, 1 to 6, 1 to 4 or 1 to 2 conservative amino acid substitutions in the framework regions and wherein the CDRs of the heavy or light chain variable regions are not modified. In some embodiments, provided herein is a binding agent comprising a heavy chain variable region (VH) and a light chain variable region (VL), the VH and VL regions having the amino acid sequences set forth in SEQ ID NO:3 and SEQ ID NO:4, respectively; wherein the heavy and light chain variable framework regions are optionally modified with from 1 to 8, 1 to 6, 1 to 4 or 1 to 2 amino acid substitutions, deletions or insertions in the framework regions and wherein the CDRs of the heavy or light chain variable regions are not modified.

In some embodiments, provided herein is a binding agent comprising a heavy chain variable region (VH) and a light chain variable region (VL), the VH and VL regions having the amino acid sequences set forth in SEQ ID NO:5 and SEQ ID NO:6, respectively; wherein the binding agent specifically binds to CD70. In some embodiments, the binding agent comprises a heavy chain variable region (VH) and a light chain variable region (VL), the VH and VL regions having the amino acid sequences set forth in SEQ ID NO:5 and SEQ ID NO:6, respectively; wherein the binding agent specifically binds to CD70 with a higher binding affinity (lower Kd) than that of antibody 69A7. In some embodiments, provided herein is a binding agent comprising a heavy chain variable region (VH) and a light chain variable region (VL), the VH and VL regions having the amino acid sequences set forth in SEQ ID NO:5 and SEQ ID NO:6, respectively; wherein the heavy and light chain variable framework regions are optionally modified with from 1 to 8, 1 to 6, 1 to 4 or 1 to 2 conservative amino acid substitutions in the framework regions and wherein the CDRs of the heavy or light chain variable regions are not modified. In some embodiments, provided herein is a binding agent comprising a heavy chain variable region (VH) and a light chain variable region (VL), the VH and VL regions having the amino acid sequences set forth in SEQ ID NO:5 and SEQ ID NO:6, respectively; wherein the heavy and light chain variable framework regions are optionally modified with from 1 to 8, 1 to 6, 1 to 4 or 1 to 2 amino acid substitutions, deletions or insertions in the framework regions and wherein the CDRs of the heavy or light chain variable regions are not modified.

In some embodiments, provided herein is a binding agent comprising a heavy chain variable region (VH) and a light chain variable region (VL), the VH and VL regions having the amino acid sequences set forth in SEQ ID NO:7 and SEQ ID NO:8, respectively; wherein the binding agent specifically binds to CD70. In some embodiments, the binding agent comprises a heavy chain variable region (VH) and a light chain variable region (VL), the VH and VL regions having the amino acid sequences set forth in SEQ ID NO:7 and SEQ ID NO:8, respectively; wherein the binding agent specifically binds to CD70 with a higher binding affinity (lower Kd) than that of antibody 69A7. In some embodiments, provided herein is a binding agent comprising a heavy chain variable region (VH) and a light chain variable region (VL), the VH and VL regions having the amino acid sequences set forth in SEQ ID NO:7 and SEQ ID NO:8, respectively; wherein the heavy and light chain variable framework regions are optionally modified with from 1 to 8, 1 to 6, 1 to 4 or 1 to 2 conservative amino acid substitutions in the framework regions and wherein the CDRs of the heavy or light chain variable regions are not modified. In some embodiments, provided herein is a binding agent comprising a heavy chain variable region (VH) and a light chain variable region (VL), the VH and VL regions having the amino acid sequences set forth in SEQ ID NO:7 and SEQ ID NO:8, respectively; wherein the heavy and light chain variable framework regions are optionally modified with from 1 to 8, 1 to 6, 1 to 4 or 1 to 2 amino acid substitutions, deletions or insertions in the framework regions and wherein the CDRs of the heavy or light chain variable regions are not modified.

In some embodiments, provided herein is a binding agent comprising a heavy chain variable region (VH) and a light chain variable region (VL), the VH and VL regions having the amino acid sequences set forth in SEQ ID NO:9 and SEQ ID NO:10, respectively; wherein the binding agent specifically binds to CD70. In some embodiments, the binding agent comprises a heavy chain variable region (VH) and a light chain variable region (VL), the VH and VL regions having the amino acid sequences set forth in SEQ ID NO:9 and SEQ ID NO:10, respectively; wherein the binding agent specifically binds to CD70 with a higher binding affinity (lower Kd) than antibody 69A7. In some embodiments, provided herein is a binding agent comprising a heavy chain variable region (VH) and a light chain variable region (VL), the VH and VL regions having the amino acid sequences set forth in SEQ ID NO:9 and SEQ ID NO:10, respectively; wherein the heavy and light chain variable framework regions are optionally modified with from 1 to 8, 1 to 6, 1 to 4 or 1 to 2 conservative amino acid substitutions in the framework regions and wherein the CDRs of the heavy or light chain variable regions are not modified. In some embodiments, provided herein is a binding agent comprising a heavy chain variable region (VH) and a light chain variable region (VL), the VH and VL regions having the amino acid sequences set forth in SEQ ID NO:9 and SEQ ID NO:10, respectively; wherein the heavy and light chain variable framework regions are optionally modified with from 1 to 8, 1 to 6, 1 to 4 or 1 to 2 amino acid substitutions, deletions or insertions in the framework regions and wherein the CDRs of the heavy or light chain variable regions are not modified.

In some embodiments, provided herein is a binding agent comprising a heavy chain variable region (VH) and a light chain variable region (VL), the VH and VL regions having the amino acid sequences set forth in SEQ ID NO:11 and SEQ ID NO:12; respectively; wherein the binding agent specifically binds to CD70. In some embodiments, the binding agent comprises a heavy chain variable region (VH) and a light chain variable region (VL), the VH and VL regions having the amino acid sequences set forth in SEQ ID NO: 11 and SEQ ID NO:12; respectively; wherein the binding agent specifically binds to CD70 with a higher binding affinity (lower Kd) than that of antibody 69A7. In some embodiments, provided herein is a binding agent comprising a heavy chain variable region (VH) and a light chain variable region (VL), the VH and VL regions having the amino acid sequences set forth in SEQ ID NO:11 and SEQ ID NO:12; respectively; wherein the heavy and light chain variable framework regions are optionally modified with from 1 to 8, 1 to 6, 1 to 4 or 1 to 2 conservative amino acid substitutions in the framework regions and wherein the CDRs of the heavy or light chain variable regions are not modified. In some embodiments, provided herein is a binding agent comprising a heavy chain variable region (VH) and a light chain variable region (VL), the VH and VL regions having the amino acid sequences set forth in SEQ ID NO:11 and SEQ ID NO:12; respectively; wherein the heavy and light chain variable framework regions are optionally modified with from 1 to 8, 1 to 6, 1 to 4 or 1 to 2 amino acid substitutions, deletions or insertions in the framework regions and wherein the CDRs of the heavy or light chain variable regions are not modified.

In some embodiments, provided is an antibody or antigen binding portion comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having the amino acids sequences set forth in the sets of amino acid sequences selected from (i) SEQ ID NO:21, SEQ ID NO:22, SEQ ID NO:13, SEQ ID NO:24, SEQ ID NO:25 and SEQ ID NO:26, respectively; (ii) SEQ ID NO:21, SEQ ID NO:22, SEQ ID NO:14, SEQ ID NO:24, SEQ ID NO:25 and SEQ ID NO:26, respectively; (iii) SEQ ID NO:21, SEQ ID NO:22, SEQ ID NO:15, SEQ ID NO:24, SEQ ID NO:25 and SEQ ID NO:26, respectively; (iv) SEQ ID NO:21, SEQ ID NO:22, SEQ ID NO:23, SEQ ID NO:24, SEQ ID NO:25 and SEQ ID NO:18, respectively; and (v) SEQ ID NO:16, SEQ ID NO:17, SEQ ID NO:23, SEQ ID NO:24, SEQ ID NO:25 and SEQ ID NO:26; respectively. In some embodiments, each VH and VL region comprises a humanized framework region. In some embodiments, each VH and VL region comprises a human framework region.

In some embodiments, provided is an antibody or antigen binding portion comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having the amino acids sequences set forth in SEQ ID NO:21, SEQ ID NO:22, SEQ ID NO:13, SEQ ID NO:24, SEQ ID NO:25 and SEQ ID NO:26, respectively. In some embodiments, each VH and VL region comprises a humanized framework region. In some embodiments, each VH and VL region comprises a human framework region.

In some embodiments, provided is an antibody or antigen binding portion comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having the amino acids sequences set forth in SEQ ID NO:21, SEQ ID NO:22, SEQ ID NO:14, SEQ ID NO:24, SEQ ID NO:25 and SEQ ID NO:26, respectively. In some embodiments, each VH and VL region comprises a humanized framework region. In some embodiments, each VH and VL region comprises a human framework region.

In some embodiments, provided is an antibody or antigen binding portion comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having the amino acids sequences set forth in SEQ ID NO:21, SEQ ID NO:22, SEQ ID NO:15, SEQ ID NO:24, SEQ ID NO:25 and SEQ ID NO:26, respectively. In some embodiments, each VH and VL region comprises a humanized framework region. In some embodiments, each VH and VL region comprises a human framework region.

In some embodiments, provided is an antibody or antigen binding portion comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having the amino acids sequences set forth in SEQ ID NO:21, SEQ ID NO:22, SEQ ID NO:23, SEQ ID NO:24, SEQ ID NO:25 and SEQ ID NO:18, respectively. In some embodiments, each VH and VL region comprises a humanized framework region. In some embodiments, each VH and VL region comprises a human framework region.

In some embodiments, provided is a binding agent comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having the amino acids sequences set forth in SEQ ID NO:16, SEQ ID NO:17, SEQ ID NO:23, SEQ ID NO:24, SEQ ID NO:25 and SEQ ID NO:26; respectively. In some embodiments, each VH and VL region comprises a humanized framework region. In some embodiments, each VH and VL region comprises a human framework region. In some embodiments, provided is a binding agent comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having the amino acids sequences set forth in the sets of amino acid sequences selected from (i) SEQ ID NO:21, SEQ ID NO:22, SEQ ID NO:13, SEQ ID NO:24, SEQ ID NO:25 and SEQ ID NO:26, respectively; (ii) SEQ ID NO:21, SEQ ID NO:22, SEQ ID NO:14, SEQ ID NO:24, SEQ ID NO:25 and SEQ ID NO:26, respectively; (iii) SEQ ID NO:21, SEQ ID NO:22, SEQ ID NO:15, SEQ ID NO:24, SEQ ID NO:25 and SEQ ID NO:26, respectively; (iv) SEQ ID NO:21, SEQ ID NO:22, SEQ ID NO:23, SEQ ID NO:24, SEQ ID NO:25 and SEQ ID NO:18, respectively; and (v) SEQ ID NO:16, SEQ ID NO:17, SEQ ID NO:23, SEQ ID NO:24, SEQ ID NO:25 and SEQ ID NO:26; respectively. In some embodiments, each VH and VL region comprises a humanized framework region. In some embodiments, each VH and VL region comprises a human framework region.

In some embodiments, provided is a binding agent comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having the amino acids sequences set forth in SEQ ID NO:21, SEQ ID NO:22, SEQ ID NO:13, SEQ ID NO:24, SEQ ID NO:25 and SEQ ID NO:26, respectively. In some embodiments, each VH and VL region comprises a humanized framework region. In some embodiments, each VH and VL region comprises a human framework region.

In some embodiments, provided is a binding agent comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having the amino acids sequences set forth in SEQ ID NO:21, SEQ ID NO:22, SEQ ID NO:14, SEQ ID NO:24, SEQ ID NO:25 and SEQ ID NO:26, respectively. In some embodiments, each VH and VL region comprises a humanized framework region. In some embodiments, each VH and VL region comprises a human framework region.

In some embodiments, provided is a binding agent comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having the amino acids sequences set forth in SEQ ID NO:21, SEQ ID NO:22, SEQ ID NO:15, SEQ ID NO:24, SEQ ID NO:25 and SEQ ID NO:26, respectively. In some embodiments, each VH and VL region comprises a humanized framework region. In some embodiments, each VH and VL region comprises a human framework region.

In some embodiments, provided is a binding agent comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having the amino acids sequences set forth in SEQ ID NO:21, SEQ ID NO:22, SEQ ID NO:23, SEQ ID NO:24, SEQ ID NO:25 and SEQ ID NO:18, respectively. In some embodiments, each VH and VL region comprises a humanized framework region. In some embodiments, each VH and VL region comprises a human framework region.

In some embodiments, provided is a binding agent comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having the amino acids sequences set forth in SEQ ID NO:16, SEQ ID NO:17, SEQ ID NO:23, SEQ ID NO:24, SEQ ID NO:25 and SEQ ID NO:26; respectively. In some embodiments, each VH and VL region comprises a humanized framework region. In some embodiments, each VH and VL region comprises a human framework region.

In some embodiments, the compositions and methods described herein relate to reduction of CD70+ cells in a subject (e.g., reducing the number of CD70+ cells in a cancer or tumor, or CD70+ cells associated with an autoimmune disease or disorder) by a CD70 antibody, antigen binding portion thereof, other binding agent or conjugate thereof in vivo. In some embodiments, the compositions and methods described herein relate to the treatment of CD70+ cancer in a subject by administering a CD70 antibody, antigen binding portion thereof, other binding agent or conjugate thereof. In some embodiments, the compositions and methods described herein relate to the treatment of an autoimmune disorder in a subject by administering a CD70 antibody, antigen binding portion thereof, other binding agent or conjugate thereof. In some embodiments, the compositions and methods described herein relate to the treatment of disease or disorder associated with CD70+ cells in a subject by administering a CD70 antibody, antigen binding portion thereof, other binding agent or conjugate thereof. In any of these embodiments, the methods further include a reduction in the number of CD70+ cells in the subject that are associated with the disease, condition or cancer.

As used herein, the term “antibody” refers to immunoglobulin molecules and immunologically active portions of immunoglobulin molecules, i.e., molecules that contain an antigen binding site(s) that specifically binds to an antigen, e.g., human CD70. The term generally refers to antibodies comprised of two immunoglobulin heavy chain variable regions and two immunoglobulin light chain variable regions including full length antibodies (having heavy and light chain constant regions).

Each heavy chain is composed of a variable region (abbreviated as VH) and a constant region. The heavy chain constant region may include three domains CH1, CH2 and CH3 and optionally a fourth domain, CH4. Each light chain is composed of a variable region (abbreviated as VL) and a constant region. The light chain constant region is a CL domain. The VH and VL regions may be further divided into hypervariable regions referred to as complementarity-determining regions (CDRs) and interspersed with conserved regions referred to as framework regions (FR). Each VH and VL region thus consists of three CDRs and four FRs that are arranged from the N terminus to the C terminus in the following order: FR1, CDR1, FR2, CDR2, FR3, CDR3, and FR4. This structure is well known to those skilled in the art.

As used herein, an “antigen-binding portion” of a CD70 antibody refers to the portions of a CD70 antibody as described herein having the VH and VL sequences of the CD70 antibody or the CDRs of a CD70 antibody and that specifically binds to CD70. Examples of antigen binding portions include a Fab, a Fab′, a F(ab′)₂, a Fv, a scFv, a disulfide linked Fv, a single domain antibody (also referred to as a VHH, VNAR, sdAb, or nanobody) or a diabody (see, e.g., Huston et al., Proc. Natl. Acad. Sci. U.S.A., 85, 5879-5883 (1988) and Bird et al., Science 242, 423-426 (1988), which are incorporated herein by reference). As used herein, the terms Fab, F(ab′)₂ and Fv refer to the following: (i) a Fab fragment, i.e. a monovalent fragment composed of the VL, VH, CL and CH1 domains; (ii) an F(ab′)₂ fragment, i.e. a bivalent fragment comprising two Fab fragments linked to one another in the hinge region via a disulfide bridge; and (iii) an Fv fragment composed of the VL and VH domains, in each case of a CD70 antibody. Although the two domains of the Fv fragment, namely VL and VH, are encoded by separate coding regions, they may further be linked to one another using a synthetic linker, e.g., a poly-G4S amino acid sequence (‘(G₄S)ₙ’ disclosed as SEQ ID NO: 27, wherein n=1 to 5), making it possible to prepare them as a single protein chain in which the VL and VH regions combine in order to form monovalent molecules (known as single chain Fv or scFv). The term “antigen-binding portion” of an antibody is also intended to include such single chain antibodies. Other forms of single chain antibodies such as “diabodies” are likewise included here. Diabodies are bivalent, bispecific antibodies in which VH and VL domains are expressed on a single polypeptide chain, but using a linker connecting the VH and VL domains that is too short for the two domains to be able to combine on the same chain, thereby forcing the VH and VL domains to pair with complementary domains of a different chain (VL and VH, respectively), and to form two antigen-binding sites (see, for example, Holliger, R, et al. (1993) Proc. Natl. Acad. Sci. USA 90:64446448; Poljak, R. J, et al. (1994) Structure 2:1121-1123).

A single-domain antibody is an antibody portion consisting of a single monomeric variable antibody domain. Single domains antibodies can be derived from the variable domain of the antibody heavy chain from camelids (e.g., nanobodies or VHH portions). Furthermore, the term single-domain antibody includes an autonomous human heavy chain variable domain (aVH) or VNAR portions derived from sharks (see, e.g., Hasler et al., Mol. Immunol. 75:28-37, 2016).

Techniques for producing single domain antibodies (e.g., DABs or VHH) are known in the art, as disclosed for example in Cossins et al. (2006, Prot Express Purif 51:253-259) and Li et al. (Immunol. Lett. 188:89-95, 2017). Single domain antibodies may be obtained, for example, from camels, alpacas or llamas by standard immunization techniques. (See, e.g., Muyldermans et al., TIBS 26:230-235, 2001; Yau et al., J Immunol Methods 281:161-75, 2003; and Maass et al., J Immunol Methods 324:13-25, 2007.) A VHH may have potent antigen-binding capacity and can interact with novel epitopes that are inaccessible to conventional VH-VL pairs (see, e.g., Muyldermans et al., 2001). Alpaca serum IgG contains about 50% camelid heavy chain only IgG antibodies (HCAbs) (see, e.g., Maass et al., 2007). Alpacas may be immunized with antigens and VHHs can be isolated that bind to and neutralize a target antigen (see, e.g., Maass et al., 2007). PCR primers that amplify alpaca VHH coding sequences have been identified and may be used to construct alpaca VHH phage display libraries, which can be used for antibody fragment isolation by standard biopanning techniques well known in the art (see, e.g., Maass et al., 2007).

In some embodiments, the CD70 antibodies or antigen binding portions thereof are part of a bispecific or multispecific binding agent. Bispecific and multi-specific antibodies include the following: an scFv1-ScFv2, an ScFv1₂-Fc-scFv2₂, an IgG-scFv, a DVD-Ig, a triomab/quadroma, a two-in-one IgG, a scFv2-Fc, a TandAb, and an scFv-HSA-scFv. In some embodiments, an IgG-scFv is an IgG(H)-scFv, scFv-(H)IgG, IgG(L)-scFv, svFc-(L)IgG, 2scFV-IgG or IgG-2scFv. See, e.g., Brinkmann and Kontermann, MAbs 9(2):182-212 (2017); Wang et al., Antibodies, 2019, 8, 43; Dong et al., 2011, MAbs 3:273-88; Natsume et al., J. Biochem. 140(3):359-368, 2006; Cheal et al., Mol. Cancer Ther. 13(7):1803-1812, 2014; and Bates and Power, Antibodies, 2019, 8, 28.

### Modification of VH and VL Regions

As to the VH and VL amino acid sequences, one of skill will recognize that individual substitutions, deletions or additions (insertions) to a nucleic acid encoding the VH or VL, or amino acids in a polypeptide that alter a single amino acid or a small percentage of amino acids in the encoded sequence is a “conservatively modified variant”, where the alteration results in the substitution of an amino acid with a chemically similar amino acid (a conservative amino acid substitution) and the altered polypeptide retains the ability to specifically bind to CD70.

In some embodiments, a conservatively modified variant of a CD70 antibody or antigen binding portion thereof can have an alteration(s) in the framework regions (i.e., other than in the CDRs), e.g. a conservatively modified variant of a CD70 antibody has the amino acid sequences of the VH and VL CDRs (set forth in sets of amino acid sequences SEQ ID NO:21, SEQ ID NO:22, SEQ ID NO:13, SEQ ID NO:24, SEQ ID NO:25 and SEQ ID NO:26; SEQ ID NO:21, SEQ ID NO:22, SEQ ID NO:14, SEQ ID NO:24, SEQ ID NO:25 and SEQ ID NO:26; SEQ ID NO:21, SEQ ID NO:22, SEQ ID NO:15, SEQ ID NO:24, SEQ ID NO:25 and SEQ ID NO:26; SEQ ID NO:21, SEQ ID NO:22, SEQ ID NO:23, SEQ ID NO:24, SEQ ID NO:25 and SEQ ID NO:18; and SEQ ID NO:16, SEQ ID NO:17, SEQ ID NO:23, SEQ ID NO:24, SEQ ID NO:25 and SEQ ID NO:26) and has at least one conservative amino acid substitution in a framework region (FR). In some embodiments, the VH and VL amino acid sequences collectively have no more than 8 or 6 or 4 or 2 or 1 conservative amino acid substitutions in the FR, as compared to the amino acid sequences of the unmodified VH and VL regions. In some embodiments, the VH and VL amino acid sequences have 8 to 1, 6 to 1, 4 to 1 or 2 to 1 conservative amino acid substitutions in the FR, as compared to the amino acid sequences of the unmodified VH and VL regions. In further aspects of any of these embodiments, a conservatively modified variant of the CD70 antibody, antigen binding portion thereof or other binding agent exhibits specific binding to CD70.

For conservative amino acid substitutions, a given amino acid can be replaced by a residue having similar physiochemical characteristics, e.g., substituting one aliphatic residue for another (such as lie, Val, Leu, or Ala for one another), or substitution of one polar residue for another (such as between Lys and Arg; Glu and Asp; or Gln and Asn). Other such conservative amino acid substitutions, e.g., substitutions of entire regions having similar hydrophobicity characteristics, are well known. Polypeptides comprising conservative amino acid substitutions can be tested in any one of the assays described herein to confirm that a desired activity, e.g. antigen-binding activity and specificity of a native or reference polypeptide is retained, i.e., to CD70.

In some embodiments, a CD70 antibody or antigen binding portion thereof or other binding agent can be further optimized to, for example, decrease potential immunogenicity or optimize other functional property, while maintaining functional activity, for therapy in humans. In some embodiments, the CD70 antibodies or antigen binding portions thereof or other binding agents comprise a heavy chain variable region (VH) and a light chain variable region (VL), the VH and VL regions having amino acid sequences set forth in the pairs of amino acid sequences selected from SEQ ID NO:3 and SEQ ID NO:4, respectively; SEQ ID NO:5 and SEQ ID NO:6, respectively; SEQ ID NO:7 and SEQ ID NO:8, respectively; SEQ ID NO:9 and SEQ ID NO:10, respectively; and SEQ ID NO:11 and SEQ ID NO:12; respectively; wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified. In some embodiments, the CD70 antibodies or antigen binding portions thereof or other binding agents comprise a heavy chain variable region (VH) and a light chain variable region (VL), the VH and VL regions having amino acid sequences set forth in the pairs of amino acid sequences selected from SEQ ID NO:3 and SEQ ID NO:4, respectively; SEQ ID NO:5 and SEQ ID NO:6, respectively; SEQ ID NO:7 and SEQ ID NO:8, respectively; SEQ ID NO:9 and SEQ ID NO:10, respectively; and SEQ ID NO:11 and SEQ ID NO:12; respectively; wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified.

In some embodiments, provided herein is a CD70 antibody or antigen binding portion thereof or other binding agent comprising a heavy chain variable region (VH) and a light chain variable region (VL), the VH and VL regions having amino acid sequences set forth in SEQ ID NO:3 and SEQ ID NO:4, respectively; wherein the heavy and light chain variable framework regions are optionally modified with from 1 to 8, 1 to 6, 1 to 4 or 1 to 2 conservative amino acid substitutions in the framework regions and wherein the CDRs of the heavy or light chain variable regions are not modified. In some embodiments, provided herein is a CD70 antibody or antigen binding portion thereof or other binding agent comprising a heavy chain variable region (VH) and a light chain variable region (VL), the VH and VL regions having amino acid sequences set forth in SEQ ID NO:3 and SEQ ID NO:4, respectively; wherein the heavy and light chain variable framework regions are optionally modified with from 1 to 8, 1 to 6, 1 to 4 or 1 to 2 amino acid substitutions, deletions or insertions in the framework regions and wherein the CDRs of the heavy or light chain variable regions are not modified.

In some embodiments, provided herein is a CD70 antibody or antigen binding portion thereof or other binding agent comprising a heavy chain variable region (VH) and a light chain variable region (VL), the VH and VL regions having amino acid sequences set forth in SEQ ID NO:5 and SEQ ID NO:6, respectively; wherein the heavy and light chain variable framework regions are optionally modified with from 1 to 8, 1 to 6, 1 to 4 or 1 to 2 conservative amino acid substitutions in the framework regions and wherein the CDRs of the heavy or light chain variable regions are not modified. In some embodiments, provided herein is a CD70 antibody or antigen binding portion thereof or other binding agent comprising a heavy chain variable region (VH) and a light chain variable region (VL), the VH and VL regions having amino acid sequences set forth in SEQ ID NO:5 and SEQ ID NO:6, respectively; wherein the heavy and light chain variable framework regions are optionally modified with from 1 to 8, 1 to 6, 1 to 4 or 1 to 2 amino acid substitutions, deletions or insertions in the framework regions and wherein the CDRs of the heavy or light chain variable regions are not modified.

In some embodiments, provided herein is a CD70 antibody or antigen binding portion thereof or other binding agent comprising a heavy chain variable region (VH) and a light chain variable region (VL), the VH and VL regions having amino acid sequences set forth in SEQ ID NO:7 and SEQ ID NO:8, respectively; wherein the heavy and light chain variable framework regions are optionally modified with from 1 to 8, 1 to 6, 1 to 4 or 1 to 2 conservative amino acid substitutions in the framework regions and wherein the CDRs of the heavy or light chain variable regions are not modified. In some embodiments, provided herein is a CD70 antibody or antigen binding portion thereof or other binding agent comprising a heavy chain variable region (VH) and a light chain variable region (VL), the VH and VL regions having amino acid sequences set forth in SEQ ID NO:7 and SEQ ID NO:8, respectively; wherein the heavy and light chain variable framework regions are optionally modified with from 1 to 8, 1 to 6, 1 to 4 or 1 to 2 amino acid substitutions, deletions or insertions in the framework regions and wherein the CDRs of the heavy or light chain variable regions are not modified.

In some embodiments, provided herein is a CD70 antibody or antigen binding portion thereof or other binding agent comprising a heavy chain variable region (VH) and a light chain variable region (VL), the VH and VL regions having amino acid sequences set forth in SEQ ID NO:9 and SEQ ID NO:10, respectively; wherein the heavy and light chain variable framework regions are optionally modified with from 1 to 8, 1 to 6, 1 to 4 or 1 to 2 conservative amino acid substitutions in the framework regions and wherein the CDRs of the heavy or light chain variable regions are not modified. In some embodiments, provided herein is a CD70 antibody or antigen binding portion thereof or other binding agent comprising a heavy chain variable region (VH) and a light chain variable region (VL), the VH and VL regions having amino acid sequences set forth in SEQ ID NO:9 and SEQ ID NO:10, respectively; wherein the heavy and light chain variable framework regions are optionally modified with from 1 to 8, 1 to 6, 1 to 4 or 1 to 2 amino acid substitutions, deletions or insertions in the framework regions and wherein the CDRs of the heavy or light chain variable regions are not modified.

In some embodiments, provided herein is a CD70 antibody or antigen binding portion thereof or other binding agent comprising a heavy chain variable region (VH) and a light chain variable region (VL), the VH and VL regions having amino acid sequences set forth in SEQ ID NO:11 and SEQ ID NO:12; respectively; wherein the heavy and light chain variable framework regions are optionally modified with from 1 to 8, 1 to 6, 1 to 4 or 1 to 2 conservative amino acid substitutions in the framework regions and wherein the CDRs of the heavy or light chain variable regions are not modified. In some embodiments, provided herein is a binding agent comprising a heavy chain variable region (VH) and a light chain variable region (VL), the VH and VL regions having amino acid sequences set forth in SEQ ID NO:11 and SEQ ID NO:12; respectively; wherein the heavy and light chain variable framework regions are optionally modified with from 1 to 8, 1 to 6, 1 to 4 or 1 to 2 amino acid substitutions, deletions or insertions in the framework regions and wherein the CDRs of the heavy or light chain variable regions are not modified.

In any of these embodiments, the functional activity of the CD70 binding antibody or antigen binding portion thereof or other binding agent includes specifically binding to CD70. Additional functional activities include depletion of CD70+ cells (e.g., cancer cells or autoimmune cells). Additionally, a CD70 antibody or antigen binding portion thereof or other binding agent having functional activity means the polypeptide exhibits activity similar to, or better than, the activity of a reference antibody or antigen-binding portion thereof as described herein (e.g., a reference CD70 binding antibody or antigen binding portion thereof comprising (i) a heavy chain variable region having the amino acid sequence set forth in SEQ ID NO:1 and (ii) a light chain variable region having the amino acid sequence set forth in SEQ ID NO:2 or a variant thereof, as described herein), as measured in a particular assay, such as, for example, a biological assay, with or without dose dependency. In the case where dose dependency does exist, it need not be identical to that of the reference antibody or antigen-binding portion thereof, but rather substantially similar to or better than the dose-dependence in a given activity as compared to the reference antibody or antigen-binding portion thereof as described herein (i.e., the candidate polypeptide will exhibit greater activity relative to the reference antibody).

For conservative substitutions, amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); and (4) basic: Lys (K), Arg (R), His (H).

Alternatively, for conservative substitutions naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; and (6) aromatic: Trp, Tyr, Phe. Non-conservative substitutions will entail exchanging a member of one of these classes or another class.

Particular conservative substitutions include, for example; Ala to Gly or to Ser; Arg to Lys; Asn to Gln or to His; Asp to Glu; Cys to Ser; Gln to Asn; Glu to Asp; Gly to Ala or to Pro; His to Asn or to Gln; Ile to Leu or to Val; Leu to lie or to Val; Lys to Arg, to Gln or to Glu; Met to Leu, to Tyr or to lie; Phe to Met, to Leu or to Tyr; Ser to Thr; Thr to Ser; Trp to Tyr; Tyr to Trp; and/or Phe to Val, to lie or to Leu.

In some embodiments, a conservatively modified variant of a CD70 antibody or antigen binding portion thereof preferably is at least 90%, at least 91%, at least 92%, at least 93%, at least 94%, at least 95%, at least 96%, at least 97%, at least 98%, at least 99%, or more, identical to the reference VH or VL sequence, wherein the VH and VL CDRs are not modified. The degree of homology (percent identity) between the reference and modified sequence can be determined, for example, by comparing the two sequences using freely available computer programs commonly employed for this purpose on the world wide web (e.g. BLASTp or BLASTn with default settings).

In some embodiments, the VH and VL amino acid sequences collectively have no more than 8 or 6 or 4 or 2 or 1 conservative amino acid substitutions in the framework regions, as compared to the amino acid sequences of the unmodified VH and VL regions. In some embodiments, the VH and VL amino acid sequences collectively have 8 to 1, or 6 to 1, or 4 to 1, or 2 to 1 conservative amino acid substitutions in the framework regions, as compared to the amino acid sequences of the unmodified VH and VL regions. In some embodiments, the VH and VL amino acid sequences collectively have no more than 8 or 6 or 4 or 2 or 1 amino acid substitutions, deletions or insertions in the framework regions, as compared to the amino acid sequences of the unmodified VH and VL regions. In some embodiments, the VH and VL amino acid sequences have 8 to 1, 6 to 1, 4 to 1, or 2 to 1 conservative amino acid substitutions in the framework regions, as compared to the amino acid sequences of the unmodified VH and VL regions. In some embodiments, the VH and VL amino acid sequences collectively have no more than 8 or 6 or 4 or 2 or 1 amino acid substitutions, deletions or insertions, as compared to the amino acid sequences of the unmodified VH and VL regions.

Modification of a native (or reference) amino acid sequence can be accomplished by any of a number of techniques known to one of skill in the art. Mutations can be introduced, for example, at particular loci by synthesizing oligonucleotides containing the desired mutant sequence, flanked by restriction sites enabling ligation to fragments of the native sequence. Following ligation, the resulting reconstructed sequence encodes a variant having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion desired. Techniques for making such alterations are very well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties.

### Constant Regions

In some embodiments, a CD70 antibody or antigen-binding portion thereof or other binding agent has fully human constant regions. In some embodiments, a CD70 antibody or antigen-binding portion thereof or other binding agent has humanized constant regions. In some embodiments, a CD70 antibody or antigen-binding portion thereof or other binding agent has non-human constant regions. An immunoglobulin constant region refers to a heavy or light chain constant region. Human heavy chain and light chain constant region amino acid sequences are known in the art. A constant region can be of any suitable type, which can be selected from the classes of immunoglobulins, IgA, IgD, IgE, IgG, and IgM. Several immunoglobulin classes can be further divided into isotypes, e.g., IgGI, IgG2, IgG3, IgG4, or IgAI, and IgA2. The heavy-chain constant regions (Fc) that correspond to the different classes of immunoglobulins can be a, 5, E, y, and p, respectively. The light chains can be one of either kappa (or K) and lambda (or A).

In some embodiments, a constant region can have an IgGI isotype. In some embodiments, a constant region can have an IgG2 isotype. In some embodiments, a constant region can have an IgG3 isotype. In some embodiments, a constant region can have an IgG4 isotype. In some embodiments, an Fc domain can have a hybrid isotype comprising constant regions from two or more isotypes. In some embodiments, an immunoglobulin constant region can be an IgG1 or IgG4 constant region. In some embodiments, a CD70 antibody heavy chain is of the IgG1 isotype and has the amino acid sequence set forth in SEQ ID NO:28. In some embodiments, a CD70 antibody light chain is of the kappa isotype and has the amino acid sequence set forth in SEQ ID NO:29.

Furthermore, a CD70 antibody or an antigen-binding portion thereof or other binding agent may be part of a larger binding agent formed by covalent or noncovalent association of the antibody or antigen binding portion with one or more other proteins or peptides. Relevant to such binding agents are the use, for example, of the streptavidin core region in order to prepare a tetrameric scFv molecule (Kipriyanov, S. M., et al. (1995), Human Antibodies and Hybridomas 6:93-101) and the use of a cysteine residue, a marker peptide and a C-terminal polyhistidinyl peptide, e.g. hexahistidinyl tag (‘hexahistidinyl tag’ disclosed as SEQ ID NO: 30) in order to produce bivalent and biotinylated scFv molecules (Kipriyanov, S. M., et al. (1994) Mol. Immunol. 31:10471058).

### Fc Domain Modifications to Alter Effector Function

In some embodiments, an Fc region or Fc domain of a CD70 antibody or antigen binding portion thereof or other binding agent has substantially no binding to at least one Fc receptor selected from FcyRI (CD64), FcyRIIA (CD32a), FcyRIIB (CD32b), FcyRIIIA (CD16a), and FcyRIIIB (CD16b). In some embodiments, an Fc region or domain exhibits substantially no binding to any of the Fc receptors selected from FcyRI (CD64), FcyRIIA (CD32a), FcyRIIB (CD32b), FcyRIIIA (CD16a), and FcyRIIIB (CD16b). As used herein, “substantially no binding” refers to weak to no binding to a selected Fcgamma receptor or receptors. In some embodiments, “substantially no binding” refers to a reduction in binding affinity (i.e., increase in Kd) to a Fc gamma receptor of at least 1000-fold. In some embodiments, an Fc domain or region is an Fc null. As used herein, an “Fc null” refers to an Fc region or Fc domain that exhibits weak to no binding to any of the Fcgamma receptors. In some embodiments, an Fc null domain or region exhibits a reduction in binding affinity (i.e., increase in Kd) to Fc gamma receptors of at least 1000-fold.

In some embodiments, an Fc domain has reduced or substantially no effector function activity. As used herein, “effector function activity” refers to antibody dependent cellular cytotoxicity (ADCC), antibody dependent cellular phagocytosis (ADCP) and/or complement dependent cytotoxicity (CDC). In some embodiments, an Fc domain exhibits reduced ADCC, ADCP or CDC activity, as compared to a wildtype Fc domain. In some embodiments, an Fc domain exhibits a reduction in ADCC, ADCP and CDC, as compared to a wildtype Fc domain. In some embodiments, an Fc domain exhibits substantially no effector function (i.e., the ability to stimulate or effect ADCC, ADCP or CDC). As used herein, “substantially no effector function” refers to a reduction in effector function activity of at least 1000-fold, as compared to a wildtype or reference Fc domain.

In some embodiments, an Fc domain has reduced or no ADCC activity. As used herein reduced or no ADCC activity refers to a decrease in ADCC activity of an Fc domain by a factor of at least 10, at least 20, at least 30, at least 50, at least 100 or at least 500.

In some embodiments, an Fc domain has reduced or no CDC activity. As used herein reduced or no CDC activity refers to a decrease in CDC activity of an Fc domain by of a factor of at least 10, at least 20, at least 30, at least 50, at least 100 or at least 500.

In vitro and/or in vivo cytotoxicity assays can be conducted to confirm the reduction/depletion of ADCC and/or CDC activity. For example, Fc receptor (FcR) binding assays can be conducted to ensure that the antibody lacks Fcgamma receptor binding (hence likely lacking ADCC activity). The primary cells for mediating ADCC, NK cells, express FcgammaRIII only, whereas monocytes express FcgammaRI, FcgammaRII and FcgammaRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest are described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); U.S. Pat. No. 5,821,337 (see Bruggemann, M. et al., J. Exp. Med. 166:1351-1361 (1987)). Alternatively, non-radioactive assay methods may be employed (see, for example, ACTI™ non-radioactive cytotoxicity assay for flow cytometry (CellTechnology, Inc. Mountain View, Calif.; and CytoTox 96™ non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al., Proc. Nat'l Acad. Sci. USA 95:652-656 (1998).

C1q binding assays may also be carried out to confirm that an antibody or Fc domain or region is unable to bind C1q and hence lacks CDC activity or has reduced CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)).

In some embodiments, an Fc domain has reduced or no ADCP activity. As used herein reduced or no ADCP activity refers to a decrease in ADCP activity of an Fc domain by a factor of at least 10, at least 20, at least 30, at least 50, at least 100 or at least 500.

ADCP binding assays may also be carried out to confirm that an antibody or Fc domain or region lacks ADCP activity or has reduced ADCP activity. See, e.g., US20190079077 and US20190048078 and the references disclosed therein.

A CD70 antibody or antigen binding portion thereof or other binding agent with reduced effector function activity includes those with substitution of one or more of Fc region residues, such as, for example, 238, 265, 269, 270, 297, 327 and 329, according to the EU number of Kabat (see, e.g., U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine, according to the EU numbering of Kabat (see U.S. Pat. No. 7,332,581). Certain antibody variants with diminished binding to FcRs are also known. (See, e.g., U.S. Pat. No. 6,737,056; WO 2004/056312, and Shields et al., J. Biol. Chem. 9(2): 6591-6604 (2001).) A CD70 antibody or antigen binding portion thereof or other binding agent with diminished binding to FcRs can be prepared containing such amino acid modifications.

In some embodiments, a CD70 antibody or antigen binding portion thereof or other binding agent comprises an Fc domain or region with one or more amino acid substitutions which diminish FcgammaR binding, e.g., substitutions at positions 234 and 235 of the Fc region (EU numbering of residues). In some embodiments, the substitutions are L234A and L235A (LALA), according to the EU numbering of Kabat. In some embodiments, the Fc domain comprises D265A and/or P329G in an Fc region derived from a human IgG1 Fc region, according to the EU numbering of Kabat. In some embodiments, the substitutions are L234A, L235A and P329G (LALA-PG), according to the EU numbering of Kabat, in an Fc region derived from a human IgG1 Fc region. (See, e.g., WO 2012/130831). In some embodiments, the substitutions are L234A, L235A and D265A (LALA-DA) in an Fc region derived from a human IgG1 Fc region, according to the EU numbering of Kabat.

In some embodiments, alterations are made in the Fc region that result in altered (i.e., either diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

### Methods of Making Antibodies, Antigen Binding Portions and Other Binding Agents

In various embodiments, CD70 antibodies, antigen binding portions thereof and other binding agents can be produced in human, murine or other animal-derived cells lines. Recombinant DNA expression can be used to produce CD70 antibodies, antigen binding portions thereof and other binding agents. This allows the production of CD70 antibodies as well as a spectrum of CD70 antigen binding portions and other binding agents (including fusion proteins) in a host species of choice. The production of CD70 antibodies, antigen binding portions thereof and other binding agents in bacteria, yeast, transgenic animals and chicken eggs are also alternatives for cell-based production systems. The main advantages of transgenic animals are potential high yields from renewable sources.

In some embodiments, a CD70 VH polypeptide having the amino acid sequence set forth in SEQ ID NOs:3, 5, 7, 9 or 11 is encoded by a nucleic acid. In some embodiments, a CD70 VL polypeptide having the amino acid sequence set forth in SEQ ID NOs: 4, 6, 8, 10, or 12 is encoded by a nucleic acid. In some embodiments, a nucleic acid encodes a CD70 VH polypeptide having the amino acid sequence set forth in SEQ ID NOs:3, 5, 7, 9 or 11. In some embodiments, a nucleic acid encodes a CD70 VL polypeptide having the amino acid sequence set forth in SEQ ID NOs: 4, 6, 8, 10, or 12. In some embodiments, the nucleic acid encodes a CD70 VH polypeptide having the amino acid sequence set forth in SEQ ID NO:3. In some embodiments, the nucleic acid encodes a CD70 VH polypeptide having the amino acid sequence set forth in SEQ ID NO:5. In some embodiments, the nucleic acid encodes a CD70 VH polypeptide having the amino acid sequence set forth in SEQ ID NO:7. In some embodiments, the nucleic acid encodes a CD70 VH polypeptide having the amino acid sequence set forth in SEQ ID NO:9. In some embodiments, the nucleic acid encodes a CD70 VH polypeptide having the amino acid sequence set forth in SEQ ID NO:11. In some embodiments, the nucleic acid encodes a CD70 VH polypeptide having the amino acid sequence set forth in SEQ ID NO:4. In some embodiments, the nucleic acid encodes a CD70 VH polypeptide having the amino acid sequence set forth in SEQ ID NO:6. In some embodiments, the nucleic acid encodes a CD70 VH polypeptide having the amino acid sequence set forth in SEQ ID NO:8. In some embodiments, the nucleic acid encodes a CD70 VH polypeptide having the amino acid sequence set forth in SEQ ID NO:10. In some embodiments, the nucleic acid encodes a CD70 VH polypeptide having the amino acid sequence set forth in SEQ ID NO:12.

In some embodiments, the nucleic acid encodes VH and VL polypeptides having the amino acid sequences set forth in SEQ ID NOs:3 and 4. In some embodiments, the nucleic acid encodes VH and VL polypeptides having the amino acid sequences set forth in SEQ ID NOs:5 and 6. In some embodiments, the nucleic acid encodes VH and VL polypeptides having the amino acid sequences set forth in SEQ ID NOs:7 and 8. In some embodiments, the nucleic acid encodes VH and VL polypeptides having the amino acid sequences set forth in SEQ ID NOs:9 and 10. In some embodiments, the nucleic acid encodes VH and VL polypeptides having the amino acid sequences set forth in SEQ ID NOs:11 and 12.

As used herein, the term “nucleic acid” or “nucleic acid sequence” or “polynucleotide sequence” or “nucleotide” refers to a polymeric molecule incorporating units of ribonucleic acid, deoxyribonucleic acid or an analog thereof. The nucleic acid can be either single-stranded or double-stranded. A single-stranded nucleic acid can be one strand nucleic acid of a denatured double-stranded DNA. In some embodiments, the nucleic acid can be a cDNA, e.g., a nucleic acid lacking introns.

Nucleic acid molecules encoding the amino acid sequence of a CD70 antibody, antigen binding portion thereof as well as other binding agents can be prepared by a variety of methods known in the art. These methods include, but are not limited to, preparation of synthetic nucleotide sequences encoding of a CD70 antibody, antigen binding portion or other binding agent(s). In addition, oligonucleotide-mediated (or site-directed) mutagenesis, PCR-mediated mutagenesis, and cassette mutagenesis can be used to prepare nucleotide sequences encoding a CD70 antibody or antigen binding portion as well as other binding agents. A nucleic acid sequence encoding at least a CD70 antibody, antigen binding portion thereof, binding agent, or a polypeptide thereof, as described herein, can be recombined with vector DNA in accordance with conventional techniques, such as, for example, blunt-ended or staggered-ended termini for ligation, restriction enzyme digestion to provide appropriate termini, filling in of cohesive ends as appropriate, alkaline phosphatase treatment to avoid undesirable joining, and ligation with appropriate ligases or other techniques known in the art. Techniques for such manipulations are disclosed, e.g., by Maniatis et al., Molecular Cloning, Lab. Manual (Cold Spring Harbor Lab. Press, NY, 1982 and 1989), and Ausubel et al., Current Protocols in Molecular Biology (John Wiley & Sons), 1987-1993, and can be used to construct nucleic acid sequences and vectors that encode a CD70 antibody or antigen binding portion thereof or a VH or VL polypeptide thereof or other binding agent.

A nucleic acid molecule, such as DNA, is said to be “capable of expressing” a polypeptide if it contains nucleotide sequences that contain transcriptional and translational regulatory information and such sequences are “operably linked” to nucleotide sequences that encode the polypeptide. An operable linkage is a linkage in which the regulatory DNA sequences and the DNA sequence sought to be expressed (e.g., a CD70 antibody or antigen binding portion thereof or other binding agent) are connected in such a way as to permit gene expression of a polypeptide(s) or antigen binding portions in recoverable amounts. The precise nature of the regulatory regions needed for gene expression may vary from organism to organism, as is well known in the analogous art. See, e.g., Sambrook et al., 1989; Ausubel et al., 1987-1993.

Accordingly, the expression of a CD70 antibody or antigen-binding portion thereof as described herein can occur in either prokaryotic or eukaryotic cells. Suitable hosts include bacterial or eukaryotic hosts, including yeast, insects, fungi, bird and mammalian cells either in vivo or in situ, or host cells of mammalian, insect, bird or yeast origin. The mammalian cell or tissue can be of human, primate, hamster, rabbit, rodent, cow, pig, sheep, horse, goat, dog or cat origin, but any other mammalian cell may be used. Further, by use of, for example, the yeast ubiquitin hydrolase system, in vivo synthesis of ubiquitin-transmembrane polypeptide fusion proteins can be accomplished. The fusion proteins so produced can be processed in vivo or purified and processed in vitro, allowing synthesis of a CD70 antibody or antigen binding portion thereof or other binding agent as described herein with a specified amino terminus sequence. Moreover, problems associated with retention of initiation codon-derived methionine residues in direct yeast (or bacterial) expression maybe avoided. (See, e.g., Sabin et al., 7 Bio/Technol. 705 (1989); Miller et al., 7 Bio/Technol. 698 (1989).) Any of a series of yeast gene expression systems incorporating promoter and termination elements from the actively expressed genes coding for glycolytic enzymes produced in large quantities when yeast are grown in medium rich in glucose can be utilized to obtain recombinant CD70 antibodies or antigen-binding portions thereof or other binding agents. Known glycolytic genes can also provide very efficient transcriptional control signals. For example, the promoter and terminator signals of the phosphoglycerate kinase gene can be utilized.

Production of CD70 antibodies or antigen-binding portions thereof or other binding agents in insects can be achieved, for example, by infecting an insect host with a baculovirus engineered to express a polypeptide by methods known to those of ordinary skill in the art. See Ausubel et al., 1987-1993.

In some embodiments, the introduced nucleic acid sequence(s) (encoding a CD70 antibody or antigen binding portion thereof or other binding agent or a polypeptide thereof) is incorporated into a plasmid or viral vector capable of autonomous replication in a recipient host cell. Any of a wide variety of vectors can be employed for this purpose and are known and available to those of ordinary skill in the art. See, e.g., Ausubel et al., 1987-1993. Factors of importance in selecting a particular plasmid or viral vector include: the ease with which recipient cells that contain the vector may be recognized and selected from those recipient cells which do not contain the vector; the number of copies of the vector which are desired in a particular host; and whether it is desirable to be able to “shuttle” the vector between host cells of different species.

Exemplary prokaryotic vectors known in the art include plasmids such as those capable of replication in E. coli. Other gene expression elements useful for the expression of DNA encoding CD70 antibodies or antigen-binding portions thereof or other binding agents include, but are not limited to (a) viral transcription promoters and their enhancer elements, such as the SV40 early promoter. (Okayama et al., 3 Mol. Cell. Biol. 280 (1983)), Rous sarcoma virus LTR (Gorman et al., 79 PNAS 6777 (1982)), and Moloney murine leukemia virus LTR (Grosschedl et al., 41 Cell 885 (1985)); (b) splice regions and polyadenylation sites such as those derived from the SV40 late region (Okayarea et al., 1983), and (c) polyadenylation sites such as in SV40 (Okayama et al., 1983). Immunoglobulin-encoding DNA genes can be expressed as described by Liu et al., infra, and Weidle et al., 51 Gene 21 (1987), using as expression elements the SV40 early promoter and its enhancer, the mouse immunoglobulin H chain promoter enhancers, SV40 late region mRNA splicing, rabbit S-globin intervening sequence, immunoglobulin and rabbit S-globin polyadenylation sites, and SV40 polyadenylation elements.

For immunoglobulin encoding nucleotide sequences, the transcriptional promoter can be, for example, human cytomegalovirus, the promoter enhancers can be cytomegalovirus and mouse/human immunoglobulin.

In some embodiments, for expression of DNA coding regions in rodent cells, the transcriptional promoter can be a viral LTR sequence, the transcriptional promoter enhancers can be either or both the mouse immunoglobulin heavy chain enhancer and the viral LTR enhancer, and the polyadenylation and transcription termination regions. In other embodiments, DNA sequences encoding other proteins are combined with the above-recited expression elements to achieve expression of the proteins in mammalian cells.

Each coding region or gene fusion is assembled in, or inserted into, an expression vector. Recipient cells capable of expressing the CD70 variable region(s) or antigen binding portions thereof or other binding agents are then transfected singly with nucleotides encoding a CD70 antibody or an antibody polypeptide or antigen-binding portion thereof or other binding agent, or are co-transfected with a polynucleotide(s) encoding VH and VL chain coding regions or other binding agents. The transfected recipient cells are cultured under conditions that permit expression of the incorporated coding regions and the expressed antibody chains or intact antibodies or antigen binding portions or other binding agents are recovered from the culture.

In some embodiments, the nucleic acids containing the coding regions encoding a CD70 antibody or antigen-binding portion thereof or other binding agent are assembled in separate expression vectors that are then used to co-transfect a recipient host cell. Each vector can contain one or more selectable genes. For example, in some embodiments, two selectable genes are used, a first selectable gene designed for selection in a bacterial system and a second selectable gene designed for selection in a eukaryotic system, wherein each vector has a set of coding regions. This strategy results in vectors which first direct the production, and permit amplification, of the nucleotide sequences in a bacterial system. The DNA vectors so produced and amplified in a bacterial host are subsequently used to co-transfect a eukaryotic cell, and allow selection of a co-transfected cell carrying the desired transfected nucleic acids (e.g., containing CD70 antibody heavy and light chains). Non-limiting examples of selectable genes for use in a bacterial system are the gene that confers resistance to ampicillin and the gene that confers resistance to chloramphenicol. Selectable genes for use in eukaryotic transfectants include the xanthine guanine phosphoribosyl transferase gene (designated gpt) and the phosphotransferase gene from Tn5 (designated neo). Alternatively the fused nucleotide sequences encoding VH and VL chains can be assembled on the same expression vector.

For transfection of the expression vectors and production of the CD70 antibodies or antigen binding portions thereof or other binding agents, the recipient cell line can be a Chinese Hamster ovary cell line (e.g., DG44) or a myeloma cell. Myeloma cells can synthesize, assemble and secrete immunoglobulins encoded by transfected immunoglobulin genes and possess the mechanism for glycosylation of the immunoglobulin. For example, in some embodiments, the recipient cell is the recombinant Ig-producing myeloma cell SP2/0. SP2/0 cells only produce immunoglobulins encoded by the transfected genes. Myeloma cells can be grown in culture or in the peritoneal cavity of a mouse, where secreted immunoglobulin can be obtained from ascites fluid.

An expression vector encoding a CD70 antibody or antigen-binding portion thereof or other binding agent can be introduced into an appropriate host cell by any of a variety of suitable means, including such biochemical means as transformation, transfection, protoplast fusion, calcium phosphate-precipitation, and application with polycations such as diethylaminoethyl (DEAE) dextran, and such mechanical means as electroporation, direct microinjection and microprojectile bombardment. Johnston et al., 240 Science 1538 (1988), as known to one of ordinary skill in the art.

Yeast provides certain advantages over bacteria for the production of immunoglobulin heavy and light chains. Yeasts carry out post-translational peptide modifications including glycosylation. A number of recombinant DNA strategies exist that utilize strong promoter sequences and high copy number plasmids which can be used for production of the desired proteins in yeast. Yeast recognizes leader sequences of cloned mammalian gene products and secretes polypeptides bearing leader sequences (i.e., pre-polypeptides). See, e.g., Hitzman et al., 11th Intl. Conf. Yeast, Genetics & Molec. Biol. (Montpelier, France, 1982).

Yeast gene expression systems can be routinely evaluated for the levels of production, secretion and the stability of antibodies, and assembled CD70 antibodies and antigen binding portions thereof and other binding agents. Various yeast gene expression systems incorporating promoter and termination elements from the actively expressed genes coding for glycolytic enzymes produced in large quantities when yeasts are grown in media rich in glucose can be utilized. Known glycolytic genes can also provide very efficient transcription control signals. For example, the promoter and terminator signals of the phosphoglycerate kinase (PGK) gene can be utilized. Another example is the translational elongation factor 1alpha promoter, such as that from Chinese hamster cells. A number of approaches can be taken for evaluating optimal expression plasmids for the expression of immunoglobulins in yeast. See II DNA Cloning 45, (Glover, ed., IRL Press, 1985) and e.g., U.S. Publication No. US 2006/0270045 A1.

Bacterial strains can also be utilized as hosts for the production of the antibody molecules or antigen binding portions thereof or other binding agents as described herein. E. coli K12 strains such as E. coli W3110, Bacillus species, enterobacteria such as Salmonella typhimurium or Serratia marcescens, and various Pseudomonas species can be used. Plasmid vectors containing replicon and control sequences that are derived from species compatible with a host cell are used in connection with these bacterial hosts. The vector carries a replication site, as well as specific genes which are capable of providing phenotypic selection in transformed cells. A number of approaches can be taken for evaluating the expression plasmids for the production of CD70 antibodies and antigen binding portions thereof and other binding agents in bacteria (see Glover, 1985; Ausubel, 1987, 1993; Sambrook, 1989; Colligan, 1992-1996).

Host mammalian cells can be grown in vitro or in vivo. Mammalian cells provide post-translational modifications to immunoglobulin molecules including leader peptide removal, folding and assembly of VH and VL chains, glycosylation of the antibody molecules, and secretion of functional antibody and/or antigen binding portions thereof or other binding agents.

Mammalian cells which can be useful as hosts for the production of antibody proteins, in addition to the cells of lymphoid origin described above, include cells of fibroblast origin, such as Vero or CHO-K1 cells. Exemplary eukaryotic cells that can be used to express immunoglobulin polypeptides include, but are not limited to, COS cells, including COS 7 cells; 293 cells, including 293-6E cells; CHO cells, including CHO—S and DG44 cells; PERC6™ cells (Crucell); and NSO cells. In some embodiments, a particular eukaryotic host cell is selected based on its ability to make desired post-translational modifications to the heavy chains and/or light chains. For example, in some embodiments, CHO cells produce polypeptides that have a higher level of sialylation than the same polypeptide produced in 293 cells.

In some embodiments, one or more CD70 antibodies or antigen-binding portions thereof or other binding agents can be produced in vivo in an animal that has been engineered or transfected with one or more nucleic acid molecules encoding the polypeptides, according to any suitable method.

In some embodiments, an antibody or antigen-binding portion thereof is produced in a cell-free system. Non-limiting exemplary cell-free systems are described, e.g., in Sitaraman et al., Methods Mol. Biol. 498: 229-44 (2009); Spirin, Trends Biotechnol. 22: 538-45 (2004); and Endo et al., Biotechnol. Adv. 21: 695-713 (2003).

Many vector systems are available for the expression of the VH and VL chains in mammalian cells (see Glover, 1985). Various approaches can be followed to obtain intact antibodies. As discussed above, it is possible to co-express VH and VL chains and optionally the associated constant regions in the same cells to achieve intracellular association and linkage of VH and VL chains into complete tetrameric H₂L₂ antibodies or antigen-binding portions thereof. The co-expression can occur by using either the same or different plasmids in the same host. Nucleic acids encoding the VH and VL chains or antigen binding portions thereof can be placed into the same plasmid, which is then transfected into cells, thereby selecting directly for cells that express both chains. Alternatively, cells can be transfected first with a plasmid encoding one chain, for example the VL chain, followed by transfection of the resulting cell line with a VH chain plasmid containing a second selectable marker. Cell lines producing antibodies, antigen-binding portions thereof via either route could be transfected with plasmids encoding additional copies of peptides, VH, VL, or VH plus VL chains in conjunction with additional selectable markers to generate cell lines with enhanced properties, such as higher production of assembled CD70 antibodies or antigen binding portions thereof or other binding agents or enhanced stability of the transfected cell lines.

Additionally, plants have emerged as a convenient, safe and economical alternative expression system for recombinant antibody production, which are based on large scale culture of microbes or animal cells. CD70 binding antibodies or antigen binding portions thereof or other binding agents can be expressed in plant cell culture, or plants grown conventionally. The expression in plants may be systemic, limited to sub-cellular plastids, or limited to seeds (endosperms). See, e.g., U.S. Patent Pub. No. 2003/0167531; U.S. Pat. Nos. 6,080,560; 6,512,162; and WO 0129242. Several plant-derived antibodies have reached advanced stages of development, including clinical trials (see, e.g., Biolex, N.C.).

For intact antibodies, the variable regions (VH and VL regions) of the CD70 antibodies are typically linked to at least a portion of an immunoglobulin constant region (Fc) or domain, typically that of a human immunoglobulin. Human constant region DNA sequences can be isolated in accordance with well-known procedures from a variety of human cells, such as immortalized B-cells (WO 87/02671). A CD70 binding antibody can contain both light chain and heavy chain constant regions. The heavy chain constant region can include CH1, hinge, CH2, CH3, and, optionally, CH4 regions. In some embodiments, the CH2 domain can be deleted or omitted.

Techniques described for the production of single chain antibodies (see, e.g. U.S. Pat. No. 4,946,778; Bird, Science 242:423-42 (1988); Huston et al., Proc. Natl. Acad. Sci. USA 85:5879-5883 (1988); and Ward et al., Nature 334:544-54 (1989); which are incorporated by reference herein in their entireties) can be adapted to produce single chain antibodies that specifically bind to CD70. Single chain antibodies are formed by linking the heavy and light chain variable regions of the Fv region via an amino acid bridge, resulting in a single chain polypeptide. Techniques for the assembly of functional Fv portions in E. coli can also be used (see, e.g. Skerra et al., Science 242:1038-1041 (1988); which is incorporated by reference herein in its entirety).

In some embodiments, an antigen binding portion or other binding agent comprises one or more scFvs. An scFv can be, for example, a fusion protein of the variable regions of the heavy (VH) and light chain (VL) variable regions of an antibody, connected with a short linker peptide of ten to about 25 amino acids. The linker is usually rich in glycine for flexibility, as well as serine or threonine for solubility, and can either connect the N-terminus of the VH with the C-terminus of the VL, or vice versa. This protein retains the specificity of the original antibody, despite removal of the constant regions and the introduction of the linker. scFv antibodies are, e.g. described in Houston, J. S., Methods in Enzymol. 203 (1991) 46-96. Methods for making scFv molecules and designing suitable peptide linkers are described in, for example, U.S. Pat. Nos. 4,704,692; 4,946,778; Raag and Whitlow, FASEB 9:73-80 (1995) and Bird and Walker, TIBTECH, 9: 132-137 (1991). scFv-Fcs have been described by Sokolowska-Wedzina et al., Mol. Cancer Res. 15(8):1040-1050, 2017.

In some embodiments, an antigen binding portion or other binding agent is a single-domain antibody is an antibody portion consisting of a single monomeric variable antibody domain. Single domains antibodies can be derived from the variable domain of the antibody heavy chain from camelids (e.g., nanobodies or VHH portions). Furthermore, a single-domain antibody can be an autonomous human heavy chain variable domain (aVH) or VNAR portions derived from sharks (see, e.g., Hasler et al., Mol. Immunol. 75:28-37, 2016).

Techniques for producing single domain antibodies (DABs or VHH) are known in the art, as disclosed for example in Cossins et al. (2006, Prot Express Purif 51:253-259) and Li et al. (Immunol. Lett. 188:89-95, 2017). Single domain antibodies may be obtained, for example, from camels, alpacas or llamas by standard immunization techniques. (See, e.g., Muyldermans et al., TIBS 26:230-235, 2001; Yau et al., J Immunol Methods 281:161-75, 2003; and Maass et al., J Immunol Methods 324:13-25, 2007.) A VHH may have potent antigen-binding capacity and can interact with epitopes that are inacessible to conventional VH-VL pairs (see, e.g., Muyldermans et al., 2001). Alpaca serum IgG contains about 50% camelid heavy chain only IgG antibodies (HCAbs) (see, e.g., Maass et al., 2007). Alpacas may be immunized with antigens and VHHs can be isolated that bind to and neutralize the target antigen (see, e.g., Maass et al., 2007). PCR primers that amplify alpaca VHH coding sequences have been identified and can be used to construct alpaca VHH phage display libraries, which can be used for antibody fragment isolation by standard biopanning techniques well known in the art (see, e.g., Maass et al., 2007).

Techniques for making multispecific antibodies include, but are not limited to, recombinant co-expression of two immunoglobulin heavy chain-light chain pairs having different specificities (see, e.g., Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168; Carter (2001), J Immunol Methods 248, 7-15). Multi-specific antibodies may also be made by engineering electrostatic steering effects for making antibody Fc-heterodimeric molecules (see, e.g., WO 2009/089004A1); cross-linking of two or more antibodies or antigen binding portions thereof (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody portions (see, e.g., Hollinger et al., Proc. Natl. Acad. Sci. USA, 90:6444-6448 (1993)); and using single-chain Fv (scFv) dimers (see, e.g. Gruber et al., J. Immunol., 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” also can be binding agents (see, e.g. US 2006/0025576A1).

The binding agents (e.g., antibodies or antigen binding portions) herein also include a “Dual Acting FAb” or “DAF” comprising an antigen binding site that binds to two different antigens (see, e.g., US 2008/0069820 and Bostrom et al., 2009, Science 323:1610-14). “Crossmab” antibodies are also included herein (see e.g. WO 2009/080251, WO 2009/080252, WO2009/080253, WO2009/080254, and WO2013/026833).

In some embodiments, the binding agents comprise different antigen-binding sites, fused to one or the other of the two subunits of the Fc domain; thus, the two subunits of the Fc domain may be comprised in two non-identical polypeptide chains. Recombinant co-expression of these polypeptides and subsequent dimerization leads to several possible combinations of the two polypeptides. To improve the yield and purity of the bispecific molecules in recombinant production, it will thus be advantageous to introduce in the Fc domain of the binding agent a modification promoting the association of the desired polypeptides.

Generally, this method involves replacement of one or more amino acid residues at the interface of the two Fc domains by charged amino acid residues so that homodimer formation becomes electrostatically unfavorable but heterodimerization electrostatically favorable.

In some embodiments, a binding agent is a “bispecific T cell engager” or BiTE (see, e.g., WO2004/106381, WO2005/061547, WO2007/042261, and WO2008/119567). This approach utilizes two antibody variable domains arranged on a single polypeptide. For example, a single polypeptide chain can include two single chain Fv (scFv) portions, each having a variable heavy chain (VH) and a variable light chain (VL) domain separated by a polypeptide linker of a length sufficient to allow intramolecular association between the two domains. This single polypeptide further includes a polypeptide spacer sequence between the two scFvs. Each scFv recognizes a different epitope, and these epitopes may be specific for different proteins, such that both proteins are bound by the BiTE.

As it is a single polypeptide, the bispecific T cell engager may be expressed using any prokaryotic or eukaryotic cell expression system known in the art, e.g., a CHO cell line. However, specific purification techniques (see, e.g., EP1691833) may be necessary to separate monomeric bispecific T cell engagers from other multimeric species, which may have biological activities other than the intended activity of the monomer. In one exemplary purification scheme, a solution containing secreted polypeptides is first subjected to a metal affinity chromatography, and polypeptides are eluted with a gradient of imidazole concentrations. This eluate is further purified using anion exchange chromatography, and polypeptides are eluted using with a gradient of sodium chloride concentrations. Finally, this eluate is subjected to size exclusion chromatography to separate monomers from multimeric species. In some embodiments, a binding agent that is a bispecific antibody is composed of a single polypeptide chain comprising two single chain FV portions (scFV) fused to each other by a peptide linker.

In some embodiments, a binding agent is multispecific, such as an IgG-scFV. IgG-scFv formats include IgG(H)-scFv, scFv-(H)IgG, IgG(L)-scFv, svFc-(L)IgG, 2scFV-IgG and IgG-2scFv. These and other bispecific antibody formats and methods of making them have been described in for example, Brinkmann and Kontermann, MAbs 9(2):182-212 (2017); Wang et al., Antibodies, 2019, 8, 43; Dong et al., 2011, MAbs 3:273-88; Natsume et al., J. Biochem. 140(3):359-368, 2006; Cheal et al., Mol. Cancer Ther. 13(7):1803-1812, 2014; and Bates and Power, Antibodies, 2019, 8, 28.

Igg-like dual-variable domain antibodies (DVD-Ig) have been described by Wu et al., 2007, Nat Biotechnol 25:1290-97; Hasler et al., Mol. Immunol. 75:28-37, 2016 and in WO 08/024188 and WO 07/024715. Triomabs have been described by Chelius et al., MAbs 2(3):309-319, 2010. 2-in-1-IgGs have been described by Kontermann et al., Drug Discovery Today 20(7):838-847, 2015. Tanden antibody or TandAb have been described by Kontermann et al., id. ScFv-HSA-scFv antibodies have also been described by Kontermann et al. (id.).

Intact (e.g., whole) antibodies, their dimers, individual light and heavy chains, or antigen binding portions thereof and other binding agents can be recovered and purified by known techniques, e.g., immunoadsorption or immunoaffinity chromatography, chromatographic methods such as HPLC (high performance liquid chromatography), ammonium sulfate precipitation, gel electrophoresis, or any combination of these. See generally, Scopes, Protein Purification (Springer-Verlag, N.Y., 1982). Substantially pure CD70 binding antibodies or antigen binding portions thereof or other binding agents of at least about 90% to 95% homogeneity are advantageous, as are those with 98% to 99% or more homogeneity, particularly for pharmaceutical uses. Once purified, partially or to homogeneity as desired, an intact CD70 antibody or antigen binding portions thereof or other binding agent can then be used therapeutically or in developing and performing assay procedures, immunofluorescent staining, and the like. See generally, Vols. I & II Immunol. Meth. (Lefkovits & Pernis, eds., Acad. Press, NY, 1979 and 1981).

### Antibody Drug Conjugates

In some embodiments, a CD70 antibody, antigen binding portion or other binding agent as described herein is part of a CD70 antibody drug conjugate (also referred to as a CD70 conjugate or CD70 ADC). In some embodiments, the CD70 antibody, antigen binding portion or other binding agent is attached to at least one linker, and at least one drug is attached to each linker. As used herein, in the context of a conjugate, the term “drug” refers to cytotoxic agents (such as chemotherapeutic agents or drugs), immunomodulatory agents, nucleic acids (including siRNAs), growth inhibitory agents, toxins (e.g., protein toxins, enzymatically active toxins of bacterial, fungal, plant, or animal origin, or fragments thereof), radioactive isotopes, PROTACs and other compounds that are active against target cells when delivered to those cells.

### Cytotoxic Agents

In some embodiments, a CD70 conjugate includes at least one drug that is cytotoxic agent. A “cytotoxic agent” refers to an agent that has a cytotoxic effect on a cell. A “cytotoxic effect” refers to the depletion, elimination and/or the killing of a target cell(s). Cytotoxic agents include, for example, tubulin disrupting agents, topoisomerase inhibitors, DNA minor groove binders, and DNA alkylating agents.

Tubulin disrupting agents include, for example, auristatins, dolastatins, tubulysins, colchicines, vinca alkaloids, taxanes, cryptophycins, maytansinoids, hemiasterlins, as well as other tubulin disrupting agents. Auristatins are derivatives of the natural product dolastatin 10. Exemplary auristatins include MMAE (N-methylvaline-valine-dolaisoleuine-dolaproine-norephedrine), MMAF (N-methylvaline-valine-dolaisoleuine-dolaproine-phenylalanine) and AFP (see WO2004/010957 and WO2007/008603). Other auristatin like compounds are disclosed in, for example, Published US Application Nos. US2021/0008099, US2017/0121282, US2013/0309192 and US2013/0157960. Dolastatins include, for example, dolastatin 10 and dolastatin 15 (see, e.g., Pettit et al., J. Am. Chem. Soc., 1987, 109, 6883-6885; Pettit et al., Anti-Cancer Drug Des., 1998, 13, 243-277; and Published US Application US2001/0018422). Additional dolastatin derivatives contemplated for use herein are disclosed in U.S. Pat. No. 9,345,785, incorporated herein by reference.

Tubulysins include, but are not limited to, tubulysin D, tubulysin M, tubuphenylalanine and tubutyrosine. WO2017/096311 and WO/2016-040684 describe tubulysin analogs including tubulysin M.

Colchicines include, but are not limited to, colchicine and CA-4.

Vinca alkaloids include, but are not limited to, vinblastine (VBL), vinorelbine (VRL), vincristine (VCR) and vindesine (VOS).

Taxanes include, but are not limited to, paclitaxel and docetaxel.

Cryptophycins include but are not limited to cryptophycin-1 and cryptophycin-52. Maytansinoids include, but are not limited to, maytansine, maytansinol, maytansine analogs in DM1, DM3 and DM4, and ansamatocin-2. Exemplary maytansinoid drug moieties include those having a modified aromatic ring, such as: C-19-dechloro (U.S. Pat. No. 4,256,746) (prepared by lithium aluminum hydride reduction of ansamitocin P2); C-20-hydroxy (or C-20-demethyl)+/−C-19-dechloro (U.S. Pat. Nos. 4,361,650 and 4,307,016) (prepared by demethylation using Streptomyces or Actinomyces or dechlorination using LAH); and C-20-demethoxy, C-20-acyloxy (—OCOR), +/−dechloro (U.S. Pat. No. 4,294,757) (prepared by acylation using acyl chlorides), and those having modifications at other positions.

Maytansinoid drug moieties also include those having modifications such as: C-9-SH (U.S. Pat. No. 4,424,219) (prepared by the reaction of maytansinol with H₂S or P₂S₅); C-14-alkoxymethyl(demethoxy/CH₂OR) (U.S. Pat. No. 4,331,598); C-14-hydroxymethyl or acyloxymethyl (CH₂OH or CH₂OAc) (U.S. Pat. No. 4,450,254) (prepared from Nocardia); C-15-hydroxy/acyloxy (U.S. Pat. No. 4,364,866) (prepared by the conversion of maytansinol by Streptomyces); C-15-methoxy (U.S. Pat. Nos. 4,313,946 and 4,315,929) (isolated from Trewia nudiflora); C-18-N-demethyl (U.S. Pat. Nos. 4,362,663 and 4,322,348) (prepared by the demethylation of maytansinol by Streptomyces); and 4,5-deoxy (U.S. Pat. No. 4,371,533) (prepared by the titanium trichloride/LAH reduction of maytansinol).

Hemiasterlins include but are not limited to, hemiasterlin and HTI-286.

Other tubulin disrupting agents include taccalonolide A, taccalonolide B, taccalonolide AF, taccalonolide AJ, taccalonolide AI-epoxide, discodermolide, epothilone A, epothilone B, and laulimalide.

In some embodiments, a cytotoxic agent can be a topoisomerase inhibitor, such as a camptothecin. Exemplary camptothecins include, for example, camptothecin, irinotecan (also referred to as CPT-11), belotecan, (7-(2-(N-isopropylamino)ethyl)camptothecin), topotecan, 10-hydroxy-CPT, SN-38, exatecan and the exatecan analog DXd (see US20150297748). Other camptothecins are disclosed in WO1996/021666, WO00/08033, US2016/0229862 and WO2020/156189.

In some embodiments, a cytotoxic agent is a duocarmcycin, including the synthetic analogues, KW-2189 and CBI-TMI.

### Immune Modulatory Agents

In some embodiments, a drug is an immune modulatory agent. An immune modulatory agent can be, for example, a TLR7 and/or TLR8 agonist, a STING agonist, a RIG-I agonist or other immune modulatory agent.

In some embodiments, a drug is an immune modulatory agent, such as a TLR7 and/or TLR8 agonist. In some embodiments, a TLR7 agonist is selected from an imidazoquinoline, an imidazoquinoline amine, a thiazoquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine, heteroarothiadiazide-2,2-dioxide, a benzonaphthyridine, a guanosine analog, an adenosine analog, a thymidine homopolymer, ssRNA, CpG-A, PolyG10, and PolyG3. In some embodiments, the TLR7 agonist is selected from an imidazoquinoline, an imidazoquinoline amine, a thiazoquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine, heteroarothiadiazide-2,2-dioxide or a benzonaphthyridine. In some embodiments, a TLR7 agonist is a non-naturally occurring compound. Examples of TLR7 modulators include GS-9620, GSK-2245035, imiquimod, resiquimod, DSR-6434, DSP-3025, IMO-4200, MCT-465, MEDI-9197, 3M-051, SB-9922, 3M-052, Limtop, TMX-30X, TMX-202, RG-7863, RG-7795, and the compounds disclosed in US20160168164 (Janssen), US 20150299194 (Roche), US20110098248 (Gilead Sciences), US20100143301 (Gilead Sciences), and US20090047249 (Gilead Sciences).

In some embodiments, a TLR8 agonist is selected from a benzazepine, an imidazoquinoline, a thiazoloquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine or a ssRNA. In some embodiments, a TLR8 agonist is selected from a benzazepine, an imidazoquinoline, a thiazoloquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, and a tetrahydropyridopyrimidine. In some embodiments, a TLR8 agonist is a non-naturally occurring compound. Examples of TLR8 agonists include motolimod, resiquimod, 3M-051, 3M-052, MCT-465, IMO-4200, VTX-763, VTX-1463.

In some embodiments, a TLR8 agonist can be any of the compounds described WO2018/170179, WO2020/056198 and WO2020056194.

Other TLR7 and TLR8 agonists are disclosed in, for example, WO2016142250, WO2017046112, WO2007024612, WO2011022508, WO2011022509, WO2012045090, WO2012097173, WO2012097177, WO2017079283, US20160008374, US20160194350, US20160289229, U.S. Pat. No. 6,043,238, US20180086755 (Gilead), WO2017216054 (Roche), WO2017190669 (Shanghai De Novo Pharmatech), WO2017202704 (Roche), WO2017202703 (Roche), WO20170071944 (Gilead), US20140045849 (Janssen), US20140073642 (Janssen), WO2014056953 (Janssen), WO2014076221 (Janssen), WO2014128189 (Janssen), US20140350031 (Janssen), WO2014023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics), WO2018198091 (Novartis AG), and US20170131421 (Novartis AG).

In some embodiments, an immune modulatory agent is a STING agonist. Examples of STING agonists include, for example, those disclosed in WO2020059895, WO2015077354, WO2020227159, WO2020075790, WO2018200812, and WO2020074004.

In some embodiments, an immune modulatory agent is a RIG-1 agonist. Examples of RIG-1 agonists include KIN1148, SB-9200, KIN700, KIN600, KIN500, KIN100, KIN101, KIN400 and KIN2000.

### Toxins

In some embodiments, a drug is an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

### Radioisotopes

In some embodiments, a drug is a radioactive atom. A variety of radioactive isotopes are available for the production of radioconjugates. Examples include 1131, 1125, Y90, Re186, Re188, Sm153, Bi213, P32, Pb212 and radioactive isotopes of Lutetium (e.g., Lu177).

### PROTACs

In some embodiments, a drug is a proteolysis targeted chimera (PROTAC). PROTACs are described in, for example, Published US Application Nos. 20210015942, 20210015929, 20200392131, 20200216507, US20200199247 and US20190175612; the disclosures of which are incorporated by reference herein.

### Linkers

The CD70 conjugates typically comprise at least one linker, each linker having at least one drug attached to it. Typically, a conjugate includes a linker between a CD70 antibody (or antigen binding portion thereof or other binding agent) and the drug. In various embodiments, a linker may be a protease cleavable linker, an acid-cleavable linker, a disulfide linker, a disulfide-containing linker, or a disulfide-containing linker having a dimethyl group adjacent the disulfide bond (e.g., an SPDB linker) (see, e.g., Jain et al., Pharm. Res. 32:3526-3540 (2015); Chari et al., Cancer Res. 52:127-131 (1992); U.S. Pat. No. 5,208,020), a self-stabilizing linker (see, e.g., WO2018/031690 and WO2015/095755 and Jain et al., Pharm. Res. 32:3526-3540 (2015)), a non-cleavable linker (see, e.g., WO2007/008603), a photolabile linker, and/or a hydrophilic linker (see, e.g., WO2015/123679).

In some embodiments, a linker is a cleavable linker that is cleavable under intracellular conditions, such that cleavage of the linker releases the drug from the antibody (or antigen binding portion thereof or other binding agent) and/or linker in the intracellular environment. For example, in some embodiments, a linker is cleavable by a cleaving agent that is present in the intracellular environment (e.g., within a lysosome or endosome or caveolae). A linker can be, for example, a peptidyl linker that is cleaved by an intracellular peptidase or protease enzyme, including, but not limited to, a lysosomal or endosomal protease (see, e.g., WO2004/010957, US20150297748, US2008/0166363, US20120328564 and US20200347075). Typically, a peptidyl linker is at least one amino acid long or at least two amino acids long. Intracellular cleaving agents can include cathepsins B and D and plasmin, all of which are known to hydrolyze dipeptide drug derivatives resulting in the release of active drug inside target cells (see, e.g., Dubowchik and Walker, 1999, Pharm. Therapeutics 83:67-123). Most typical are peptidyl linkers that are cleavable by enzymes that are present in target antigen-expressing cells. For example, a peptidyl linker that is cleavable by the thiol-dependent protease cathepsin-B, which is highly expressed in cancerous tissue, can be used (e.g., a Phe-Leu or a Gly-Phe-Leu-Gly linker). Other such linkers are described, for example, in U.S. Pat. No. 6,214,345. In specific embodiments, the peptidyl linker cleavable by an intracellular protease is a Val-Cit linker or a Phe-Lys linker (see, e.g., U.S. Pat. No. 6,214,345, which describes the synthesis of doxorubicin with the val-cit linker) or Gly-Gly-Phe-Gly (SEQ ID NO: 35) linker (see, e.g., US2015/0297748). One advantage of using intracellular proteolytic release of the drug is that the drug is typically attenuated when conjugated and the serum stabilities of the conjugates are typically high. See also U.S. Pat. No. 9,345,785.

As used herein, the terms “intracellularly cleaved” and “intracellular cleavage” refer to a metabolic process or reaction inside a cell on an antibody drug conjugate, whereby the covalent attachment, e.g., the linker, between a drug (e.g., a cytotoxic agent) and the antibody is broken, resulting in the free drug, or other metabolite of the conjugate dissociated from the antibody inside the cell. The cleaved moieties of the conjugate are thus intracellular metabolites.

In some embodiments, a cleavable linker is pH-sensitive, i.e., sensitive to hydrolysis at certain pH values. Typically, a pH-sensitive linker is hydrolyzable under acidic conditions. For example, an acid-labile linker that is hydrolyzable in the lysosome (e.g., a hydrazone, semicarbazone, thiosemicarbazone, cis-aconitic amide, orthoester, acetal, ketal, or the like) can be used. (See, e.g., U.S. Pat. Nos. 5,122,368; 5,824,805; and 5,622,929; Dubowchik and Walker, 1999, Pharm. Therapeutics 83:67-123; Neville et al., 1989, Biol. Chem. 264:14653-14661.) Such linkers are relatively stable under neutral pH conditions, such as those in the blood, but are unstable at below pH 5.5 or 5.0, the approximate pH of the lysosome. In certain embodiments, a hydrolyzable linker is a thioether linker (such as, for example, a thioether attached to the drug via an acylhydrazone bond (see, e.g., U.S. Pat. No. 5,622,929)).

In some embodiments, a linker is cleavable under reducing conditions (e.g., a disulfide linker). A variety of disulfide linkers are known, including, for example, those that can be formed using SATA (N-succinimidyl-5-acetylthioacetate), SPDP (N-succinimidyl-3-(2-pyridyldithio)propionate), SPDB (N-succinimidyl-3-(2-pyridyldithio)butyrate) and SMPT (N-succinimidyl-oxycarbonyl-alpha-methyl-alpha-(2-pyridyl-dithio)toluene)-, SPDB and SMPT (see, e.g., Thorpe et al., 1987, Cancer Res. 47:5924-5931; Wawrzynczak et al., In Immunoconjugates: Antibody Conjugates in Radioimagery and Therapy of Cancer (C. W. Vogel ed., Oxford U. Press, 1987. See also U.S. Pat. No. 4,880,935.)

In some embodiments, the linker is a malonate linker (Johnson et al., 1995, Anticancer Res. 15:1387-93), a maleimidobenzoyl linker (Lau et al., 1995, Bioorg-Med-Chem. 3(10):1299-1304), or a 3′-N-amide analog (Lau et al., 1995, Bioorg-Med-Chem. 3(10):1305-12). In some embodiments, the linker unit is not cleavable, such as a maleimidocaproyl linker, and the drug is released by antibody degradation. (See U.S. Publication No. 2005/0238649).

In some embodiments, a linker is not substantially sensitive to the extracellular environment. As used herein, “not substantially sensitive to the extracellular environment,” in the context of a linker, means that no more than about 20%, typically no more than about 15%, more typically no more than about 10%, and even more typically no more than about 5%, no more than about 3%, or no more than about 1% of the linkers, in a sample of the antibody drug conjugate (ADC), are cleaved when the ADC is present in an extracellular environment (e.g., in plasma). Whether a linker is not substantially sensitive to the extracellular environment can be determined, for example, by incubating independently with plasma both (a) the ADC (the “ADC sample”) and (b) an equal molar amount of unconjugated antibody or drug (the “control sample”) for a predetermined time period (e.g., 2, 4, 8, 16, or 24 hours) and then comparing the amount of unconjugated antibody or drug present in the ADC sample with that present in control sample, as measured, for example, by high performance liquid chromatography.

In some embodiments, a linker promotes cellular internalization. In some embodiments, a linker promotes cellular internalization when conjugated to the drug such as a cytotoxic agent (i.e., in the milieu of the linker-drug moiety of the ADC as described herein). In yet other embodiments, a linker promotes cellular internalization when conjugated to both the drug and the CD70 antibody (i.e., in the milieu of the ADC as described herein).

A variety of linkers that can be used with the present compositions and methods are described in WO 2004010957. In some embodiments, a protease cleavable linker comprises a thiol-reactive spacer and a dipeptide. In some embodiments, the protease cleavable linker consists of a thiol-reactive maleimidocaproyl spacer, a valine-citrulline dipeptide, and a p-amino-benzyloxycarbonyl spacer.

In some embodiments, an acid cleavable linker is a hydrazine linker or a quaternary ammonium linker (see WO2017/096311 and WO2016/040684.)

In some embodiments, a linker is a self-stabilizing linker comprising a maleimide group as described in U.S. Pat. No. 9,504,756.

In some embodiments, a linker is a hydrophilic linker, such as, for example, the hydrophilic peptides in WO2015/123679 and the sugar alcohol polymer-based linkers disclosed in WO2013/012961 and WO2019/213046.

In other embodiments, conjugates of a CD70 antibody (or antigen binding portion or other binding agent) and a drug may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). Chelating agents for conjugation of a radionucleotide(s) to an antibody, antigen binding portion thereof or other binding agent have been described in, for example WO94/11026.

The conjugates of a CD70 antibodies (or antigen binding portion or other binding agent) include, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, IL., U.S.A).

In some embodiments, a linker is attached to a terminus of an amino acid sequence of an antibody, antigen binding portion or other binding agent or can be attached to a side chain modification of an antibody, antigen binding portion or other binding agent, such as the side chain of a lysine, serine, threonine, cysteine, tyrosine, aspartic acid, a non-natural amino acid residue, glutamine, or glutamic acid residue. An attachment between an antibody, antigen binding portion or other binding agent and a linker or drug can be via any of a number of bonds, for example but not limited to, an amide bond, an ester bond, an ether bond, a carbon-nitrogen bond, a carbon-carbon single double or triple bond, a disulfide bond, or a thioether bond. Functional groups that can form such bonds include, for example, amino groups, carboxyl groups, aldehyde groups, azide groups, alkyne and alkene groups, ketones, carbonates, carbonyl functionalities bonded to leaving groups such as cyano and succinimidyl and hydroxyl groups.

In some embodiments, a linker is attached to an antibody, antigen binding portion or other binding agent at an interchain disulfide. In some embodiments, a linker is connected to an antibody, antigen binding portion or other binding agent at a hinge cysteine residue. In some embodiments, a linker is attached to an antibody, antigen binding portion or other binding agent at an engineered cysteine residue. In some embodiments, a linker is connected to an antibody, antigen binding portion or other binding agent at a lysine residue. In some embodiments, a linker is connected to an antibody, antigen binding portion or other binding agent at an engineered glutamine residue. In some embodiments, a linker is connected to an antibody, antigen binding portion or other binding agent at an unnatural amino acid engineered into the heavy chain.

In some embodiments, a linker is attached to an antibody, antigen binding portion or other binding agent via a sulfhydryl group. In some embodiments, a linker is attached to an antibody, antigen binding portion or other binding agent via a primary amine. In some embodiments, a linker is attached via a link created between an unnatural amino acid on an antibody, antigen binding portion or other binding agent by reacting with oxime bond that was formed by modifying a ketone group with an alkoxyamine on a drug.

In some embodiments, a linker is attached to an antibody, antigen binding portion or other binding agent via Sortase A linker. A Sortase A linker can be created by a Sortase A enzyme fusing an LPXTG recognition motif (SEQ ID NO: 33) to an N-terminal GGG motif to regenerate a native amide bond.

### Exemplary Linker Drug Combinations

In some embodiments, a drug such as a tubulin disrupting agent, for example, an auristatin, is attached to a linker by a C-terminal carboxyl group that forms an amide bond with a linker (e.g., a Linker Unit (LU)) as described in U.S. Pat. No. 9,463,252, incorporated herein by reference). In some embodiments, a linker comprises at least one amino acid.

In some embodiments, a linker also comprises a stretcher unit and/or an amino acid unit. Exemplary stretcher units and amino acid units are described in U.S. Pat. Nos. 9,345,785 and 9,078,931, each of which is herein incorporated by reference.

In some embodiments, an antibody drug conjugate comprises an anti-CD70 antibody covalently linked to MMAE through an mc-val-cit-PAB linker.

In some embodiments, the CD70 conjugates have the following formula:

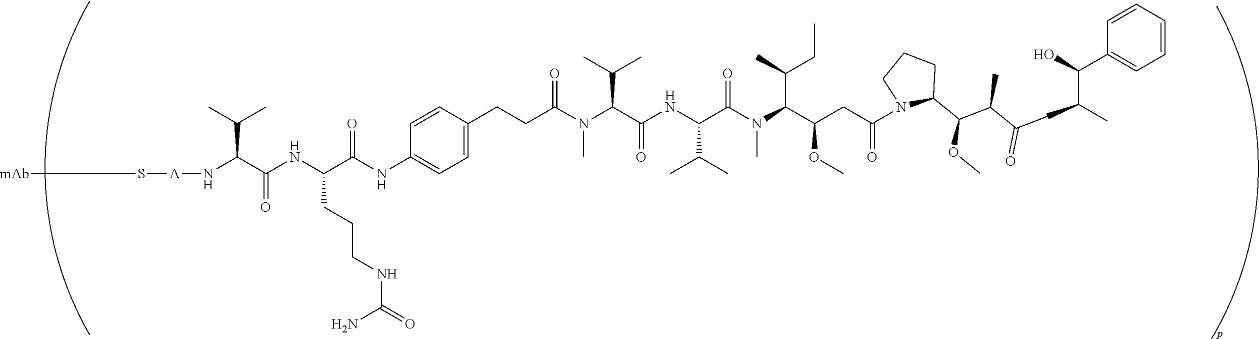

or a pharmaceutically acceptable salt thereof; wherein: mAb is a CD70 antibody, antigen binding portion thereof or other binding agent, S is a sulfur atom of the antibody, antigen binding portion or other binding agent, A is a Stretcher unit, and p is from about 3 to about 5, or from about 3 to about 8.

The drug loading is represented by p, the average number of drug molecules (e.g., cytotoxic agents) per antibody (or antigen binding portion or other binding agent) in a conjugate. For example, if p is about 4, the average drug loading taking into account all of the antibody (or antigen binding portion or other binding agent) present in the composition is about 4. In some embodiments, p ranges from about 3 to about 5, from about 3.6 to about 4.4, or from about 3.8 to about 4.2. In some embodiments, p can be about 3, about 4, or about 5. In some embodiments, p ranges from about 6 to about 8, more preferably from about 7.5 to about 8.4. In some embodiments, p can be about 6, about 7, or about 8.

The average number of drugs per antibody (or antigen binding portion or other binding agent) in a preparation may be characterized by conventional means such as mass spectroscopy, ELISA assay, and HPLC. The quantitative distribution of antibody-drug conjugates in terms of p may also be determined. In some instances, separation, purification, and characterization of homogeneous antibody-drug-conjugates where p is a certain value from antibody-drug-conjugates with other drug loadings may be achieved by means such as reverse phase HPLC or electrophoresis.

In some embodiments, a stretcher unit is capable of linking an antibody (or antigen binding portion or other binding agent) to an amino acid or peptide (e.g., a valine-citrulline peptide) via a sulfhydryl group of the antibody (or antigen binding portion or other binding agent). Sulfhydryl groups can be generated, for example, by reduction of the interchain disulfide bonds of a CD70 antibody (or antigen binding portion or other binding agent). For example, a stretcher unit can be linked to the antibody (or antigen binding portion or other binding agent) via the sulfur atoms generated from reduction of the interchain disulfide bonds of an antibody (or antigen binding portion or other binding agent). In some embodiments, stretcher units are linked to the antibody (or antigen binding portion or other binding agent) solely via the sulfur atoms generated from reduction of the interchain disulfide bonds of the antibody. In some embodiments, sulfhydryl groups can be generated by reaction of an amino group of a lysine moiety of a CD70 antibody (or antigen binding portion or other binding agent) with 2-iminothiolane (Traut's reagent) or other sulfhydryl generating reagents. In some embodiments, a CD70 antibody (or antigen binding portion or other binding agent) is a recombinant antibody and is engineered to carry one or more lysines. In some embodiments, a recombinant CD70 antibody (or antigen binding portion or other binding agent) is engineered to carry additional sulfhydryl groups, e.g., additional cysteines, such as engineered cysteines.

The synthesis and structure of MMAE is described in U.S. Pat. No. 6,884,869 incorporated by reference herein in its entirety and for all purposes. The synthesis and structure of exemplary stretcher units and methods for making antibody drug conjugates are described in, for example, U.S. Publication Nos. 2006/0074008 and 2009/0010945, each of which is incorporated herein by reference in its entirety.

Representative stretcher units are described within the square brackets of Formulas IIIa and IIIb of U.S. Pat. No. 9,211,319, and incorporated herein by reference.

In some embodiments, a CD70 conjugate comprises monomethyl auristatin E (MMAE) and a protease-cleavable linker. It is contemplated that the protease cleavable linker comprises a thiol-reactive spacer and a dipeptide. In various embodiments, the protease cleavable linker includes a thiol-reactive maleimidocaproyl spacer, a valine-citrulline (val-cit) dipeptide, and a p-amino-benzyloxycarbonyl or PAB spacer.

The abbreviation “PAB” refers to the self-immolative spacer:

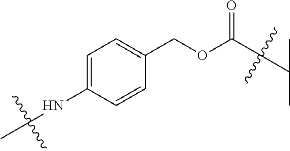

The abbreviation “MC” refers to the stretcher maleimidocaproyl:

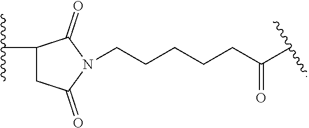

In other exemplary embodiments, a conjugate has the following general formula:

Ab-[L3]-[L2]-[L1]ₘ-AAₙ-drug

where Ab is a CD70 antibody (or antigen binding portion or other binding agent); the drug is, for example, a cytotoxic agent such as a tubulin-disrupting agent or topoisomerase inhibitor; L3 is a component of a linker comprising an antibody-coupling moiety (such as a stretcher unit) and one or more of acetylene (or azide) groups; L2 comprises an optional PEG (polyethylene glycol) azide (or acetylene) at one end, complementary to the acetylene (or azide) moiety in L3, and a reactive group such as carboxylic acid or hydroxyl group at the other end; L1 comprises a collapsible unit (e.g., a self-immolative group(s)), or a peptidase-cleavable moiety optionally attached to a collapsible unit, or an acid-cleavable moiety; AA is an amino acid; m is an integer with values of 0 or 1, and n is an integer with values of 0, 1, 2, 3, or 4. Such linkers can be assembled via click chemistry. (See, e.g., U.S. Pat. Nos. 7,591,944 and 7,999,083.)

In some embodiments, the drug is a camptothecin or a camptothecin (CPT) analog, such as irinotecan (also referred to as CPT-11), belotecan, topotecan, 10-hydroxy-CPT, exatecan, DXd or SN-38. Representative structures are shown below.

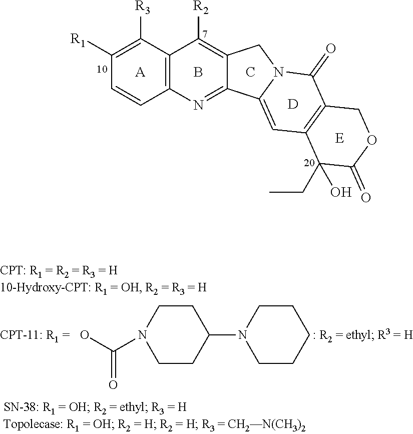

Referring to the conjugate formula Ab-[L3]-[L2]-[L1]ₘ-AAₙ-drug, in some embodiments, m is 0. Referring to the conjugate formula Ab-[L3]-[L2]-[L1]ₘ-AAₙ-drug, in some embodiments, L2 is absent. In such embodiments, an ester moiety is first formed between the carboxylic acid of an amino acid (AA) such as glycine, alanine, or sarcosine, or of a peptide such as glycylglycine, and a hydroxyl group of a drug, such as cytotoxic agent. In this example, the N-terminus of the amino acid or polypeptide may be protected as a Boc or a Fmoc or a monomethoxytrityl (MMT) derivative, which is deprotected after formation of an ester bond with the hydroxyl group of the cytotoxic agent. Selective removal of an amine-protecting group, in the presence of a BOC protecting group at a hydroxyl position of the cytotoxic agent containing an additional hydroxyl group(s) can be achieved using monomethoxytrityl (MMT) as the protecting group for the amino group of amino acid or polypeptide involved in ester formation, since ‘MMT’ is removable by mild acid treatment such as dichloroacetic acid that does not cleave a BOC group. After the amino group of the amino acid or polypeptide, forming an ester bond with hydroxyl of the drug, is demasked, the amino group is reacted with the activated form of a COOH group on PEG moiety of L2 (if present) under standard amide-forming conditions. In a preferred embodiment, L3 comprises a thiol-reactive group which links to a thiol group(s) of an antibody (or an antigen binding portion or other binding agent). The thiol-reactive group is optionally a maleimide or vinylsulfone, or bromoacetamide, or iodoacetamide, which links to a thiol group of the antibody. In some embodiments, the reagent bearing a thiol-reactive group is generated from succinimidyl-4-(N maleimidomethyl)cyclohexane-1-carboxylate (SMCC) or from succinimidyl-(epsilon-maleimido)caproate, for instance, with the thiol-reactive group being a maleimide group.

In another embodiments, m is 0, and AA comprises a peptide moiety, preferably a di, tri or tetrapeptide, that is cleavable by intracellular peptidase such as Cathepsin-B. Examples of cathepsin-B-cleavable peptides are: Phe-Lys, Val-Cit (Dubowchick, 2002), Ala-Leu, Leu-Ala-Leu, Ala-Leu-Ala-Leu (SEQ ID NO: 36) (Trouet et al., 1982), and Gly-Gly-Phe-Gly. (SEQ ID NO: 35) (See, e.g., WO2014/057687.)

In some embodiments, L1 is composed of intracellularly-cleavable peptide, such as cathepsin-B-cleavable peptide, connected to the collapsible unit, such as p-aminobenzyl alcohol (or p-amino-benzyloxycarbonyl) at the peptide's C-terminus, the benzyl alcohol portion of which is in turn directly attached to a hydroxyl group of the cytotoxic agent, in chloroformate form. In this embodiment, n is 0. Alternatively, when n is non-zero, the benzyl alcohol portion of the p-amidobenzyl alcohol (or p-amino-benzyloxycarbonyl) moiety is attached to the N-terminus of the amino acid or peptide linking at the hydroxyl group of the drug (e.g., cytotoxic agent) through the activated form of p-amidobenzyl alcohol, namely PABOCOPNP where PNP is p-nitrophenyl. In some embodiments, the linker comprises a thiol-reactive group which links to thiol groups of the antibody (or antigen binding portion or other binding agent). The thiol-reactive group is optionally a maleimide or vinylsulfone, or bromoacetamide, or iodoacetamide, which links to thiol groups of the antibody. In a preferred embodiment, the component bearing a thiol-reactive group is generated from succinimidyl-4-(N maleimidomethyl)cyclohexane-1-carboxylate (SMCC) or from succinimidyl-(epsilon-maleimido)caproate, for instance, with the thiol-reactive group being a maleimide group.

In some embodiments, where the drug is a cytotoxic agent such as a camptothecin or analog or derivative thereof having a 20-hydroxyl, L1 is composed of intracellularly-cleavable peptide, such as cathepsin-B-cleavable peptide, connected to the collapsible linker p-aminobenzyl alcohol (or p-amino-benzyloxycarbonyl) at the peptide's C-terminus, the benzyl alcohol portion of which is in turn directly attached to CPT-20-O-chloroformate. In this embodiment, n is 0. Alternatively, when n is non-zero, the benzyl alcohol portion of the p-amidobenzyl alcohol moiety is attached to the N-terminus of the amino acid or polypeptide linking at CPT's 20 position through the activated form of p-amidobenzyl alcohol, namely PABOCOPNP where PNP is p-nitrophenyl. In a preferred embodiment, the linker comprises a thiol-reactive group which links to thiol groups of an antibody (or antigen binding portion or other binding agent). The thiol-reactive group is optionally a maleimide or vinylsulfone, or bromoacetamide, or iodoacetamide, which links to thiol groups of an antibody. In a preferred embodiment, the component bearing a thiol-reactive group is generated from succinimidyl-4-(N maleimidomethyl)cyclohexane-1-carboxylate (SMCC) or from succinimidyl-(epsilon-maleimido)caproate, for instance, with the thiol-reactive group being a maleimide group.

In some embodiments, the L2 component of the conjugate is present and contains a polyethylene glycol (PEG) spacer that can be of up to about MW 5000 in size, and in a preferred embodiment, PEG is a defined PEG with (1-12 or 1-30) repeating monomeric units. In some embodiments, PEG is a defined PEG with 1-12 repeating monomeric units. The introduction of PEG may involve using heterobifunctionalized PEG derivatives which are available commercially. The heterobifunctional PEG typically contains an azide or acetylene group. An example of a heterobifunctional defined PEG containing 8 repeating monomeric units, with ‘NHS’ being succinimidyl, is given below in the following formula:

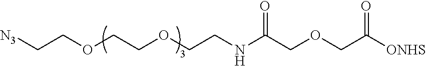

In some embodiments, L3 has a plurality of acetylene (or azide) groups, ranging from 2-40, but preferably 2-20, and more preferably 2-5, and a single antibody binding moiety.

A representative conjugate, in which the drug is a cytotoxic agent such as SN-38 (a CPT analog), prepared with a maleimide-containing SN-38-linker derivative, with the bonding to an antibody (designated MAb) represented as a succinimide, is given below. Here, m=0, and the 20-O-AA ester bonding to SN-38 is glycinate; azide-acetylene coupling joining of L2 and L3 results in the triazole moiety as shown.

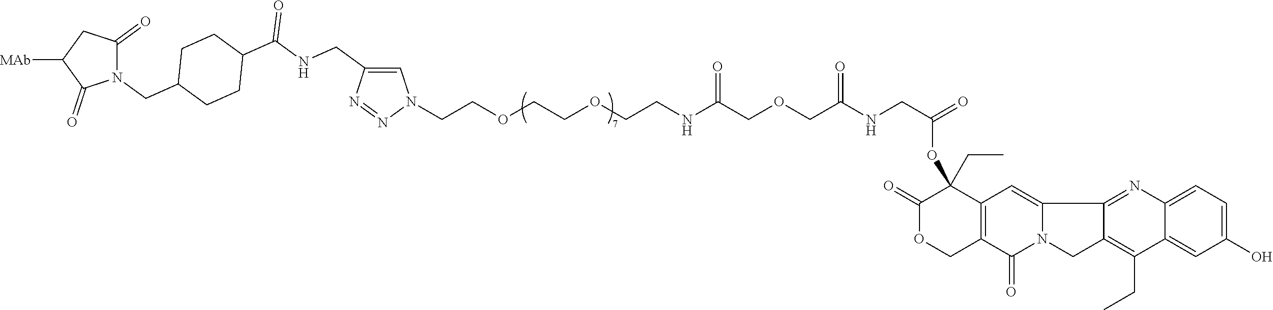

In another representative conjugate, prepared with a maleimide-containing SN-38-linker derivative, with the bonding to an antibody (MAb) represented as a succinimide, is shown below. Here, n=0 in the general formula 2; ‘L1’ contains a cathepsin-B-cleavable dipeptide, Phe-Lys, attached to the collapsible p-aminobenzyl alcohol moiety, and the latter is attached to SN-38 as a carbonate bonding at the 20 position; azide-acetylene coupling joining the ‘L2’ and ‘L3’ parts results in the triazole moiety as shown.

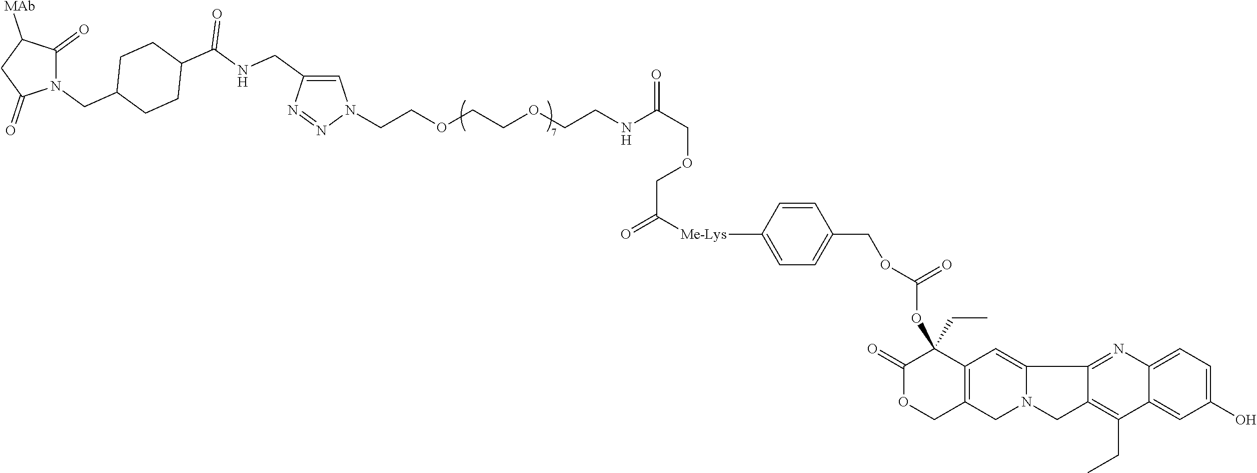

Another representative SN-38 conjugate, mAb-CL2-SN-38, is prepared with a maleimide-containing SN-38-linker derivative, with the bond to an antibody represented as a succinimide, is given below. Here, the 20-O-AA ester bonding to SN-38 is glycinate that is attached to L1 portion via a p-aminobenzyl alcohol moiety and a cathepsin-B-cleavable dipeptide; the latter is in turn attached to ‘L2’ via an amide bond, while ‘L2’ and ‘L3’ parts are coupled via azide-acetylene ‘click chemistry’.

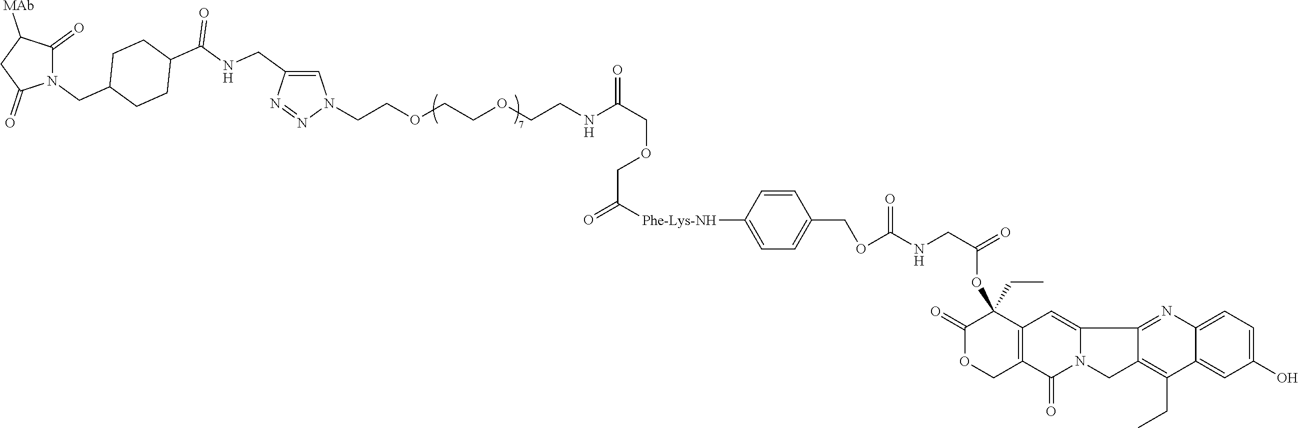

In another representative example, ‘L1’ contains a single amino acid attached to the collapsible p-aminobenzyl alcohol moiety, where the p-aminobenzyl alcohol is substituted or unsubstituted (R), where m=1 and n=0 in the general conjugate formula, Ab-[L3]-[L2]-[L1]ₘ-AAₙ-drug, and the drug is exemplified with SN-38. The structure is represented below (referred to as MAb-CLX-SN-38). Single amino acid of AA can be selected from any one of the following L-amino acids: alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine. The substituent R on 4-aminobenzyl alcohol moiety is hydrogen or an alkyl group selected from C1-C10 alkyl groups.

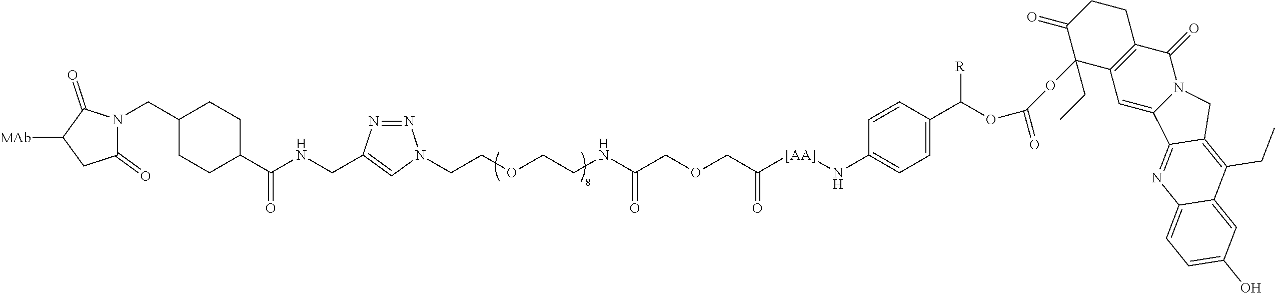

An embodiment of mAb-CLX-SN-38 (above), wherein the single amino acid AA is L-lysine and R=H, and the drug is a cytotoxic agent exemplified by SN-38 (referred to as mAb-CL2A-SN-38) is shown below:

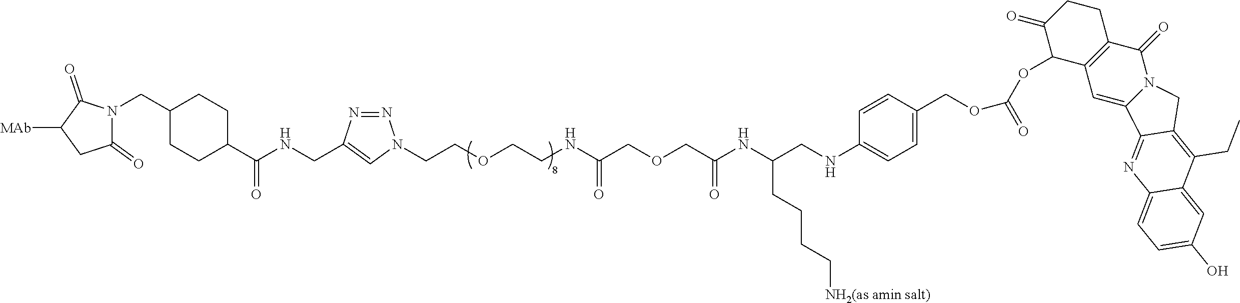

In other embodiments, a drug is a cytotoxic agent that is attached to a linker comprising a stretcher unit (Z) attached to an Amino Acid unit (AA) attached to a Spacer unit (Y), where the stretcher unit is attached to the antibody (or antigen binding portion thereof or other binding agent, designated Ab or MAb) and the Spacer unit is attached to an amino group of a cytotoxic agent. Such a linker has the following formula:

Ab-Z-AA-Y-cytotoxic agent,

where Z is selected from -(Succinimid-3-yl-N)—(CH2)ₙ²-C(═O)—, —CH₂—C(═O)—NH—(CH₂)n³-C(═O)—, —C(═O)-cycHex(1,4)-CH2-(N-Iy-3-diminiccuS)-, or —C(═O)—(CH2)n⁴-C(═O)—, wherein n² represents an integer of 2 to 8, n³ represents an integer of 1 to 8, and n⁴ represents an integer of 1 to 8; cyc.Hex(1,4) represents a 1,4-cyclohexylene group; and (N-Iv-3-diminiccuS)— has a structure represented by the following formula:

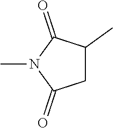

In some embodiments, AA is a peptide of from 2 to 7 amino acids. In some embodiments, the spacer unit Y is —NH—(CH₂)_{b}—(C═O)— or —NH—CH₂—O—CH₂—(C═O)—, where b is an integer from 1 to 5.

In some embodiments, the cytotoxic agent is exatecan. In some embodiments, the amino acid unit (AA) is -Gly-Gly-Phe-Gly-(SEQ ID NO: 35). In some embodiments, the spacer unit Y is —NH—CH₂—O—CH₂—(C═O)—.

In some embodiments, the linker-cytotoxic agent has the following structure:

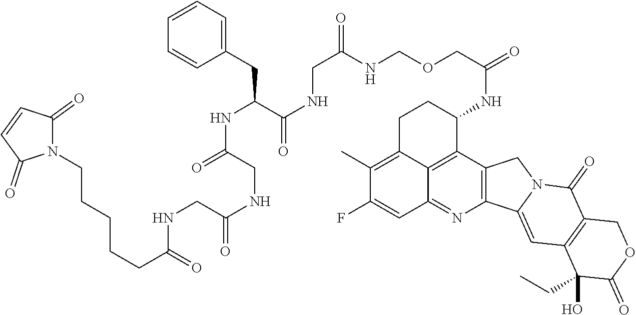 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)[C@H](Cc1ccccc1)NC(=O)CNC(=O)CNC(=O)CCCCCN1C(=O)C=CC1=O)CC3]

where the released cytotoxic agent is DXd (see U.S. Pat. No. 9,808,537).

### Attachment of Drug-Linkers to Antibodies, Antigen Binding Portions and Other Binding Agents

Techniques for attaching drugs to antibodies (or antigen binding portions thereof or other binding agents) via linkers are well-known in the art. See, e.g., Alley et al., Current Opinion in Chemical Biology 2010 14:1-9; Senter, Cancer J., 2008, 14(3):154-169. In some embodiments, a linker is first attached to a drug (e.g., a cytotoxic agent(s)) and then the drug-linker is attached to the antibody or antigen binding portion thereof or other binding agent. In some embodiments, a linker is first attached to an antibody or antigen binding portion thereof or other binding agent, and then a drug is attached to the linker. In the following discussion, the term drug-linker is used to exemplify attachment of linkers or drug-linkers to antibodies or antigen binding portions thereof or other binding agents; the skilled artisan will appreciate that the selected attachment method can be determined according to linker and the cytotoxic agent or other drug. In some embodiments, a drug is attached to an antibody or antigen binding portion thereof or other binding agent via a linker in a manner that reduces the activity of the drug until it is released from the conjugate (e.g., by hydrolysis, by proteolytic degradation or by a cleaving agent.).

Generally, a conjugate may be prepared by several routes employing organic chemistry reactions, conditions, and reagents known to those skilled in the art, including: (1) reaction of a nucleophilic group of an antibody (or antigen binding portion thereof or other binding agent) with a bivalent linker reagent to form an antibody-linker intermediate via a covalent bond, followed by reaction with a drug (e.g., a cytotoxic agent); and (2) reaction of a nucleophilic group of a drug (e.g., a cytotoxic agent) with a bivalent linker reagent, to form drug-linker, via a covalent bond, followed by reaction with a nucleophilic group of an antibody or antigen binding portion thereof or other binding agent. Exemplary methods for preparing conjugates via the latter route are described in U.S. Pat. No. 7,498,298, which is expressly incorporated herein by reference.

Nucleophilic groups on antibodies, antigen binding portions and other binding agents include, but are not limited to: (i) N-terminal amine groups, (ii) side chain amine groups, e.g. lysine, (iii) side chain thiol groups, e.g. cysteine, and (iv) sugar hydroxyl or amino groups where the antibody is glycosylated. Amine, thiol, and hydroxyl groups are nucleophilic and capable of reacting to form covalent bonds with electrophilic groups on linker moieties and linker reagents including: (i) active esters such as NHS esters, HOBt esters, haloformates, and acid halides; (ii) alkyl and benzyl halides such as haloacetamides; and (iii) aldehydes, ketones, carboxyl, and maleimide groups. Certain antibodies (antigen binding portions and other binding agents) have reducible interchain disulfides, i.e., cysteine bridges. Antibodies (and antigen binding portions and other binding agents) may be made reactive for conjugation with linker reagents by treatment with a reducing agent such as DTT (dithiothreitol) or tricarbonylethylphosphine (TCEP), such that the antibody is fully or partially reduced. Each cysteine bridge will thus form, theoretically, two reactive thiol nucleophiles. Additional nucleophilic groups can be introduced into antibodies (and antigen binding portions and other binding agents) through modification of lysine residues, e.g., by reacting lysine residues with 2-iminothiolane (Traut's reagent), resulting in conversion of an amine into a thiol. Reactive thiol groups may also be introduced into an antibody (and antigen binding portions and other binding agents) by introducing one, two, three, four, or more cysteine residues (e.g., by preparing antibodies, antigen binding portions and other binding agents comprising one or more non-native cysteine amino acid residues).

Conjugates may also be produced by reaction between an electrophilic group on an antibody (or antigen binding portion thereof or other binding agent), such as an aldehyde or ketone carbonyl group, with a nucleophilic group on a linker reagent or drug. Useful nucleophilic groups on a linker reagent include, but are not limited to, hydrazide, oxime, amino, hydrazine, thiosemicarbazone, hydrazine carboxylate, and arylhydrazide. In an embodiment, an antibody (or antigen binding portion thereof or other binding agent) is modified to introduce electrophilic moieties that are capable of reacting with nucleophilic substituents on the linker reagent or drug. In another embodiment, the sugars of glycosylated antibodies may be oxidized, e.g. with periodate oxidizing reagents, to form aldehyde or ketone groups which may react with the amine group of linker reagents or drug moieties. The resulting imine Schiff base groups may form a stable linkage, or may be reduced, e.g., by borohydride reagents to form stable amine linkages. In one embodiment, reaction of the carbohydrate portion of a glycosylated antibody with either galactose oxidase or sodium meta-periodate may yield carbonyl (aldehyde and ketone) groups in the antibody (or antigen binding portion thereof or other binding agent) that can react with appropriate groups on the drug (see, e.g., Hermanson, Bioconjugate Techniques). In another embodiment, antibodies containing N-terminal serine or threonine residues can react with sodium meta-periodate, resulting in production of an aldehyde in place of the first amino acid (Geoghegan & Stroh, (1992) Bioconjugate Chem. 3:138-146; U.S. Pat. No. 5,362,852). Such an aldehyde can be reacted with a cytotoxic agent or linker.

Exemplary nucleophilic groups on a drug, such as a cytotoxic agent, include, but are not limited to: amine, thiol, hydroxyl, hydrazide, oxime, hydrazine, thiosemicarbazone, hydrazine carboxylate, and arylhydrazide groups capable of reacting to form covalent bonds with electrophilic groups on linker moieties and linker reagents including: (i) active esters such as NHS esters, HOBt esters, haloformates, and acid halides; (ii) alkyl and benzyl halides such as haloacetamides; (iii) aldehydes, ketones, carboxyl, and maleimide groups.

Nonlimiting exemplary cross-linkers that may be used to prepare a conjugate are described herein or are known to persons of ordinary skill in the art. Methods of using such cross-linkers to link two moieties, including an antibody (or antigen binding portion or other binding agent) and a chemical moiety, are known in the art. In some embodiments, a fusion protein comprising an antibody and a drug may be made, e.g., by recombinant techniques or peptide synthesis. A recombinant DNA molecule may comprise regions encoding the antibody (or antigen binding portion thereof or other binding agent) and active portions (e.g., cytotoxic portions) of the conjugate either adjacent to one another or separated by a region encoding a linker which does not destroy the desired properties of the conjugate.

In some embodiments, a drug-linker is attached to an interchain cysteine residue(s) of an antibody (or antigen binding portion thereof or other binding agent). See, e.g., WO2004/010957 and WO2005/081711. In such embodiments, the linker typically comprises a maleimide group for attachment to the cysteine residues of an interchain disulfide. In some embodiments, the linker or drug-linker is attached to a cysteine residue(s) of an antibody or antigen binding portion thereof as described in U.S. Pat. No. 7,585,491 or 8,080250. The drug loading of the resulting conjugate typically ranges from 1 to 8.

In some embodiments, the linker or drug-linker is attached to a lysine or cysteine residue(s) of an antibody (or antigen binding portion thereof or other binding agent) as described in WO2005/037992 or WO2010/141566. The drug loading of the resulting conjugate typically ranges from 1 to 8.

In some embodiments, engineered cysteine residues, poly-histidine sequences, glycoengineering tags, or transglutaminase recognition sequences can be used for site-specific attachment of linkers or drug-linkers to antibodies or antigen binding portions thereof or other binding agents.

In some embodiments, a drug-linker(s) is attached to an engineered cysteine residue at an Fc residue other than an interchain disulfide. In some embodiments, a drug-linker(s) is attached to an engineered cysteine introduced into an IgG (typically an IgG1) at position 118, 221, 224, 227, 228, 230, 231, 223, 233, 234, 235, 236, 237, 238, 239, 240, 241, 243, 244, 245, 247, 249, 250, 258, 262, 263, 264, 265, 266, 267, 268, 269, 270, 271, 272, 273, 275, 276, 278, 280, 281, 283, 285, 286, 291, 292, 293, 294, 295, 296, 297, 298, 299, 300, 302, 305, 313, 318, 323, 324, 325, 327, 328, 329, 330, 331, 332, 333, 335, 336, 396, and/or 428, of the heavy chain and/or to a light chain at position 106, 108, 142 (light chain), 149 (light chain), and/or position V205, according to the EU numbering of Kabat. An exemplary substitution for site specific conjugation using an engineered cysteine is S239C (see, e.g., US 20100158909; numbering of the Fc region is according to the EU index).

In some embodiments, a linker or drug-linker(s) is attached to one or more introduced cysteine residues of an antibody (or antigen binding portion thereof or other binding agent) as described in WO2006/034488, WO2011/156328 and/or WO2016040856.

In some embodiments, an exemplary substitution for site specific conjugation using bacterial transglutaminase is N297S or N297Q of the Fc region. In some embodiments, a linker or drug-linker(s) is attached to the glycan or modified glycan of an antibody or antigen binding portion or a glycoengineered antibody (or other binding agent). See, e.g., WO2017/147542, WO2020/123425, WO2020/245229, WO2014/072482; WO2014//065661, WO2015/057066 and WO2016/022027; the disclosure of which are incorporated by reference herein.

### Pharmaceutical Formulations

Other aspects of the CD70 antibodies and antigen binding portions thereof or other binding agents and conjugates of any of these relate to compositions comprising active ingredients (i.e., including a CD70 antibody or antigen-binding portion thereof or other binding agent or conjugate thereof as described herein or a nucleic acid encoding an antibody or antigen-binding portion thereof or other binding agent as described herein). In some embodiments, the composition is a pharmaceutical composition. As used herein, the term “pharmaceutical composition” refers to an active agent in combination with a pharmaceutically acceptable carrier accepted for use in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The preparation of a pharmacological composition that contains active ingredients dissolved or dispersed therein is well understood in the art and need not be limited based on any particular formulation. Typically such compositions are prepared as injectable either as liquid solutions or suspensions; however, solid forms suitable for rehydration, or suspensions, in liquid prior to use can also be prepared. A preparation can also be emulsified or presented as a liposome composition. A CD70 antibody or antigen binding portion thereof or other binding agent or conjugate thereof can be mixed with excipients that are pharmaceutically acceptable and compatible with the active ingredient and in amounts suitable for use in the therapeutic methods described herein. Suitable excipients are, for example, water, saline, dextrose, glycerol, ethanol or the like and combinations thereof. In addition, if desired, a pharmaceutical composition can contain minor amounts of auxiliary substances such as wetting or emulsifying agents, pH buffering agents and the like which enhance or maintain the effectiveness of the active ingredient (e.g., a CD70 antibody or antigen binding portion thereof or other binding agent or conjugate thereof). The pharmaceutical compositions as described herein can include pharmaceutically acceptable salts of the components therein. Pharmaceutically acceptable salts include the acid addition salts (formed with the free amino groups of a polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that contain the active ingredients (e.g., a CD70 antibody and/or antigen binding portions thereof or other binding agent or conjugate thereof) and water, and may contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active agent that will be effective in the treatment of a particular disorder or condition will depend on the nature of the disorder or condition, and can be determined by standard clinical techniques.

In some embodiments, a pharmaceutical composition comprising a CD70 antibody or antigen-binding portion thereof or other binding agent conjugate thereof as described herein or a nucleic acid encoding a CD70 antibody or antigen-binding portion thereof or other binding agent as described herein can be a lyophilisate.

In some embodiments, a syringe comprising a therapeutically effective amount of a CD70 antibody or antigen binding portion thereof or conjugate thereof, or a pharmaceutical composition described herein is provided.

### Treatment of Cancer

In some embodiments, the CD70 antibodies or antigen binding portions thereof, other binding agents and conjugates as described herein can be used in a method(s) comprising administering a CD70 antibody or antigen-binding portion thereof or other binding agent or conjugate thereof as described herein to a subject in need thereof, such as a subject having cancer.

In some embodiments, provided are methods of treating cancer comprising administering a CD70 antibody or antigen-binding portion thereof or other binding agent or conjugate thereof comprising a heavy chain variable region (VH) and a light chain variable region (VL), the VH and VL regions having amino acid sequences set forth in the pairs of amino acid sequences selected from SEQ ID NO:3 and SEQ ID NO:4, respectively; SEQ ID NO:5 and SEQ ID NO:6, respectively; SEQ ID NO:7 and SEQ ID NO:8, respectively; SEQ ID NO:9 and SEQ ID NO:10, respectively; and SEQ ID NO:11 and SEQ ID NO:12; respectively. In some embodiments, provided are methods of treating cancer comprising administering a CD70 antibody or antigen-binding portion thereof or other binding agent or conjugate thereof comprising a heavy chain variable region (VH) and a light chain variable region (VL), the VH and VL regions having amino acid sequences set forth in SEQ ID NO:3 and SEQ ID NO:4, respectively. In some embodiments, provided are methods of treating cancer comprising administering a CD70 antibody or antigen-binding portion thereof or other binding agent or conjugate thereof comprising a heavy chain variable region (VH) and a light chain variable region (VL), the VH and VL regions having amino acid sequences set forth in SEQ ID NO:5 and SEQ ID NO:6, respectively. In some embodiments, provided are methods of treating cancer comprising administering a CD70 antibody or antigen-binding portion thereof or other binding agent or conjugate thereof comprising a heavy chain variable region (VH) and a light chain variable region (VL), the VH and VL regions having amino acid sequences set forth in SEQ ID NO:7 and SEQ ID NO:8, respectively. In some embodiments, provided are methods of treating cancer comprising administering a CD70 antibody or antigen-binding portion thereof or other binding agent or conjugate thereof comprising a heavy chain variable region (VH) and a light chain variable region (VL), the VH and VL regions having amino acid sequences set forth in SEQ ID NO:9 and SEQ ID NO:10, respectively. In some embodiments, provided are methods of treating cancer comprising administering a CD70 antibody or antigen-binding portion thereof or other binding agent or conjugate thereof comprising a heavy chain variable region (VH) and a light chain variable region (VL), the VH and VL regions having amino acid sequences set forth in and SEQ ID NO: 11 and SEQ ID NO:12; respectively.

In some embodiments, provided are methods of treating cancer comprising administering a CD70 antibody or antigen-binding portion thereof or other binding agent or conjugate thereof comprising a heavy chain variable region (VH) and a light chain variable region (VL), the VH and VL regions having amino acid sequences set forth in the pairs of amino acid sequences selected from SEQ ID NO:3 and SEQ ID NO:4, respectively; SEQ ID NO:5 and SEQ ID NO:6, respectively; SEQ ID NO:7 and SEQ ID NO:8, respectively; SEQ ID NO:9 and SEQ ID NO:10, respectively; and SEQ ID NO:11 and SEQ ID NO:12; respectively; wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified. In some embodiments, provided are methods of treating cancer comprising administering a CD70 antibody or antigen-binding portion thereof or other binding agent or conjugate thereof comprising a heavy chain variable region (VH) and a light chain variable region (VL), the VH and VL regions having amino acid sequences set forth in SEQ ID NO:3 and SEQ ID NO:4, respectively; wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified. In some embodiments, provided are methods of treating cancer comprising administering a CD70 antibody or antigen-binding portion thereof or other binding agent or conjugate thereof comprising a heavy chain variable region (VH) and a light chain variable region (VL), the VH and VL regions having amino acid sequences set forth in SEQ ID NO:5 and SEQ ID NO:6, respectively; wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified. In some embodiments, provided are methods of treating cancer comprising administering a CD70 antibody or antigen-binding portion thereof or other binding agent or conjugate thereof comprising a heavy chain variable region (VH) and a light chain variable region (VL), the VH and VL regions having amino acid sequences set forth in SEQ ID NO:7 and SEQ ID NO:8, respectively; wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified. In some embodiments, provided are methods of treating cancer comprising administering a CD70 antibody or antigen-binding portion thereof or other binding agent or conjugate thereof comprising a heavy chain variable region (VH) and a light chain variable region (VL), the VH and VL regions having amino acid sequences set forth in SEQ ID NO:9 and SEQ ID NO:10, respectively; wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified. In some embodiments, provided are methods of treating cancer comprising administering a CD70 antibody or antigen-binding portion thereof or other binding agent or conjugate thereof comprising a heavy chain variable region (VH) and a light chain variable region (VL), the VH and VL regions having amino acid sequences set forth in SEQ ID NO:11 and SEQ ID NO:12; respectively; wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified.

In some embodiments, provided are methods of treating cancer comprising administering a CD70 antibody or antigen-binding portion thereof or other binding agent or conjugate thereof comprising a heavy chain variable region (VH) and a light chain variable region (VL), the VH and VL regions having the amino acid sequences set forth in the pairs of amino acid sequences selected from SEQ ID NO:3 and SEQ ID NO:4, respectively; SEQ ID NO:5 and SEQ ID NO:6, respectively; SEQ ID NO:7 and SEQ ID NO:8, respectively; SEQ ID NO:9 and SEQ ID NO:10, respectively; and SEQ ID NO:11 and SEQ ID NO:12; respectively; wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified. In some embodiments, provided are methods of treating cancer comprising administering a CD70 antibody or antigen-binding portion thereof or other binding agent or conjugate thereof comprising a heavy chain variable region (VH) and a light chain variable region (VL), the VH and VL regions having the amino acid sequences set forth in SEQ ID NO:3 and SEQ ID NO:4, respectively; wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified. In some embodiments, provided are methods of treating cancer comprising administering a CD70 antibody or antigen-binding portion thereof or other binding agent or conjugate thereof comprising a heavy chain variable region (VH) and a light chain variable region (VL), the VH and VL regions having the amino acid sequences set forth in SEQ ID NO:5 and SEQ ID NO:6, respectively; wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified. In some embodiments, provided are methods of treating cancer comprising administering a CD70 antibody or antigen-binding portion thereof or other binding agent or conjugate thereof comprising a heavy chain variable region (VH) and a light chain variable region (VL), the VH and VL regions having the amino acid sequences set forth in SEQ ID NO:7 and SEQ ID NO:8, respectively; wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified. In some embodiments, provided are methods of treating cancer comprising administering a CD70 antibody or antigen-binding portion thereof or other binding agent or conjugate thereof comprising a heavy chain variable region (VH) and a light chain variable region (VL), the VH and VL regions having the amino acid sequences set forth in SEQ ID NO:9 and SEQ ID NO:10, respectively; wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified. In some embodiments, provided are methods of treating cancer comprising administering a CD70 antibody or antigen-binding portion thereof or other binding agent or conjugate thereof comprising a heavy chain variable region (VH) and a light chain variable region (VL), the VH and VL regions having the amino acid sequences set forth in SEQ ID NO: 11 and SEQ ID NO:12; respectively; wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified.

In some embodiments, provided are methods of treating cancer comprising administering a CD70 antibody or antigen-binding portion thereof or other binding agent or conjugate thereof comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having the amino acids sequences set forth in the sets of amino acid sequences selected from (i) SEQ ID NO:21, SEQ ID NO:22, SEQ ID NO:13, SEQ ID NO:24, SEQ ID NO:25 and SEQ ID NO:26, respectively; (ii) SEQ ID NO:21, SEQ ID NO:22, SEQ ID NO:14, SEQ ID NO:24, SEQ ID NO:25 and SEQ ID NO:26, respectively; (iii) SEQ ID NO:21, SEQ ID NO:22, SEQ ID NO:15, SEQ ID NO:24, SEQ ID NO:25 and SEQ ID NO:26, respectively; (iv) SEQ ID NO:21, SEQ ID NO:22, SEQ ID NO:23, SEQ ID NO:24, SEQ ID NO:25 and SEQ ID NO:18, respectively; and (v) SEQ ID NO:16, SEQ ID NO:17, SEQ ID NO:23, SEQ ID NO:24, SEQ ID NO:25 and SEQ ID NO:26; respectively. In some embodiments, each VH and VL region comprises a humanized framework region. In some embodiments, each VH and VL region comprises a human framework region.

In some embodiments, provided are methods of treating cancer comprising administering a CD70 antibody or antigen-binding portion thereof or other binding agent or conjugate thereof comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having the amino acids sequences set forth in SEQ ID NO:21, SEQ ID NO:22, SEQ ID NO:13, SEQ ID NO:24, SEQ ID NO:25 and SEQ ID NO:26, respectively. In some embodiments, each VH and VL region comprises a humanized framework region. In some embodiments, each VH and VL region comprises a human framework region.

In some embodiments, provided are methods of treating cancer comprising administering a CD70 antibody or antigen-binding portion thereof or other binding agent or conjugate thereof comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having the amino acids sequences set forth in SEQ ID NO:21, SEQ ID NO:22, SEQ ID NO:14, SEQ ID NO:24, SEQ ID NO:25 and SEQ ID NO:26, respectively. In some embodiments, each VH and VL region comprises a humanized framework region. In some embodiments, each VH and VL region comprises a human framework region.

In some embodiments, provided are methods of treating cancer comprising administering a CD70 antibody or antigen-binding portion thereof or other binding agent or conjugate thereof comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having the amino acids sequences set forth in SEQ ID NO:21, SEQ ID NO:22, SEQ ID NO:15, SEQ ID NO:24, SEQ ID NO:25 and SEQ ID NO:26, respectively. In some embodiments, each VH and VL region comprises a humanized framework region. In some embodiment of treating cancer, each VH and VL region comprises a human framework region.

In some embodiments, provided are methods of treating cancer comprising administering a CD70 antibody or antigen-binding portion thereof or other binding agent or conjugate thereof comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having the amino acids sequences set forth in SEQ ID NO:21, SEQ ID NO:22, SEQ ID NO:23, SEQ ID NO:24, SEQ ID NO:25 and SEQ ID NO:18, respectively. In some embodiments, each VH and VL region comprises a humanized framework region. In some embodiments, each VH and VL region comprises a human framework region.

In some embodiments, provided are methods of treating cancer comprising administering a CD70 antibody or antigen-binding portion thereof or other binding agent or conjugate thereof comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having the amino acids sequences set forth in SEQ ID NO:16, SEQ ID NO:17, SEQ ID NO:23, SEQ ID NO:24, SEQ ID NO:25 and SEQ ID NO:26; respectively. In some embodiments, each VH and VL region comprises a humanized framework region. In some embodiments, each VH and VL region comprises a human framework region.

In some embodiments, the subject is in need of treatment for a cancer and/or a malignancy. In some embodiments, the subject is in need of treatment for a CD70+ cancer or a CD70+ malignancy, such as for example, hepatocellular cancer, colorectal cancer, pancreatic cancer, ovarian cancer, indolent Non-Hodgkin's Lymphoma (indolent NHLs) (e.g., follicular NHLs, small lymphocytic lymphomas, lymphoplasmacytic NHLs, or marginal zone NHLs), Non-Hodgkin's Lymphoma (non-indolent), cancers of the B-cell lineage, including, e.g., Burkitt's lymphoma and chronic lymphocytic leukemia, multiple myeloma, renal cell cancers, nasopharyngeal cancers, thymic cancers and gliomas. In some embodiments, the method is for treating a subject having a CD70+ cancer or malignancy. In some embodiments, the method is for treating hepatocellular cancer in a subject. In some embodiments, the method is for treating colorectal cancer in a subject. In some embodiments, the method is for treating pancreatic cancer in a subject. In some embodiments, the method is for treating ovarian cancer in a subject. In some embodiments, the method is for treating an indolent Non-Hodgkin's Lymphoma (indolent NHLs), such as for example a follicular NHL, a small lymphocytic lymphoma, a lymphoplasmacytic NHL, or a marginal zone NHL in a subject. In some embodiments, the method is for treating Non-Hodgkin's Lymphoma in a subject. In some embodiments, the method is for treating cancers of the B-cell lineage, such as, for example, Burkitt's lymphoma or chronic lymphocytic leukemia, in a subject. In some embodiments, the method is for treating multiple myeloma in a subject. In some embodiments, the method is for treating renal cell cancer in a subject. In some embodiments, the method is for treating nasopharyngeal carcinoma in a subject. In some embodiments, the method is for treating thymic cancer in a subject. In some embodiments, the method is for treating a glioma in a subject.

The methods described herein include administering a therapeutically effective amount of a CD70 binding antibody or antigen binding portion thereof or other binding agent or conjugate thereof to a subject having a CD70+ cancer or malignancy. As used herein, the phrases “therapeutically effective amount”, “effective amount” or “effective dose” refer to an amount of the CD70 antibody or antigen binding portion thereof or other binding agent or conjugate as described herein that provides a therapeutic benefit in the treatment of, management of or prevention of relapse of a cancer or malignancy, e.g., an amount that provides a statistically significant decrease in at least one symptom, sign, or marker of a tumor or malignancy. Determination of a therapeutically effective amount is well within the capability of those skilled in the art. Generally, a therapeutically effective amount can vary with the subject's history, age, condition, sex, as well as the severity and type of the medical condition in the subject, and administration of other pharmaceutically active agents.

The terms “cancer” and “malignancy” refer to an uncontrolled growth of cells which interferes with the normal functioning of the bodily organs and systems. A cancer or malignancy may be primary or metastatic, i.e. that is it has become invasive, seeding tumor growth in tissues remote from the original tumor site. A “tumor” refers to an uncontrolled growth of cells which interferes with the normal functioning of the bodily organs and systems. A subject that has a cancer is a subject having objectively measurable cancer cells present in the subject's body. Included in this definition are benign tumors and malignant cancers, as well as potentially dormant tumors and micro-metastases. Cancers that migrate from their original location and seed other vital organs can eventually lead to the death of the subject through the functional deterioration of the affected organs. Hematologic malignancies (hematopoietic cancers), such as leukemias and lymphomas, are able to, for example, out-compete the normal hematopoietic compartments in a subject, thereby leading to hematopoietic failure (in the form of anemia, thrombocytopenia and neutropenia) ultimately causing death.

Examples of cancers include, but are not limited to, carcinomas, lymphomas, blastomas, sarcomas, and leukemias. More particular examples of such cancers include, but are not limited to, basal cell cancer, biliary tract cancer, bladder cancer, bone cancer, brain and CNS cancer, breast cancer (e.g., triple negative breast cancer), cancer of the peritoneum, cervical cancer; cholangiocarcinoma, choriocarcinoma, chondrosarcoma, colon and rectum cancer (colorectal cancer), connective tissue cancer, cancer of the digestive system, endometrial cancer, esophageal cancer, eye cancer, cancer of the head and neck, gastric cancer (including gastrointestinal cancer and stomach cancer), glioblastoma (GBM), hepatic cancer, hepatoma, intra-epithelial neoplasm, kidney or renal cancer (e.g., clear cell cancer), larynx cancer, leukemia, liver cancer, lung cancer (e.g., small-cell lung cancer, non-small cell lung cancer, adenocarcinoma of the lung, and squamous cancer of the lung), lymphoma including Hodgkin's and non-Hodgkin's lymphoma, melanoma, mesothelioma, myeloma, neuroblastoma, oral cavity cancer (e.g., lip, tongue, mouth, and pharynx), ovarian cancer, pancreatic cancer, prostate cancer, retinoblastoma, rhabdomyosarcoma, cancer of the respiratory system, salivary gland cancer, sarcoma, skin cancer, squamous cell cancer, testicular cancer, thyroid cancer, uterine or endometrial cancer, uterine serious cancer, cancer of the urinary system, vulval cancer; as well as other carcinomas and sarcomas, as well as B-cell lymphoma (including low grade/follicular non-Hodgkin's lymphoma (NHL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL, mantle cell lymphoma, AIDS-related lymphoma, and Waldenstrom's Macroglobulinemia), chronic lymphocytic leukemia (CLL), acute lymphoblastic leukemia (ALL), Hairy cell leukemia, chronic myeloblastic leukemia, and post-transplant lymphoproliferative disorder (PTLD), as well as abnormal vascular proliferation associated with phakomatoses, edema (such as that associated with brain tumors), and Meigs' syndrome.

In some embodiments, the cancer is a solid tumor. In some embodiments, the cancer is selected from a solid tumor, including but not limited to, hepatocellular cancer, colorectal cancer, renal cell carcer, pancreatic cancer, ovarian cancer, nasopharyngeal carcinoma, thymic cancer and gliomas. In some embodiments, the cancer is selected from a hematologic cancer, also referred to as a hematologic malignancy. In some embodiments, the cancer is selected from a hematologic cancer, such as indolent Non-Hodgkin's Lymphoma (indolent NHLs) (e.g., follicular NHLs, small lymphocytic lymphomas, lymphoplasmacytic NHLs, or marginal zone NHLs), Non-Hodgkin's Lymphoma (non-indolent), NS cancers of the B-cell lineage, including, e.g., Burkitt's lymphoma and chronic lymphocytic leukemia. In some embodiments, the cancer or malignancy is CD70-positive (CD70+). As used herein, the terms “CD70-positive” or “CD70+” are used to describe a cancer cell, a cluster of cancer cells, a tumor mass, or a metastatic cell that express CD70 on the cell surface (membrane-bound CD70). Some non-limiting examples of CD70-positive cancers include, for example, hepatocellular cancer, colorectal cancer, pancreatic cancer, ovarian cancer, indolent Non-Hodgkin's Lymphoma (indolent NHLs) (e.g., follicular NHLs, small lymphocytic lymphomas, lymphoplasmacytic NHLs, or marginal zone NHLs), Non-Hodgkin's Lymphoma, cancers of the B-cell lineage, including, e.g., Burkitt's lymphoma and chronic lymphocytic leukemia, multiple myeloma, renal cell cancers, nasopharyngeal cancers, thymic cancers and gliomas.

It is contemplated that the methods herein reduce tumor size or tumor burden in the subject, and/or reduce metastasis in the subject. In various embodiments, tumor size in the subject is decreased by about 25-50%, about 40-70% or about 50-90% or more. In various embodiments, the methods reduce the tumor size by 10%, 20%, 30% or more. In various embodiments, the methods reduce tumor size by 10%, 15%, 20%, 25%, 30%, 35%, 40%, 45%, 50%, 55%, 60%, 65%, 70%, 75%, 80%, 85%, 90%, 95% or 100%.

As used herein, a “subject” refers to a human or animal. Usually the animal is a vertebrate such as a primate, rodent, domestic animal or game animal. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In certain embodiments, the subject is a mammal, e.g., a primate, e.g., a human. The terms, “patient”, “individual” and “subject” are used interchangeably herein.

Preferably, the subject is a mammal. The mammal can be a human, non-human primate, mouse, rat, dog, cat, horse, or cow, but are not limited to these examples. Mammals other than humans can be advantageously used, for example, as subjects that represent animal models of, for example, various cancers. In addition, the methods described herein can be used to treat domesticated animals and/or pets. A subject can be male or female. In certain embodiments, the subject is a human.

In some embodiments, a subject can be one who has been previously diagnosed with or identified as suffering from a CD70+ cancer and in need of treatment, but need not have already undergone treatment for the CD70+ cancer. In some embodiments, a subject can also be one who has not been previously diagnosed as having a CD70+ cancer in need of treatment. In some embodiments, a subject can be one who exhibits one or more risk factors for a condition or one or more complications related to a CD70+ cancer or a subject who does not exhibit risk factors. A “subject in need” of treatment for a CD70+ cancer particular can be a subject having that condition or diagnosed as having that condition. In other embodiments, a subject “at risk of developing” a condition refers to a subject diagnosed as being at risk for developing the condition or at risk for having the condition again (e.g., a CD70+ cancer).

As used herein, the terms “treat,” “treatment,” “treating,” or “amelioration” when used in reference to a disease, disorder or medical condition, refer to therapeutic treatments for a condition, wherein the object is to reverse, alleviate, ameliorate, inhibit, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a condition is reduced or halted. That is, “treatment” includes not just the improvement of symptoms or markers, but also a cessation or at least slowing of progress or worsening of symptoms that would be expected in the absence of treatment. Beneficial or desired clinical results include, but are not limited to, reduction in CD70+ cancer cells in the subject, alleviation of one or more symptom(s), diminishment of extent of the deficit, stabilized (i.e., not worsening) state of a cancer or malignancy, delay or slowing of tumor growth and/or metastasis, and an increased lifespan as compared to that expected in the absence of treatment. As used herein, the term “administering,” refers to providing a CD70 binding antibody or antigen-binding portion thereof or other binding agent or conjugate as described herein or a nucleic acid encoding the CD70 antibody or antigen-binding portion thereof or other binding agent as described herein into a subject by a method or route which results in binding to the CD70 binding antibody or antigen binding portion thereof or other binding agent or conjugate to CD70+ cancer cells or malignant cells. Similarly, a pharmaceutical composition comprising a CD70 binding antibody or antigen-binding portion thereof or other binding agent or conjugate as described herein or a nucleic acid encoding the CD70 antibody or antigen-binding portion thereof or other binding agent as described herein disclosed herein can be administered by any appropriate route which results in an effective treatment in the subject.

The dosage ranges for a CD70 binding antibody or antigen binding portion thereof or binding agent or conjugate depend upon the potency, and encompass amounts large enough to produce the desired effect e.g., slowing of tumor growth or a reduction in tumor size. The dosage should not be so large as to cause unacceptable adverse side effects. Generally, the dosage will vary with the age, condition, and sex of the subject and can be determined by one of skill in the art. The dosage can also be adjusted by the individual physician in the event of any complication. In some embodiments, the dosage ranges from 0.1 mg/kg body weight to 10 mg/kg body weight. In some embodiments, the dosage ranges from 0.5 mg/kg body weight to 15 mg/kg body weight. In some embodiments, the dose range is from 0.5 mg/kg body weight to 5 mg/kg body weight. Alternatively, the dose range can be titrated to maintain serum levels between 1 ug/mL and 1000 ug/mL. For systemic administration, subjects can be administered a therapeutic amount, such as, e.g. 0.1 mg/kg, 0.5 mg/kg, 1.0 mg/kg, 2.0 mg/kg, 2.5 mg/kg, 5 mg/kg, 10 mg/kg, 12 mg/kg or more.

Administration of the doses recited above can be repeated. In a preferred embodiment, the doses recited above are administered weekly, biweekly, every three weeks or monthly for several weeks or months. The duration of treatment depends upon the subject's clinical progress and responsiveness to treatment.

In some embodiments, a dose can be from about 0.1 mg/kg to about 100 mg/kg. In some embodiments, a dose can be from about 0.1 mg/kg to about 25 mg/kg. In some embodiments, a dose can be from about 0.1 mg/kg to about 20 mg/kg. In some embodiments, a dose can be from about 0.1 mg/kg to about 15 mg/kg. In some embodiments, a dose can be from about 0.1 mg/kg to about 12 mg/kg. In some embodiments, a dose can be from about 1 mg/kg to about 100 mg/kg. In some embodiments, a dose can be from about 1 mg/kg to about 25 mg/kg. In some embodiments, a dose can be from about 1 mg/kg to about 20 mg/kg. In some embodiments, a dose can be from about 1 mg/kg to about 15 mg/kg. In some embodiments, a dose can be from about 1 mg/kg to about 12 mg/kg. In some embodiments, a dose can be from about 1 mg/kg to about 10 mg/kg.

In some embodiments, a dose can be administered intravenously. In some embodiments, an intravenous administration can be an infusion occurring over a period of from about 10 minutes to about 4 hours. In some embodiments, an intravenous administration can be an infusion occurring over a period of from about 30 minutes to about 90 minutes.

In some embodiments, a dose can be administered weekly. In some embodiments, a dose can be administered bi-weekly. In some embodiments, a dose can be administered about every 2 weeks. In some embodiments, a dose can be administered about every 3 weeks. In some embodiments, a dose can be administered every four weeks.

In some embodiments, a total of from about 2 to about 10 doses are administered to a subject. In some embodiments, a total of 4 doses are administered. In some embodiments, a total of 5 doses are administered. In some embodiments, a total of 6 doses are administered. In some embodiments, a total of 7 doses are administered. In some embodiments, a total of 8 doses are administered. In some embodiments, a total of 9 doses are administered. In some embodiments, a total of 10 doses are administered. In some embodiments, a total of more than 10 doses are administered.

Pharmaceutical compositions containing a CD70 binding antibody or antigen binding portion thereof or other CD70 binding agent or CD70 conjugate thereof can be administered in a unit dose. The term “unit dose” when used in reference to a pharmaceutical composition refers to physically discrete units suitable as unitary dosage for the subject, each unit containing a predetermined quantity of active material (e.g., a CD70 binding antibody or antigen binding portion thereof or other binding agent or conjugate thereof), calculated to produce the desired therapeutic effect in association with the required physiologically acceptable diluent, i.e., carrier, or vehicle.

In some embodiments, a CD70 binding antibody or an antigen binding portion thereof or other binding agent or conjugate thereof, or a pharmaceutical composition of any of these, is administered with an immunotherapy. As used herein, “immunotherapy” refers to therapeutic strategies designed to induce or augment the subject's own immune system to fight the cancer or malignancy. Examples of an immunotherapy include, but are not limited to, antibodies such as check point inhibitors.

In some embodiments, the immunotherapy involves administration of a checkpoint inhibitor. In some embodiments, an immune checkpoint inhibitor includes an agent that inhibits CTLA-4, PD-1, PD-L1, and the like. Suitable anti-CTLA-4 inhibitors include, for example, ipilimumab, tremelimumab, the antibodies disclosed in PCT Publication No. WO 2001/014424, the antibodies disclosed in PCT Publication No. WO 2004/035607, the antibodies disclosed in U.S. Publication No. 2005/0201994, and the antibodies disclosed in granted European Patent No. EP1212422B 1. Additional anti-CTLA-4 antibodies are described in U.S. Pat. Nos. 5,811,097, 5,855,887, 6,051,227, and 6,984,720; in PCT Publication Nos. WO 01/14424 and WO 00/37504; and in U.S. Publication Nos. 2002/0039581 and 2002/086014. Other anti-CTLA-4 antibodies that can be used in a method of the present invention include, for example, those disclosed in: WO 98/42752; U.S. Pat. Nos. 6,682,736 and 6,207,156; Hurwitz et al., Proc. Natl. Acad. Sci. USA, 95(17): 10067-10071 (1998); Camacho et al., J. Clin. Oncology, 22(145): Abstract No. 2505 (2004) (antibody CP-675206); Mokyr et al., Cancer Res, 58:5301-5304 (1998), U.S. Pat. Nos. 5,977,318, 6,682,736, 7,109,003, and 7,132,281.

Suitable anti-PD-1 inhibitors, include, for example, nivolumab, pembrolizumab, pidilizumab, MEDI0680, and combinations thereof. In other specific embodiments, anti-PD-L1 therapy agents include atezolizumab, BMS-936559, MEDI4736, MSB0010718C, and combinations thereof.

Suitable anti-PD-1 inhibitors include, for example, those described in Topalian, et al., Immune Checkpoint Blockade: A Common Denominator Approach to Cancer Therapy, Cancer Cell 27: 450-61 (Apr. 13, 2015), incorporated herein by reference in its entirety.

In some embodiments, the checkpoint inhibitor is Ipilimumab (Yervoy), Nivolumab (Opdivo), Pembrolizumab (Keytruda), Atezolizumab (Tecentriq), Avelumab (Bavencio), or Durvalumab (Imfinzi).

In some embodiments, provided is a method of improving treatment outcome in a subject receiving immunotherapy. The method generally includes administering an effective amount of an immunotherapy to the subject having cancer; and administering a therapeutically effective amount of a CD70 antibody, antigen binding portion, other binding agent or conjugate thereof or a pharmaceutical composition thereof to the subject, wherein the CD70 antibody, antigen binding portion, other binding agent or conjugate thereof specifically binds to CD70+ cancer cells; wherein the treatment outcome of the subject is improved, as compared to administration of the immunotherapy alone. In some embodiments, the CD70 antibody, antigen binding portion, other binding agent or conjugate thereof comprises any of the embodiments of CD70 antibodies, antigen binding portions, other binding agents or conjugates thereof as described herein.

In some embodiments, an improved treatment outcome is an objective response selected from stable disease, a partial response or a complete response as determined by standard medical criteria for the cancer being treated. In some embodiments, an improved treatment outcome is reduced tumor burden. In some embodiments, an improved treatment outcome is progression-free survival or disease-free survival.

### Treatment of Autoimmune Disease

In some embodiments, the CD70 antibodies or antigen binding portions thereof, other binding agents and conjugates as described herein can be used in a method(s) comprising administering a CD70 antibody or antigen-binding portion thereof or other binding agent or conjugate thereof as described herein to a subject in need thereof, such as a subject having an autoimmune disease.

In some embodiments, provided are methods of treating an autoimmune disease comprising administering a CD70 antibody or antigen-binding portion thereof or other binding agent or conjugate thereof comprising a heavy chain variable region (VH) and a light chain variable region (VL), the VH and VL regions having amino acid sequences set forth in the pairs of amino acid sequences selected from SEQ ID NO:3 and SEQ ID NO:4, respectively; SEQ ID NO:5 and SEQ ID NO:6, respectively; SEQ ID NO:7 and SEQ ID NO:8, respectively; SEQ ID NO:9 and SEQ ID NO:10, respectively; and SEQ ID NO:11 and SEQ ID NO:12; respectively. In some embodiments, provided are methods of treating an autoimmune disease comprising administering a CD70 antibody or antigen-binding portion thereof or other binding agent or conjugate thereof comprising a heavy chain variable region (VH) and a light chain variable region (VL), the VH and VL regions having amino acid sequences set forth in SEQ ID NO:3 and SEQ ID NO:4, respectively. In some embodiments, provided are methods of treating an autoimmune disease comprising administering a CD70 antibody or antigen-binding portion thereof or other binding agent or conjugate thereof comprising a heavy chain variable region (VH) and a light chain variable region (VL), the VH and VL regions having amino acid sequences set forth in SEQ ID NO:5 and SEQ ID NO:6, respectively. In some embodiments, provided are methods of treating an autoimmune disease comprising administering a CD70 antibody or antigen-binding portion thereof or other binding agent or conjugate thereof comprising a heavy chain variable region (VH) and a light chain variable region (VL), the VH and VL regions having amino acid sequences set forth in SEQ ID NO:7 and SEQ ID NO:8, respectively. In some embodiments, provided are methods of treating an autoimmune disease comprising administering a CD70 antibody or antigen-binding portion thereof or other binding agent or conjugate thereof comprising a heavy chain variable region (VH) and a light chain variable region (VL), the VH and VL regions having amino acid sequences set forth in SEQ ID NO:9 and SEQ ID NO:10, respectively. In some embodiments, provided are methods of treating an autoimmune disease comprising administering a CD70 antibody or antigen-binding portion thereof or other binding agent or conjugate thereof comprising a heavy chain variable region (VH) and a light chain variable region (VL), the VH and VL regions having amino acid sequences set forth in and SEQ ID NO:11 and SEQ ID NO:12; respectively.

In some embodiments, provided are methods of treating an autoimmune disease comprising administering a CD70 antibody or antigen-binding portion thereof or other binding agent or conjugate thereof comprising a heavy chain variable region (VH) and a light chain variable region (VL), the VH and VL regions having amino acid sequences set forth in the pairs of amino acid sequences selected from SEQ ID NO:3 and SEQ ID NO:4, respectively; SEQ ID NO:5 and SEQ ID NO:6, respectively; SEQ ID NO:7 and SEQ ID NO:8, respectively; SEQ ID NO:9 and SEQ ID NO:10, respectively; and SEQ ID NO:11 and SEQ ID NO:12; respectively; wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified. In some embodiments, provided are methods of treating an autoimmune disease comprising administering a CD70 antibody or antigen-binding portion thereof or other binding agent or conjugate thereof comprising a heavy chain variable region (VH) and a light chain variable region (VL), the VH and VL regions having amino acid sequences set forth in SEQ ID NO:3 and SEQ ID NO:4, respectively; wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified. In some embodiments, provided are methods of treating an autoimmune disease comprising administering a CD70 antibody or antigen-binding portion thereof or other binding agent or conjugate thereof comprising a heavy chain variable region (VH) and a light chain variable region (VL), the VH and VL regions having amino acid sequences set forth in SEQ ID NO:5 and SEQ ID NO:6, respectively; wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified. In some embodiments, provided are methods of treating an autoimmune disease comprising administering a CD70 antibody or antigen-binding portion thereof or other binding agent or conjugate thereof comprising a heavy chain variable region (VH) and a light chain variable region (VL), the VH and VL regions having amino acid sequences set forth in SEQ ID NO:7 and SEQ ID NO:8, respectively; wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified. In some embodiments, provided are methods of treating an autoimmune disease comprising administering a CD70 antibody or antigen-binding portion thereof or other binding agent or conjugate thereof comprising a heavy chain variable region (VH) and a light chain variable region (VL), the VH and VL regions having amino acid sequences set forth in SEQ ID NO:9 and SEQ ID NO:10, respectively; wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified. In some embodiments, provided are methods of treating an autoimmune disease comprising administering a CD70 antibody or antigen-binding portion thereof or other binding agent or conjugate thereof comprising a heavy chain variable region (VH) and a light chain variable region (VL), the VH and VL regions having amino acid sequences set forth in SEQ ID NO:11 and SEQ ID NO:12; respectively; wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified.

In some embodiments, provided are methods of treating an autoimmune disease comprising administering a CD70 antibody or antigen-binding portion thereof or other binding agent or conjugate thereof comprising a heavy chain variable region (VH) and a light chain variable region (VL), the VH and VL regions having the amino acid sequences set forth in the pairs of amino acid sequences selected from SEQ ID NO:3 and SEQ ID NO:4, respectively; SEQ ID NO:5 and SEQ ID NO:6, respectively; SEQ ID NO:7 and SEQ ID NO:8, respectively; SEQ ID NO:9 and SEQ ID NO:10, respectively; and SEQ ID NO:11 and SEQ ID NO:12; respectively; wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified. In some embodiments, provided are methods of treating an autoimmune disease comprising administering a CD70 antibody or antigen-binding portion thereof or other binding agent or conjugate thereof comprising a heavy chain variable region (VH) and a light chain variable region (VL), the VH and VL regions having the amino acid sequences set forth in SEQ ID NO:3 and SEQ ID NO:4, respectively; wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified. In some embodiments, provided are methods of treating an autoimmune disease comprising administering a CD70 antibody or antigen-binding portion thereof or other binding agent or conjugate thereof comprising a heavy chain variable region (VH) and a light chain variable region (VL), the VH and VL regions having the amino acid sequences set forth in SEQ ID NO:5 and SEQ ID NO:6, respectively; wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified. In some embodiments, provided are methods of treating an autoimmune disease comprising administering a CD70 antibody or antigen-binding portion thereof or other binding agent or conjugate thereof comprising a heavy chain variable region (VH) and a light chain variable region (VL), the VH and VL regions having the amino acid sequences set forth in SEQ ID NO:7 and SEQ ID NO:8, respectively; wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified. In some embodiments, provided are methods of treating an autoimmune disease comprising administering a CD70 antibody or antigen-binding portion thereof or other binding agent or conjugate thereof comprising a heavy chain variable region (VH) and a light chain variable region (VL), the VH and VL regions having the amino acid sequences set forth in SEQ ID NO:9 and SEQ ID NO:10, respectively; wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified. In some embodiments, provided are methods of treating an autoimmune disease comprising administering a CD70 antibody or antigen-binding portion thereof or other binding agent or conjugate thereof comprising a heavy chain variable region (VH) and a light chain variable region (VL), the VH and VL regions having the amino acid sequences set forth in SEQ ID NO: 11 and SEQ ID NO:12; respectively; wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified.

In some embodiments, provided are methods of treating an autoimmune disease comprising administering a CD70 antibody or antigen-binding portion thereof or other binding agent or conjugate thereof comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having the amino acids sequences set forth in the sets of amino acid sequences selected from (i) SEQ ID NO:21, SEQ ID NO:22, SEQ ID NO:13, SEQ ID NO:24, SEQ ID NO:25 and SEQ ID NO:26, respectively; (ii) SEQ ID NO:21, SEQ ID NO:22, SEQ ID NO:14, SEQ ID NO:24, SEQ ID NO:25 and SEQ ID NO:26, respectively; (iii) SEQ ID NO:21, SEQ ID NO:22, SEQ ID NO:15, SEQ ID NO:24, SEQ ID NO:25 and SEQ ID NO:26, respectively; (iv) SEQ ID NO:21, SEQ ID NO:22, SEQ ID NO:23, SEQ ID NO:24, SEQ ID NO:25 and SEQ ID NO:18, respectively; and (v) SEQ ID NO:16, SEQ ID NO:17, SEQ ID NO:23, SEQ ID NO:24, SEQ ID NO:25 and SEQ ID NO:26; respectively. In some embodiments, each VH and VL region comprises a humanized framework region. In some embodiments, each VH and VL region comprises a human framework region.

In some embodiments, provided are methods of treating an autoimmune disease comprising administering a CD70 antibody or antigen-binding portion thereof or other binding agent or conjugate thereof comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having the amino acids sequences set forth in SEQ ID NO:21, SEQ ID NO:22, SEQ ID NO:13, SEQ ID NO:24, SEQ ID NO:25 and SEQ ID NO:26, respectively. In some embodiments, each VH and VL region comprises a humanized framework region. In some embodiments, each VH and VL region comprises a human framework region.

In some embodiments, provided are methods of treating an autoimmune disease comprising administering a CD70 antibody or antigen-binding portion thereof or other binding agent or conjugate thereof comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having the amino acids sequences set forth in SEQ ID NO:21, SEQ ID NO:22, SEQ ID NO:14, SEQ ID NO:24, SEQ ID NO:25 and SEQ ID NO:26, respectively. In some embodiments, each VH and VL region comprises a humanized framework region. In some embodiments, each VH and VL region comprises a human framework region.

In some embodiments, provided are methods of treating an autoimmune disease comprising administering a CD70 antibody or antigen-binding portion thereof or other binding agent or conjugate thereof comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having the amino acids sequences set forth in SEQ ID NO:21, SEQ ID NO:22, SEQ ID NO:15, SEQ ID NO:24, SEQ ID NO:25 and SEQ ID NO:26, respectively. In some embodiments, each VH and VL region comprises a humanized framework region. In some embodiments, each VH and VL region comprises a human framework region.

In some embodiments, provided are methods of treating an autoimmune disease comprising administering a CD70 antibody or antigen-binding portion thereof or other binding agent or conjugate thereof comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having the amino acids sequences set forth in SEQ ID NO:21, SEQ ID NO:22, SEQ ID NO:23, SEQ ID NO:24, SEQ ID NO:25 and SEQ ID NO:18, respectively. In some embodiments, each VH and VL region comprises a humanized framework region. In some embodiments, each VH and VL region comprises a human framework region.

In some embodiments, provided are methods of treating an autoimmune disease comprising administering a CD70 antibody or antigen-binding portion thereof or other binding agent or conjugate thereof comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having the amino acids sequences set forth in SEQ ID NO:16, SEQ ID NO:17, SEQ ID NO:23, SEQ ID NO:24, SEQ ID NO:25 and SEQ ID NO:26; respectively. In some embodiments, each VH and VL region comprises a humanized framework region. In some embodiments, each VH and VL region comprises a human framework region.

In some embodiments, the subject is in need of treatment for an autoimmune disease. The methods described herein include administering a therapeutically effective amount of a CD70 binding antibody or antigen binding portion thereof or other binding agent or conjugate thereof to a subject having an autoimmune disease. As used herein, the phrase “therapeutically effective amount”, “effective amount” or “effective dose” refers to an amount of the CD70 antibody or antigen binding portion thereof or other binding agent or conjugate as described herein that provides a therapeutic benefit in the treatment of, management of or prevention of relapse of an autoimmune disease, e.g., an amount that provides a statistically significant decrease in at least one symptom, sign, or marker of an autoimmune disease. Determination of a therapeutically effective amount is well within the capability of those skilled in the art. Generally, a therapeutically effective amount can vary with the subject's history, age, condition, sex, as well as the severity and type of the medical condition in the subject, and administration of other pharmaceutically active agents.

The term “autoimmune disease” refers to an immunological disorder characterized by expression of CD70 by inappropriate activation of immune cells (e.g., lymphocytes or dendritic cells), that interferes with the normal functioning of the bodily organs and systems. Examples of autoimmune disease include, but are not limited to, rheumatoid arthritis, psoriatic arthritis, autoimmune demyelinative diseases (e.g., multiple sclerosis, allergic encephalomyelitis), endocrine ophthalmopathy, uveoretinitis, systemic lupus erythematosus, myasthenia gravis, Grave's disease, glomerulonephritis, autoimmune hepatological disorder, inflammatory bowel disease (e.g., Crohn's disease), anaphylaxis, allergic reaction, Sjogren's syndrome, type I diabetes mellitus, primary biliary cirrhosis, Wegener's granulomatosis, fibromyalgia, polymyositis, dermatomyositis, multiple endocrine failure, Schmidt's syndrome, autoimmune uveitis, Addison's disease, adrenalitis, thyroiditis, Hashimoto's thyroiditis, autoimmune thyroid disease, pernicious anemia, gastric atrophy, chronic hepatitis, lupoid hepatitis, atherosclerosis, subacute cutaneous lupus erythematosus, hypoparathyroidism, Dressler's syndrome, autoimmune thrombocytopenia, idiopathic thrombocytopenic purpura, hemolytic anemia, pemphigus vulgaris, pemphigus, dermatitis herpetiformis, alopecia areata, pemphigoid, scleroderma, progressive systemic sclerosis, CREST syndrome (calcinosis, Raynaud's phenomenon, esophageal dysmotility, sclerodactyl), and telangiectasia), male and female autoimmune infertility, ankylosing spondolytis, ulcerative colitis, mixed connective tissue disease, polyarteritis nodosa, systemic necrotizing vasculitis, atopic dermatitis, atopic rhinitis, Goodpasture's syndrome, Chagas' disease, sarcoidosis, rheumatic fever, asthma, recurrent abortion, anti-phospholipid syndrome, farmer's lung, erythema multiforme, post cardiotomy syndrome, Cushing's syndrome, autoimmune chronic active hepatitis, bird-fancier's lung, toxic epidermal necrolysis, Alport's syndrome, alveolitis, allergic alveolitis, fibrosing alveolitis, interstitial lung disease, erythema nodosum, pyoderma gangrenosum, transfusion reaction, Takayasu's arteritis, polymyalgia rheumatica, temporal arteritis, schistosomiasis, giant cell arteritis, ascariasis, aspergillosis, Samter's syndrome, eczema, lymphomatoid granulomatosis, Behcet's disease, Caplan's syndrome, Kawasaki's disease, dengue, encephalomyelitis, endocarditis, endomyocardial fibrosis, endophthalmitis, erythema elevatum et diutinum, psoriasis, erythroblastosis fetalis, eosinophilic faciitis, Shulman's syndrome, Felty's syndrome, filariasis, cyclitis, chronic cyclitis, heterochronic cyclitis, Fuch's cyclitis, IgA nephropathy, Henoch-Schonlein purpura, graft versus host disease, transplantation rejection, cardiomyopathy, Eaton-Lambert syndrome, relapsing polychondritis, cryoglobulinemia, Waldenstrom's macroglobulemia, Evan's syndrome, and autoimmune gonadal failure.

In some embodiments, the methods described herein encompass treatment of disorders of B lymphocytes (e.g., systemic lupus erythematosus, Goodpasture's syndrome, rheumatoid arthritis, and type I diabetes), Th1-lymphocytes (e.g., rheumatoid arthritis, multiple sclerosis, psoriasis, Sjorgren's syndrome, Hashimoto's thyroiditis, Grave's disease, primary biliary cirrhosis, Wegener's granulomatosis, tuberculosis, or graft versus host disease), or Th2-lymphocytes (e.g., atopic dermatitis, systemic lupus erythematosus, atopic asthma, rhinoconjunctivitis, allergic rhinitis, Omenn's syndrome, systemic sclerosis, or chronic graft versus host disease). Generally, disorders involving dendritic cells involve disorders of Th1-lymphocytes or Th2-lymphocytes.

In some embodiments, the immunological disorder is a T cell-mediated immunological disorder, such as a T cell disorder in which activated T cells associated with the disorder express CD70. CD70 antibodies, antigen binding portions, other binding agents and conjugates can be administered to deplete such CD70-expressing activated T cells. In a specific embodiment, administration of CD70 antibodies antigen binding portions, other binding agents and conjugates can deplete CD70-expressing activated T cells, while resting T cells are not substantially depleted by the anti-CD70 antigen binding portions, other binding agents and conjugates. In this context, “not substantially depleted” means that less than about 60%, or less than about 70% or less than about 80% of resting T cells are not depleted.

As used herein, a “subject” refers to a human or animal. Usually the animal is a vertebrate such as a primate, rodent, domestic animal or game animal. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In certain embodiments, the subject is a mammal, e.g., a primate, e.g., a human. The terms, “patient”, “individual” and “subject” are used interchangeably herein.

Preferably, the subject is a mammal. The mammal can be a human, non-human primate, mouse, rat, dog, cat, horse, or cow, but are not limited to these examples. Mammals other than humans can be advantageously used, for example, as subjects that represent animal models of, for example, various autoimmune diseases. In addition, the methods described herein can be used to treat domesticated animals and/or pets. A subject can be male or female. In certain embodiments, the subject is a human.

In some embodiments, a subject can be one who has been previously diagnosed with or identified as suffering from an autoimmune disease and in need of treatment, but need not have already undergone treatment for the autoimmune disease. In some embodiments, a subject can also be one who has not been previously diagnosed as having an autoimmune disease in need of treatment. In some embodiments, a subject can be one who exhibits one or more risk factors for a condition or one or more complications related to an autoimmune disease or a subject who does not exhibit risk factors. A “subject in need” of treatment for an autoimmune disease particular can be a subject having that condition or diagnosed as having that condition. In other embodiments, a subject “at risk of developing” a condition refers to a subject diagnosed as being at risk for developing the condition or at risk for having the condition again (e.g., an autoimmune disease).

As used herein, the terms “treat,” “treatment,” “treating,” or “amelioration” when used in reference to a disease, disorder or medical condition, refer to therapeutic treatments for a condition, wherein the object is to reverse, alleviate, ameliorate, inhibit, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a condition is reduced or halted. That is, “treatment” includes not just the improvement of symptoms or markers, but also a cessation or at least slowing of progress or worsening of symptoms that would be expected in the absence of treatment. Beneficial or desired clinical results include, but are not limited to, reduction in CD70+ autoimmune cells in the subject, alleviation of one or more symptom(s), diminishment of extent of the deficit, stabilized (i.e., not worsening) state of an autoimmune disease, delay or slowing of progression of an autoimmune disease, and an increased lifespan as compared to that expected in the absence of treatment. As used herein, the term “administering,” refers to providing a CD70 binding antibody or antigen-binding portion thereof or other binding agent or conjugate as described herein or a nucleic acid encoding the CD70 antibody or antigen-binding portion thereof or other binding agent as described herein into a subject by a method or route which results in binding to the CD70 binding antibody or antigen binding portion thereof or other binding agent or conjugate to CD70+ autoimmune cells. Similarly, a pharmaceutical composition comprising a CD70 binding antibody or antigen-binding portion thereof or other binding agent or conjugate as described herein or a nucleic acid encoding the CD70 antibody or antigen-binding portion thereof or other binding agent as described herein disclosed herein can be administered by any appropriate route which results in an effective treatment in the subject.

The dosage ranges for a CD70 binding antibody or antigen binding portion thereof or binding agent or conjugate depend upon the potency, and encompass amounts large enough to produce the desired effect e.g., slowing of progression of an autoimmune disease or a reduction of symptoms. The dosage should not be so large as to cause unacceptable adverse side effects. Generally, the dosage will vary with the age, condition, and sex of the subject and can be determined by one of skill in the art. The dosage can also be adjusted by the individual physician in the event of any complication. In some embodiments, the dosage ranges from 0.1 mg/kg body weight to 10 mg/kg body weight. In some embodiments, the dosage ranges from 0.5 mg/kg body weight to 15 mg/kg body weight. In some embodiments, the dose range is from 0.5 mg/kg body weight to 5 mg/kg body weight. Alternatively, the dose range can be titrated to maintain serum levels between 1 ug/mL and 1000 ug/mL. For systemic administration, subjects can be administered a therapeutic amount, such as, e.g. 0.1 mg/kg, 0.5 mg/kg, 1.0 mg/kg, 2.0 mg/kg, 2.5 mg/kg, 5 mg/kg, 10 mg/kg, 12 mg/kg or more.

Administration of the doses recited above can be repeated. In a preferred embodiment, the doses recited above are administered weekly, biweekly, every three weeks or monthly for several weeks or months. The duration of treatment depends upon the subject's clinical progress and responsiveness to treatment.

In some embodiments, a dose can be from about 0.1 mg/kg to about 100 mg/kg. In some embodiments, a dose can be from about 0.1 mg/kg to about 25 mg/kg. In some embodiments, a dose can be from about 0.1 mg/kg to about 20 mg/kg. In some embodiments, a dose can be from about 0.1 mg/kg to about 15 mg/kg. In some embodiments, a dose can be from about 0.1 mg/kg to about 12 mg/kg. In some embodiments, a dose can be from about 1 mg/kg to about 100 mg/kg. In some embodiments, a dose can be from about 1 mg/kg to about 25 mg/kg. In some embodiments, a dose can be from about 1 mg/kg to about 20 mg/kg. In some embodiments, a dose can be from about 1 mg/kg to about 15 mg/kg. In some embodiments, a dose can be from about 1 mg/kg to about 12 mg/kg. In some embodiments, a dose can be from about 1 mg/kg to about 10 mg/kg.

In some embodiments, a dose can be administered intravenously. In some embodiments, an intravenous administration can be an infusion occurring over a period of from about 10 minutes to about 4 hours. In some embodiments, an intravenous administration can be an infusion occurring over a period of from about 30 minutes to about 90 minutes.

In some embodiments, a dose can be administered weekly. In some embodiments, a dose can be administered bi-weekly. In some embodiments, a dose can be administered about every 2 weeks. In some embodiments, a dose can be administered about every 3 weeks. In some embodiments, a dose can be administered every four weeks.

In some embodiments, a total of from about 2 to about 10 doses are administered to a subject. In some embodiments, a total of 4 doses are administered. In some embodiments, a total of 5 doses are administered. In some embodiments, a total of 6 doses are administered. In some embodiments, a total of 7 doses are administered. In some embodiments, a total of 8 doses are administered. In some embodiments, a total of 9 doses are administered. In some embodiments, a total of 10 doses are administered. In some embodiments, a total of more than 10 doses are administered.

Pharmaceutical compositions containing a CD70 binding antibody or antigen binding portion thereof or other CD70 binding agent or CD70 conjugate thereof can be administered in a unit dose. The term “unit dose” when used in reference to a pharmaceutical composition refers to physically discrete units suitable as unitary dosage for the subject, each unit containing a predetermined quantity of active material (e.g., a CD70 binding antibody or antigen binding portion thereof or other binding agent or conjugate thereof), calculated to produce the desired therapeutic effect in association with the required physiologically acceptable diluent, i.e., carrier, or vehicle.

In some embodiments, a CD70 binding antibody or an antigen binding portion thereof or other binding agent or conjugate thereof, or a pharmaceutical composition of any of these, is administered with an immunosuppressive therapy. In some embodiments, provided is a method of improving treatment outcome in a subject receiving immunosuppressive therapy. The method generally includes administering an effective amount of an immunosuppressive therapy to the subject having an autoimmune disorder; and administering a therapeutically effective amount of a CD70 antibody, antigen binding portion, other binding agent or conjugate thereof or a pharmaceutical composition thereof to the subject, wherein the CD70 antibody, antigen binding portion, other binding agent or conjugate thereof specifically binds to CD70+ autoimmune cells; wherein the treatment outcome of the subject is improved, as compared to administration of the immunotherapy alone. In some embodiments, the CD70 antibody, antigen binding portion, other binding agent or conjugate thereof comprises any of the embodiments of CD70 antibodies, antigen binding portions, other binding agents or conjugates thereof as described herein. In some embodiments, an improved treatment outcome is a decrease in disease progression, an alleviation of one or more symptoms, or the like.

The present invention is further illustrated by the following embodiments which should not be construed as limiting.

1. A binding agent comprising:
a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having amino acids sequences selected from the sets of amino acid sequences set forth in the group consisting of:
   SEQ ID NO:21, SEQ ID NO:22, SEQ ID NO:13, SEQ ID NO:24, SEQ ID NO:25 and
   SEQ ID NO:26, respectively;
   SEQ ID NO:21, SEQ ID NO:22, SEQ ID NO:14, SEQ ID NO:24, SEQ ID NO:25 and
   SEQ ID NO:26, respectively;
   SEQ ID NO:21, SEQ ID NO:22, SEQ ID NO:15, SEQ ID NO:24, SEQ ID NO:25 and
   SEQ ID NO:26, respectively;
   SEQ ID NO:21, SEQ ID NO:22, SEQ ID NO:23, SEQ ID NO:24, SEQ ID NO:25 and
   SEQ ID NO:18, respectively; and
   SEQ ID NO:16, SEQ ID NO:17, SEQ ID NO:23, SEQ ID NO:24, SEQ ID NO:25 and
   SEQ ID NO:26; respectively.
      2. The binding agent of embodiment 1, wherein the VH and VL regions have amino acid sequences that are selected from the pairs of amino acid sequences set forth in the group consisting of:
SEQ ID NO:3 and SEQ ID NO:4;
SEQ ID NO:5 and SEQ ID NO:6;
SEQ ID NO:7 and SEQ ID NO:8;
SEQ ID NO:9 and SEQ ID NO:10; and
SEQ ID NO:11 and SEQ ID NO:12; respectively.
   3. The binding agent of embodiment 1, wherein the VH and VL regions have amino acid sequences that are selected from the pairs of amino acid sequences set forth in the group consisting of:
SEQ ID NO:3 and SEQ ID NO:4;
SEQ ID NO:5 and SEQ ID NO:6;
SEQ ID NO:7 and SEQ ID NO:8;
SEQ ID NO:9 and SEQ ID NO:10; and
SEQ ID NO:11 and SEQ ID NO:12; respectively,
wherein the heavy and light chain framework regions are optionally modified with from 1 to 8 amino acid substitutions, deletions or insertions in the framework regions.
   4. The binding agent of any of the preceding embodiments, wherein HCDR1, HCDR2 and HCDR3 and LCDR1, LCDR2 and LCDR3 have the amino acid sequences set forth in SEQ ID NO:21, SEQ ID NO:22, and SEQ ID NO:15, and SEQ ID NO:24, SEQ ID NO:25 and SEQ ID NO:26, respectively.
   5. The binding agent of embodiment 1, wherein the framework regions are human framework regions.
   6. The binding agent of any of embodiments 1 to 5, wherein the binding agent is an antibody or an antigen-binding portion thereof.
   7. The binding agent of any of the preceding embodiments, wherein the binding agent is a monoclonal antibody, a Fab, a Fab′, an F(ab′), an Fv, a disulfide linked Fc, a scFv, a single domain antibody, a diabody, a bi-specific antibody, or a multi-specific antibody.
   8. The binding agent of any of the preceding embodiments, wherein the heavy chain variable region further comprises a heavy chain constant region.
   9. The binding agent of embodiment 8, wherein heavy chain constant region is of the IgG isotype.
   10. The binding agent of embodiment 9, wherein the heavy chain constant region is an IgG1 constant region.
   11. The binding agent of embodiment 8, wherein the heavy chain constant region is an IgG4 constant region.
   12. The binding agent of embodiment 10, wherein the IgG1 constant region has the amino acid sequence set forth in SEQ ID NO:28.
   13. The binding agent of any of the preceding embodiments, wherein the light chain variable region further comprises a light chain constant region.
   14. The binding agent of embodiment 13, wherein the light chain constant region is of the kappa isotype.
   15. The binding agent of embodiment 14, wherein the light chain constant region has the amino acid sequence set forth in SEQ ID NO:29.
   16. The binding agent of any of embodiments 8 to 18, wherein the heavy chain constant region further comprises at least amino acid modification that decreases binding affinity to human FcgammaRIII.
   17. The binding agent of any of the preceding embodiments, wherein the binding agent is mono-specific.
   18. The binding agent of any of embodiments 1 to 17, wherein the binding agent is bivalent.
   19. The binding agent of any of embodiments 1 to 17, wherein the binding agent is bispecific.
   20. A pharmaceutical composition comprising the binding agent of any of embodiments 1 to 19 and a pharmaceutically acceptable carrier.
   21. A nucleic acid encoding the binding agent of any of embodiments 1 to 19.
   22. A vector comprising the nucleic acid of embodiment 21.
   23. A cell line comprising the vector of embodiment 22.
   24. A conjugate comprising:
the binding agent of any of embodiments 1 to 19,
at least one linker attached to the binding agent; and
at least one drug attached to each linker.
   25. The conjugate of embodiment 24, wherein each drug is selected from a cytotoxic agent, an immunomodulatory agents, a nucleic acid, a growth inhibitory agent, a PROTAC, a toxin and a radioactive isotopes.
   26. The conjugate of any of embodiments 24 to 25, wherein each linker is attached to the binding agent via an interchain disulfide residue, a lysine residue, an engineered cysteine residue, a glycan, a modified glycan, an N-terminal residue of the binding agent or a polyhistidine residue attached to the binding agent.
   27. The conjugate of any of embodiments 24 to 26, wherein the average drug loading of the conjugate is from about 1 to about 8, about 2, about 4, about 6, about 8, about 10, about 12, about 14, about 16, about 3 to about 5, about 6 to about 8, or about 8 to about 16.
   28. The conjugate of any of embodiments 24 to 27, wherein the drug is a cytotoxic agent.
   29. The conjugate of embodiment 28, wherein the cytotoxic agent is selected from the group consisting of an auristatin, a maytansinoid, a camptothecin, a duocarmycin, or a calicheamicin.
   30. The conjugate of embodiment 29, wherein the cytotoxic agent is an auristatin.
   31. The conjugate of embodiment 30, wherein the cytotoxic agent is MMAE or MMAF.
   32. The conjugate of embodiment 29, wherein the cytotoxic agent is a camptothecin.
   33. The conjugate of embodiment 32, wherein the cytotoxic agent is exatecan.
   34. The conjugate of embodiment 32, wherein the cytotoxic agent is SN-38.
   35. The conjugate of embodiment 29, wherein the cytotoxic agent is a calicheamicin.
   36. The conjugate of embodiment 29, wherein the cytotoxic agent is a maytansinoid.
   37. The conjugate of embodiment 36, wherein the maytansinoid is maytansine, maytansinol or a maytansine analog in DM1, DM3 and DM4, and ansamatocin-2.
   38. The conjugate of any of embodiments 24 to 37, wherein the linker is a cleavable linker.
   39. The conjugate of embodiment 38, wherein the linker comprises mc-VC-PAB, CL2, CL2A or (Succinimid-3-yl-N)—(CH2)n-C(═O)-Gly-Gly-Phe-Gly-NH—CH2-O—CH2-(C═O)—, wherein n=1 to 5.
   40. The conjugate of embodiment 39, wherein the linker comprises mc-VC-PAB.
   41. The conjugate of embodiment 39, wherein the linker comprises CL2A.
   42. The conjugate of embodiment 39, wherein the linker comprises CL2.
   43. The conjugate of embodiment 39, wherein the linker comprises (Succinimid-3-yl-N)—(CH2)n-C(═O)-Gly-Gly-Phe-Gly-NH—CH2-O—CH2-(C═O)—.
   44. The conjugate of embodiment 43, wherein the linker is attached to at least one molecule of exatecan.
   45. The conjugate of any of embodiments 24 to 27, wherein the drug is an immune modulatory agent.
   46. The conjugate of embodiment 45, wherein the immune modulatory agent is selected from the group consisting of a TRL7 agonist, a TLR8 agonist, a STING agonist, or a RIG-1 agonist.
   47. The conjugate of embodiment 46, wherein the immune modulatory agent is an TLR7 agonist.
   48. The conjugate of embodiment 46, wherein the TLR7 agonist is an imidazoquinoline, an imidazoquinoline amine, a thiazoquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine, heteroarothiadiazide-2,2-dioxide, a benzonaphthyridine, a guanosine analog, an adenosine analog, a thymidine homopolymer, ssRNA, CpG-A, PolyG10, and PolyG3.
   49. The conjugate of embodiment 45, wherein the immune modulatory agent is a TLR8 agonist.
   50. The conjugate of embodiment 49, wherein the TLR8 agonist is selected from an imidazoquinoline, a thiazoloquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine or a ssRNA.
   51. The conjugate of embodiment 45, wherein the immune modulatory agent is a STING agonist.
   52. The conjugate of embodiment 45, wherein the immune modulatory agent is a RIG-1 agonist.
   53. The conjugate of embodiment 52, wherein the RIG-1 agonist is selected from KIN1148, SB-9200, KIN700, KIN600, KIN500, KIN100, KIN101, KIN400 and KIN2000.
   54. The conjugate of any of embodiments 45 to 53, wherein the linker is selected from the group consisting of mc-VC-PAB, CL2, CL2A and (Succinimid-3-yl-N)—(CH2)n-C(═O)-Gly-Gly-Phe-Gly-NH—CH2-O—CH2-(C═O)—, wherein n=1 to 5.
   55. A pharmaceutical composition comprising the conjugate of any of embodiments 24 to 54 and a pharmaceutically acceptable carrier.
   56. A method of treating a CD70+ cancer, comprising administering to a subject in need thereof a therapeutically effective amount of the binding agent of any of embodiments 1 to 19, the conjugate of any of embodiments 24 to 54 or the pharmaceutical composition of embodiments 20 or 55.
   57. The method of embodiment 56, wherein the CD70+ cancer is a solid tumor or a hematologic malignancy.
   58. The method of embodiment 57, wherein the CD70+ cancer is selected from hepatocellular cancer, colorectal cancer, pancreatic cancer, ovarian cancer, indolent Non-Hodgkin's Lymphoma, Non-Hodgkin's Lymphoma, cancers of the B-cell lineage, multiple myeloma, renal cell cancers, nasopharyngeal cancers, thymic cancers and gliomas.
   59. The method of embodiment 57, wherein the CD70 cancer is a solid tumor.
   60. The method of any of embodiments 56 to 59, further comprising administering an immunotherapy to the subject.
   61. The method of embodiment 60, wherein the immunotherapy comprises a checkpoint inhibitor.
   62. The method of embodiment 61, wherein the checkpoint inhibitor is selected from an antibody that specifically binds to human PD-1, human PD-L1, or human CTLA4.
   63. The method of embodiment 62, wherein the checkpoint inhibitor is pembrolizumab, nivolumab, cemiplimab or ipilimumab.
   64. The method of any of embodiments 56 to 63, further comprising administering chemotherapy to the subject.
   65. The method of any of embodiments 56 to 64, comprising administering the conjugate of embodiments 25 to 53 or the pharmaceutical composition of claim 55.
   66. The method of any of embodiments 56 to 65, wherein the binding agent, conjugate or pharmaceutical composition is administered intravenously.
   67. The method of embodiments 66, wherein the binding agent, conjugate or pharmaceutical composition is administered in a dose of about 0.1 mg/kg to about 12 mg/kg.
   68. The method of any of embodiments 56 to 67, wherein a treatment outcome of the subject is improved.
   69. The method of embodiment 68, wherein the improved treatment outcome is an objective response selected from stable disease, a partial response or a complete response.
   70. The method of embodiment 68, wherein the improved treatment outcome is reduced tumor burden.
   71. The method of embodiment 68, wherein the improved treatment outcome is progression-free survival or disease-free survival.
   72. Use of the binding agent of any of embodiments 1 to 19 or the pharmaceutical composition of embodiment 20 for the treatment of CD70+ cancer in a subject.
   73. Use of the conjugate of any of embodiments 24 to 54 or the pharmaceutical composition of embodiment 55 for the treatment of CD70+ cancer in a subject.
   74. A method of treating an autoimmune disease, comprising administering to a subject in need thereof a therapeutically effective amount of the binding agent of any of embodiments 1 to 19, the conjugate of any of embodiments 24 to 54 or the pharmaceutical composition of embodiments 20 or 55.
   75. The method of embodiment 74, wherein the autoimmune disease is rheumatoid arthritis, multiple sclerosis, or systemic lupus erythematosus.
   76. The method of any of embodiments 74 to 75, further comprising administering an immunosuppressive therapy to the subject.
   77. The method of any of embodiments 74 to 76, comprising administering the conjugate of embodiments 24 to 54 or the pharmaceutical composition of claim 55.
   78. The method of any of embodiments 74 to 77, wherein the binding agent, conjugate or pharmaceutical composition is administered intravenously.
   79. The method of embodiments 78, wherein the binding agent, conjugate or pharmaceutical composition is administered in a dose of about 0.1 mg/kg to about 12 mg/kg.
   80. The method of any of embodiments 74 to 79, wherein a treatment outcome of the subject is improved.
   81. The method of embodiment 80, wherein the improved treatment outcome is a reduction in disease progression or alleviation of disease severity.
   82. Use of the binding agent of any of embodiments 1 to 19 or the pharmaceutical composition of embodiment 20 for the treatment of an autoimmune disease in a subject.
   83. Use of the conjugate of any of embodiments 24 to 54 or the pharmaceutical composition of embodiment 55 for the treatment of an autoimmune disease in a subject.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

### EXAMPLES

### Example 1: Generation of Human Antibodies Against Human CD70

Anti-CD70 antibodies with higher affinity and better characteristics were generated by random mutation of the CDR regions of the antibody heavy and light chain of parent antibody 69A7 (see U.S. Pat. No. 8,124,738B2). The heavy and light variable region amino acid sequences of 69A7 are set forth in SEQ ID NOs: 1 and SEQ 2, respectively. The HCDR and LCDR amino acid sequences are set forth in SEQ ID NOs: 21 to 26.

### CDR Scan Library Construction

Random mutations were introduced into each of the 6 CDRs of parent antibody 69A7 (3 HCDR+3 LCDR) (Chothia numbering convention) by PCR based mutagenesis with degenerative primers. The spliced PCR products were used for the library construction following a standard protocol.

The CDR library size was about 2 billion (2×10⁹) based on serial-dilution titration. Random colonies were picked for sequencing. The alignment of some random sequences from the un-selected library results showed that the random mutation library had good sequence diversity (data not shown). A phagemid library was rescued and used in the following panning procedure.

### Panning and Screening

The following standard protocol was followed for library panning. Immune tubes were coated with 0.5 ml of CD70 antigen at indicated concentration (see panning summary, Table 1), and placed in a refrigerator overnight. The tube was washed once with PBS, blocked with 1% BSA/PBS and placed at RT (room temperature) for 1 hour. The tube was incubated with the library phage sample at indicated amount (CFU, see the panning summary, Table 1) at RT for 1 hour. The tube was washed for 10 times with PBST buffer. To elute the bound phage, 0.5 ml of 100 mM TEA (triethylamine) was added and incubated at RT for 2 mins. The eluate was then transferred to a new tube and neutralized immediately by adding 0.25 ml of 1.0M Tris-HCL, pH 8.0, and mixing. The eluant (0.75 ml) was added into 10 ml of exponentially growing E. coli TG1 (OD600˜0.5), mixed well and incubated without shaking at 37° C. (water bath) for 30 min. 10-fold dilutions of the culture were made in 2×TY media and 10 μL of each dilution was plated on TYE/amp/glu plates, incubated at 30° C. overnight. The next day, the colony number for each dilution was counted, and the CFU (colony form unit) for the panning output was calculated. The remaining culture was centrifuged at 2,800 g for 15 mins, resuspend in 0.5 ml of 2×TY media, plated on two 150 mm TYE/amp/glu plates, and incubated at 30° C. overnight. The next day, 3 ml of 2×TY/amp/glu media was added to each plate and the bacteria were scraped from the plate with a cell spreader. Glycerol stocks was made by mixing 1.5 ml of bacteria and 0.5 ml of 80% glycerol, placed the stock at −80° C.

To prepare phage particles for the next round of selection, the glycerol stocks were inoculated into 40 ml of 2×TY/amp/glu media, starting at OD600-0.01-0.05. The culture was grown at 37° C. with shaking (300 rpm) until the OD600 reached 0.4-0.6. The culture was infected by adding helper phage CM13 to the culture at a helper phage:bacteria ratio of 5-10:1. The bacterial culture was incubated at 37° C. for 30 minutes standing in a water bath with occasional mixing followed by shaking at 37° C. for 30 minutes. The bacterial culture was centrifuged at 3000 rpm for 20 minutes, and the supernatant was removed. The pellet was resuspended in 100 mL of 2×TY/amp/kan and grown with shaking at 30° C. overnight. The culture was then harvested by centrifuging at 6,000 g for 30 mins. The phage particles were precipitated by adding 1/5 volume of PEG solution into the supernatant followed by 1 h incubation on ice, and then centrifuging at 4,000 g for 20 mins at 4° C. The supernatant was thoroughly removed. The phage pellet was resuspended in 1-2 ml of cold PBS. Any residual bacteria were removed by micro-centrifugation at top speed for 5 mins at 4° C. The prepared phage were either used immediately for selection or stored at −80° C. in aliquots with 10% glycerol. The titer of the phage preparation was determined by infecting 100 μL of exponentially growing E. coli TG1 with 10-fold dilution of the phage solution (in 2×TY, down to 1011). The selection step was repeated, starting with step 1, for a total round of 3 rounds.

As discussed above, 3 rounds of panning were performed. The concentrations of the washing buffer PBS-Tween20 in the 2^{nd} and 3rd rounds was gradually increased with 0.2%, 0.3%, respectively, and the coating antigen in the 2^{nd} and 3rd rounds was gradually decreased to 4 μg and 2 ug, respectively. After 3 rounds of screening, the target positive enrichment rate reached 6.9×10⁵ (690 thousand) with a significant difference from the blank control, as shown in Table 1.

### ELISA Assays on Purified Phage Samples.

A sterile 96-well round-bottom microtiter plate was filled with 100 μl/well of 2×YT-2% Glucose. Single TG1 colonies were picked up from selection plates of the 3^{rd} (last) enriched round using sterile pipette tips and used to inoculate one well/colony. The plate was sealed with a breathable membrane and incubated at 30° C. overnight while shaking. This plate was designated the master plate. On the next day, an aliquot of the cultures was transferred to a new deep-well induction plate containing 400 μl/well 2×YT-0.1% Glucose. About 10 μl/well from the master plate was pipetted using a multichannel pipette to the new induction plate. The induction plate was incubated for about 2-4 hrs in the phage orbital shaker until the bacteria reached log-phase (37° C., 200 rpm). IPTG was added to each well at a final concentration of 0.2 mM and the plate was cultured overnight at 30° C. with shaking at 200 rpm. The next day, the induction plate was spun at 3,500 rpm for 10 min. The supernatant was used in the following scFv ELISA. (The plates were optionally placed at 4° C. for temporary storage; the supernatant could be used within 2 weeks.)

The binding of the phage to CD70 antigen was tested in a phage ELISA. Briefly, the antigen CD70 (ACRO, CDL-H5246) was diluted to 5 μg/ml and coated onto a microtiter plate overnight using 100 μL/well. The next day the plate was washed 2× with PBST (0.1% Tween20 in PBS) using 200 μL/well. The blocking buffer (2% milk in PBST) was added using 200 μL/well. The plate was placed at RT for 1 hr. The plate was washed two times with PBST. IPTG-induced culture supernatants were added to each well using 50 μL/well and placed at RT for 1 hr. The plate was washed 3-4 times with PBST. Anti-human-HRP was diluted at 1:5,000 into PBST using 50 μL/well. The plate was incubated at RT for 20 min. The plate was then washed again for 5 times with PBST. Fresh developing solution (10 ml of Developing buffer, 13.3 μL Amplex Red (5 mg/ml in DMSO), 3.3 μL H₂O₂) was prepared, added using 50 μL/well, and the color was developed at RT for 1-60 min. The plate was read at Ex=530 nm, Em=590 nm and cutoff=570 nm.

TABLE 1
Summary of Panning Procedure

| | Round 1 | Round 2 | Round 3 |
|---|---|---|---|
| Input | 7.6 × 10¹¹ | 9.6 × 10¹⁰ | 4.8 × 10¹⁰ |
| (AMX scFv) | | | |
| Output | 1.8 × 10⁶ | 2.6 × 10⁶ | 7.0 × 10⁴ |
| Enrichment | 4.2 × 10⁵ | 3.7 × 10⁴ | 6.9 × 10⁵ |
| CD70 antigen | 6 ug | 2 ug | 1 ug |

Using the scFv Elisa procedure described above, two 96-well plates of single colonies were picked, cultured, and induced for scFv expression. The scFv supernatant was used in the screening assay.

20 clones with positive signals were sequenced. The sequences of 5 selected clones are shown in Table 2. The HCDR and LCDR in each variable region are marked in bold. These clones have at least two more amino acid substitutions in HCDR3/LCDR3 (SEQ ID NOs: 13 to SEQ18) for each candidate antibody compared with the parent antibody 69A7. The 5 unique sequences, 2A4, 1 H8, 2E7, 2D2 and 1A4, were picked for further analysis. One new VH/VL combination was prepared; the VH/VL pair of 2A4P0L1 was derived from the VH of 2A4 and the VL of 2D2.

TABLE 2
Variable region sequence of anti-CD70 antibodies

| Clone Seq. | VH | VL |
|---|---|---|
| 2A4 | QVQLQESGPGLVKPSETLSLTCTVSGGSVSSDYYYWSWI | EIVLTQSPATLSLSPGERATLSCRASQSVSSYLAWYQQKPG |
| | RQPPGKGLEWLGYIYYSGSTNYNPSLKSRVTISVDTSKN | QAPRLLIFDASNRATGIPARFSGSGSGTDFTLTISSLEPEDFA |
| | QFSLKLRSVITADTAVYYCARGDGDYGGNVFPYWGQG | VYYCQQRSNWPLTFGGGTKVEIK (SEQ 4) |
| | TLVTVSS (SEQ 3) | |
| 1H8 | QVQLQESGPGLVKPSETLSLTCTVSGGSVSSDYYYWSWI | EIVLTQSPATLSLSPGERATLSCRASQSVSSYLAWYQQKPG |
| | RQPPGKGLEWLGYIYYSGSTNYNPSLKSRVTISVDTSKN | QAPRLLIFDASNRATGIPARFSGSGSGTDFTLTISSLEPEDFA |
| | QFSLKLRSVTTADTAVYYCARGDGDFMGVCFDYWGQG | VYYCQQRSNWPLTFGGGTKVEIK (SEQ 6) |
| | TLVTVSS (SEQ 5) | |
| 2E7 | QVQLQESGPGLVKPSETLSLTCTVSGGSVSSDYYYWSWI | EIVLTQSPATLSLSPGERATLSCRASQSVSSYLAWYQQKPG |
| | RQPPGKGLEWLGYIYYSGSTNYNPSLKSRVTISVDTSKN | QAPRLLIFDASNRATGIPARFSGSGSGTDFTLTISSLEPEDFA |
| | QFSLKLRSVTTADTAVYYCARGDGDFLGVCFDYWGQG | VYYCQQRSNWPLTFGGGTKVEIK (SEQ 8) |
| | TLVTVSS (SEQ 7) | |
| 2D2 | QVQLQESGPGLVKPSETLSLTCTVSGGSVSSDYYYWSWI | EIVLTQSPATLSLSPGERATLSCRASQSVSSYLAWYQQKPG |
| | RQPPGKGLEWLGYIYYSGSTNYNPSLKSRVTISVDTSKN | QAPRLLIFDASNRATGIPARFSGSGSGTDFTLTISSLEPEDFA |
| | QFSLKLRSVTTADTAVYYCARGDGDYGGNCFDYWGQG | VYYCQQRLKFPLTFGGGTKVEIK (SEQ 10) |
| | TLVTVSS (SEQ 9) | |
| 1A4 | QVQLQESGPGLVKPSETLSLTCTVSGGSVYSGYYYWSWI | EIVLTQSPATLSLSPGERATLSCRASQSVSSYLAWYQQKPG |
| | RQPPGKGLEWLGYFSLSGSTNYNPSLKSRVTISVDTSKN | QAPRLLIFDASNRATGIPARFSGSGSGTDFTLTISSLEPEDFA |
| | QFSLKLRSVTTADTAVYYCARGDGDYGGNCFDYWGQG | VYYCQQRSNWPLTFGGGTKVEIK (SEQ 12) |
| | TLVTVSS (SEQ 11) | |

### Example 2: scFV Lead Characterization

To rank the leads from Example 1 by binding affinity, the scFv expression levels were measured using ProbeLife, and based on the scFv concentrations, the specific binding was titrated by ELISA for the 5 lead candidates, 2A4, 1H8, 2E7, 2D2 and 1A4.

ScFv Quantification was performed as follows: The expression level in the culture supernatant was measured on a Gator system (ProbeLife). After pre-wetting the anti-His sensors in Q Buffer (Probe Life), the sensor was dipped into the scFv supernatant wells for 2 mins. Based on the standard curve for the anti-His sensor, the expression titers were calculated by the system. The ELISA assay used is described above.

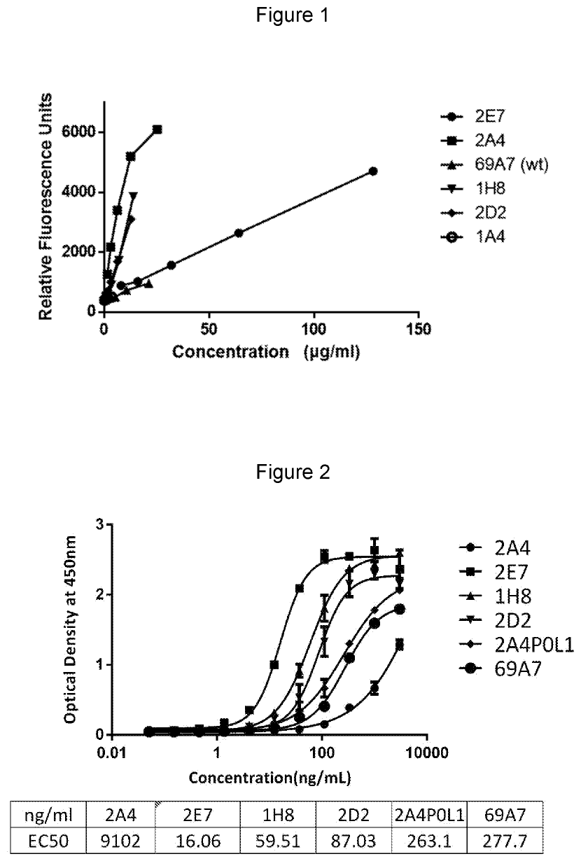

The result are shown in Table 3 and FIG. 1. Most of the lead antibodies (except for 2E7) had a similar or lower expression level than the parent antibody, 69A7. But all leads had significantly improved binding compared to the parental 69A7 scFv clone.

TABLE 3
Expression of the
leads (scFv) ranking

| Sample | Calculated C |
|---|---|
| (scFv) | (μg/mL) |
| 69A7 | 21.3 |
| (wild type) | |
| 1A4 | 3.51 |
| 1H8 | 14 |
| 2A4 | 25.3 |
| 2E7 | 128 |
| 2D2 | 12.8 |

### Example 3: Characterization of Anti-Human CD70 Antibodies

To further rank the leads in binding affinity and internalization, the scFv were converted into full IgG antibodies and the full IgG expression levels were measured. Based on the antibody concentrations, the specific binding was titrated by ELISA or FACS.

### Production of Anti-CD70 Antibodies:

Full IgG anti-CD70 lead antibodies (1A4, 2A4, 1H8, 2D2, 2E7 and 2A4P0L1) and the reference parent antibody (69A7) and another reference antibody, 1 F6 (Vorsetuzumab, see U.S. Pat. No. 7,491,390), were constructed from the 6 leads and two controls so as to have their human heavy and light variable regions connected to the human constant regions IgG1 and Kappa, respectively. (The VH and VL sequences of 1F6 antibody are shown in SEQ ID NOs: 19 and 20, respectively.) Briefly, the Kozak consensus sequence “GCCGCCACC” (SEQ ID NO:31) and the signal peptide “MGWSCIILFLVATATGVHS” (SEQ ID NO: 32) were inserted at the 5′ terminal of the gene construct for adequate translation and antibody secretion. The final DNA coding sequence of heavy and light chains was optimized and synthesized and constructed in the vector pcDNA3.4.

The resulting plasmids were transiently transfected into ExpiCHO-S cells using a ExpiCHO™ Expression System (Thermo, ExpiFectamine™ CHO Transfection Kit, Cat #A29129) based on a standard ExpiFectamine CHO Transfection procedure (Gibco, A29129) in spinner flasks. The suspensions of transient transfections were incubated for 10 days and then the cleared supernatants were purified using Protein A columns.

Antibodies were purified from cleared cell culture supernatants using Protein A chromatography (Protein A resin slurry, 4.5 mL, Bogen, Cat #18-0010-02). Briefly, supernatants were prepared for affinity chromatography and loaded onto the columns and allowed to flow completely through the resin. The columns were washed with binding buffer containing 0.15M NaCl and 0.2M PB, PH7.0. Antibody was eluted with elution buffer containing 0.15M NaCl, 0.1M Glycine and 0.2M PB, PH 3.0. Fractions were collected and neutralized by the addition of 1/10 volume of 1M Tris, PH9.0. The fractions were dialyzed for 2 hours against 1×PBS. Purified antibody was quantified by absorbance at A280. Samples from each step of the protein A chromatography were applied onto 12% SDS-PAGE gels for reduced and non-reduced electrophoresis. The hydrophobicity was assessed with hydrophobic interaction chromatography (HIC) on a TSK gel Butyl-NPR, 4.6×100 mm with Butyl-NPR (Tosoh corporation) using a Waters HPLC 2695 system.

The expression levels following purification are shown in Table 4. Antibody clone 2A4 had a higher expression level; clone 1A4, 2D2 and 2E7 had medium expression levels and clone 2A4P0L1 and 1H8 had lower expression levels.

The hydrophobicity analyses are shown in Table 5. Clone 2D2 and 2A4 had the similar hydrophobicity with the parent antibody, 69A7; clone 2A4P0L1, 1 H8 and 2E7 were slightly more hydrophobic than the parent antibody 69A7.

TABLE 4
Comparison of antibodies expression levels

| Sample | Trans | Conc. | Vol. | Quantity |
|---|---|---|---|---|
| Name | Vol (ml) | (mg/ml) | (ml) | (mg) |
| 1A4 | 60 | 1.965 | 6.5 | 12.77 |
| 2A4 | 60 | 3.356 | 6.5 | 21.81 |
| 2A4P0L1 | 60 | 0.663 | 6.5 | 4.30 |
| 1H8 | 60 | 0.541 | 6.5 | 3.52 |
| 2D2 | 60 | 1.993 | 6.5 | 12.95 |
| 2E7 | 60 | 1.782 | 6.5 | 11.58 |
| 69A7 | 60 | 2.78 | 10.5 | 29.19 |

TABLE 5
Hydrophobicity of anti-CD70 antibodies

| Sample name | Retention time (min) |
|---|---|
| 2A4P0L1 | 9.56 |
| 2A4 | 9.06 |
| 2D2 | 8.81 |
| 1H8 | 10.24 |
| 2E7 | 10.19 |
| 69A7 | 8.83 |

### Antibody Binding Characterization by ELISA

The binding specificity of the CD70 antibodies was tested by ELISA according to a standard protocol. Briefly, 96-well micro-plates were coated with 2 μg/ml of human or cynomolgus CD70 recombinant protein in PBS at 100 μl per well and incubated overnight at 4° C. The plates were washed twice with TBS+0.5% Tween20. 200 μL of blocking buffer (2% BSA in PBS) was added to each well, and the plates were incubated at 37° C. for 2 hours. The plates were washed using the wash buffer mentioned above. Serially diluted antibodies were added to the ELISA plates using 100 μl per well, and the plates were incubated for 1 hour at room temperature. Then plates were washed 3 times. HRP conjugated anti-human Fc antibody solution (Sigma, I18885-2ML, diluted with blocking buffer) was added to the plates using 100 μl per well. The plates were incubated at room temperature for 1 hour and then washed 3 times. Then the TMB solution was added to plates using 100 μl per well and the plates were placed at RT for 5-15 mins. Then the stop solution (2M H2SO4) was added using 50 μl per well. The absorbance was measured at A450 and A630.

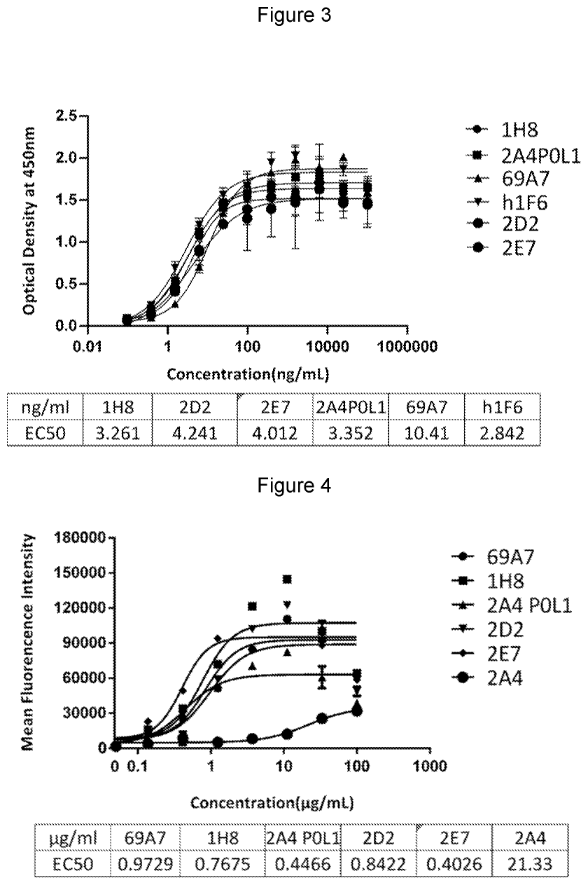

The results are shown in FIG. 2 and FIG. 3. The half maximal effective concentration (EC₅₀) value of antibody 2E7 is 1.5 times more than that of antibody 69A7. Antibodies 2E7, 1H8 and 2D2 had better cross binding activity to the cyno-antigen than antibody 69A7, similar with h1 F6.

### Determination of Antibody Binding Affinity by Flow Cytometry

The lead antibodies were tested for binding to renal carcinoma cells and glioblastoma cells expressing CD70 on the cell surface by flow cytometry. Cell lines 786-O (ATCC® CRL-1932™, provided by COBIOER), Caki-1 (ATCC® HTB-46™, provided by COBIOER), U251 and DBTRG-05MG (ATCC® CRL-2020™, provided by COBIOER) were each tested for antibody binding. 786-O cells were cultured with RPMI 1640 medium (Gibco, Cat #11875093) containing of 10% FBS (Gibco, Cat #10099141) while Caki-1 cells were cultured with McCoy's 5a Modified medium (Gibco, Cat #16600082) containing 10% FBS. U251 cells were cultured with MEM medium containing of 10% FBS and 1% NEAA+1 mM sodium pyruvate. DBTRG-05MG cells were cultured with DMEM medium (Gibco, Cat #C11995500BT) containing of 10% FBS. Each of the lead antibodies and the controls were incubated for 30 mins with the different cell lines (3×10⁵ cells per well) in 0.2 ml FACS buffer (1×PBS with 0.1% BSA) at 4° C. Then, the cells were pelleted, washed, and incubated at 4° C. for 30 mins with 100 μl of 1:200 diluted PE-conjugated anti human Fc (Abcam, Ab98596) in FACS buffer. The cells were pelleted again, washed with PBS, resuspended in FACS buffer and analyzed by flow cytometer (Beckman, CytoFLEX).

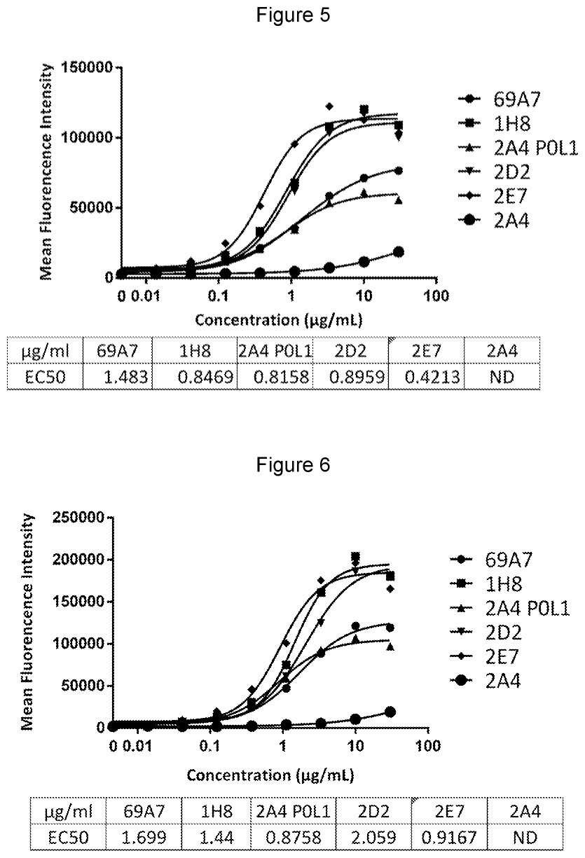

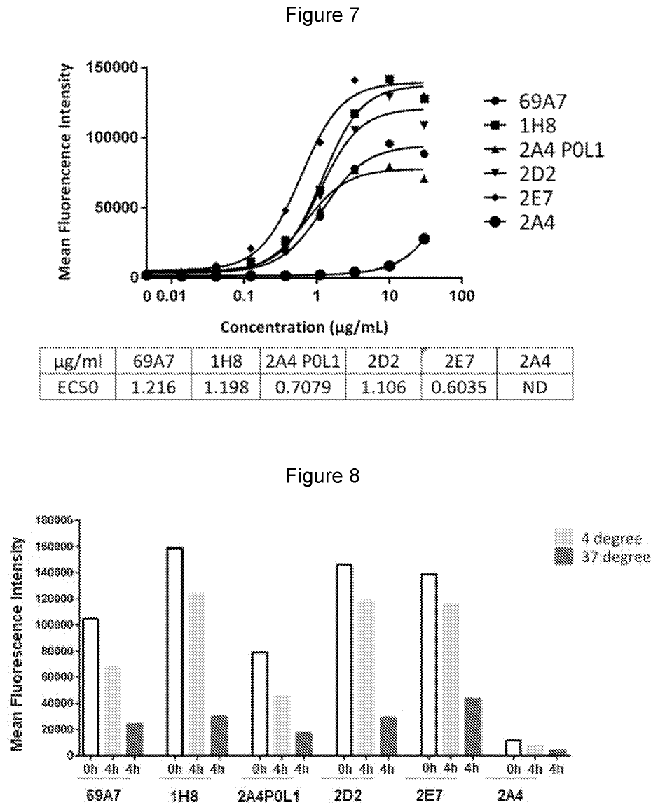

The EC50s of anti-CD70 antibodies on different cell lines results are shown in Table 6 and FIGS. 4 to 7. The results obtained by flow cytometry analyses confirmed the anti-CD70 antibodies bind to renal cell line 786-O and Caki-1 cells, and bind to glioblastoma U251 and DBTRG-05MG cells. The EC50 of antibody 2E7 was 1.8˜3.5 times more than that of antibody 69A7, the EC50 of antibody 2A4P0L1 was 1.7-2.2 times more than that of antibody 69A7. Antibodies 2H8 and 2D2 had slightly higher binding ability to the cell lines than that of antibody 69A7.

TABLE 6
EC₅₀'s of anti-CD70 antibodies
binding to different tumor cell lines
Results of anti-CD70 antibodies binding
to human renal cells and glioblastoma
cells expressing CD70 on cell surface

| Tumor cell line | EC50 (nM) | |
|---|---|---|
| 786-O | 69A7 | 6.49 |
| | 1H8 | 5.12 |
| | 2A4 P0L1 | 2.98 |
| | 2D2 | 5.62 |
| | 2E7 | 2.69 |
| | 2A4 | 142.27 |
| Caki-1 | 69A7 | 9.89 |
| | 1H8 | 5.65 |
| | 2A4 P0L1 | 5.44 |
| | 2D2 | 5.98 |
| | 2E7 | 2.81 |
| | 2A4 | 284.01 |
| DBTRG-05MG | 69A7 | 11.33 |
| | 1H8 | 9.60 |
| | 2A4 P0L1 | 5.84 |
| | 2D2 | 13.73 |
| | 2E7 | 6.11 |
| | 2A4 | 494.18 |
| U251 | 69A7 | 8.11 |
| | 1H8 | 7.99 |
| | 2A4 P0L1 | 4.72 |
| | 2D2 | 7.38 |
| | 2E7 | 4.03 |
| | 2A4 | NA |

### Example 4: Internalization of the Anti-CD70 Antibodies

The lead antibodies and controls were tested for their ability to internalize into 0070-expressing renal carcinoma cells 786-O and Caki-1 cells using a FACS immune-fluorescence staining assay.

Briefly, 2×10⁵ cells were harvested from a tissue culture flask by treatment with 0.25% Trypsin/EDTA and then incubated with 10 μg/ml of the lead antibodies or control antibodies in FACS buffer (1×PBS with 0.1% BSA) for 30 mins at 4° C. The cells were washed at 4° C. to remove unbound antibodies and kept on ice or shifted to 37° C. At the set time points (0 h, 4 h, 24 h), cells were incubated with PE-conjugated anti human Fc (Abcam, Ab98596) for 30 mins at 4° C. and then analyzed by flow cytometry. The internalization ratio was calculated by subtracting the 37° C. MFI from the 4° C. MFI, and then compared with 4° C. MFI.

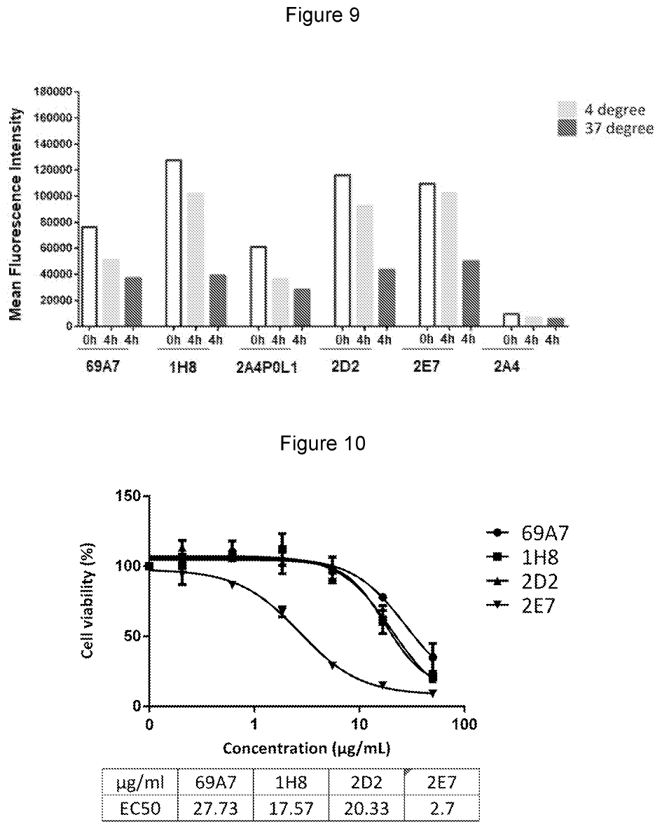

The results are shown in Table 7 and FIGS. 8 and 9. The results show changes in surface levels of anti-CD70 antibodies on 786-O and Caki-1 cell lines kept at 4° C. or 37° C. for the course of the 4 h study. Surface levels of antibodies declined significantly in cells shifted to 37° C. over the course of the assay. Based on the cell binding affinity, the results demonstrate that anti-CD70 antibodies 1H8, 2D2 and 2E7 had a higher absolute amount of antibody internalized into cell than the parent antibody 69A7.

TABLE 7
Comparison of anti-CD70 antibodies
internalization on renal tumor cell

| | Internalization rate on | |
|---|---|---|
| | different cell lines (%) | |
| Samples | 786-O | Caki-1 |
| 69A7 | 64 | 27 |
| 1H8 | 76 | 62 |
| 2A4P0L1 | 61 | 22 |
| 2D2 | 76 | 53 |
| 2E7 | 62 | 51 |
| 2A4 | 49 | 20 |

### Example 5: Measurement of Affinity Binding of the CD70 Antibodies to CD70

Immobilization of CD70 Antigen onto a CM5 Sensor Chip.

The immobilization of antigen CD70 ECD was performed at 25° C. with HBS-EP as the running buffer. Sensor chip surface of flow cells 1, 4 were activated by freshly mixed 50 mmol/L N-Hydroxy succinimide (NHS) and 200 mmol/L 1-ethyl-3-(3-dimethylaminopropyl) carbodiimide hydrochloride (EDC) for 420s (10 μL/min). Afterwards, CD70 antigen diluted in 10 mmol/L NaAC (pH 4.5) was injected into flow cell 4 to achieve conjugation of the appropriate Response Unit, while flow cell 1 was set as a blank. After the amine coupling reaction, the remaining active coupling sites on chip surface were blocked by 420s injection of 1 mol/L ethanolamine hydrochloride.

### Affinity Measurement of CD70 Antibody Binding to the CD70 ECD.

The assay was performed at 25° C. and the running buffer was HBS-EP. Diluted antibodies were injected over the surface of flow cells 1 and 4 during the association phase, followed by injecting running buffer as the dissociation phase. All the data were processed using Biacore T200 Evaluation software version 3.1. Flow cell 1 and a blank injection of buffer in each cycle were used as a double reference for Response Units subtraction. Kinetic data of the interaction between the antibodies and CD70 antigen were obtained through affinity measurement.

The data are summarized in Table 8. Antibody 2E7 has a 22-fold greater affinity to CD70 antigen than the parent antibody 69A7, and has 2-3-fold greater affinity to CD70 antigen than the reference antibody h1 F6. The affinity of other antibodies, 1H8, 2D2, 2A4 and 2A4P0L1, to CD70 antigen was 3-8-fold improved compared to the affinity of parent antibody 69A7.

TABLE 8
Affinity measurement of antibody binding to CD70

| | | ka | | | Rmax | Chi² |
|---|---|---|---|---|---|---|
| Ligand | Analyte | (1/Ms) | kd (1/s) | KD (M) | (RU) | (RU²) |
| CD70 | 2E7 | 7.54E+04 | 1.06E−03 | 1.40E−08 | 32.94 | 3.48E−01 |
| | 1H8 | 2.56E+04 | 1.12E−03 | 4.37E−08 | 38.81 | 6.46E−01 |
| | 2D2 | 1.17E+04 | 1.05E−03 | 8.96E−08 | 38.83 | 1.38E−01 |
| | 2A4P0L1 | 5.10E+04 | 1.97E−03 | 3.86E−08 | 45.47 | 1.54E+00 |
| | h1F6 | 4.51E+04 | 2.36E−03 | 5.23E−08 | 19.27 | 7.96E−01 |
| | 69A7 | 6.33E+03 | 1.98E−03 | 3.13E−07 | 38.01 | 7.29E−01 |

### Example 6: Assessment of Cell Killing by the Anti-CD70 Antibodies Conjugated to a Cytotoxin—on a Renal Cell Carcinoma Cell Line

Anti-CD70 antibodies conjugated to a cytotoxin were tested for the ability to kill CD70+ renal cell carcinoma cell lines in a cell proliferation assay.

Anti-CD70 conjugates were prepared as follows: The pH of a CD70 antibody solution was adjusted within the range of pH 7.0-7.5 by adding 0.5M sodium phosphate dibasic. 0.5M EDTA was added to achieve a final EDTA concentration of 5 mM in the antibody solution. 10 mM TCEP (Tris(2-chloroethyl) phosphate solution was added to achieve desired TCEP/mAb molar ratio. The reduction was kept at RT for 90 mins. DMSO was then added to achieve a 10% v/v concentration. Drug-linked Mc-VC-PAB-MMAE (maleimidyl caproyl-valine-citrulline-para-aminobenzoyl MMAE) was dissolved in DMSO to achieve a final concentration of 10 mM and added in the reaction solution in a molar excess of 30-50% compared to the moles of cysteine thiols available. The conjugation reaction was placed at RT for 30 mins. To quench the reaction, NAC (N-Acetyl-L-cysteine) stock solution was added to achieve an NAC/Mc-VC-PAB-MMAE molar ratio of 5. The quenching reaction was placed at RT for 15 mins. Purification was carried out by PD10 column.

### Cytotoxicity on Renal Tumor Cells

The renal carcinoma cancer cell line 786-O was seeded at 400 cells per well for 24 hours. Antibody conjugates, prepared as described above, were added to the wells at a starting concentration of 30 μg/ml with 3 fold serial dilutions. The plates were allowed to incubate for 96 hours. After 90 hours, 40 μl CTG (Promega, Cat #G7572) per well was added to the plates and luciferase read after 5 mins. The percentage of growth inhibition was calculated relative to untreated cells.

The results are shown in FIG. 10. The results demonstrate that the anti-CD70 conjugates 1H8-ADC, 2D2-ADC and 2E7-ADC were cytotoxic to the renal carcinoma cancer cells. The IC50 value for the antibody conjugates shows that 2E7-ADC had a much better cell growth inhibitory (more than 10 times) than the parent antibody conjugate (69A7-ADC).

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

Various publications, including patents, patent application publications, and scientific literature, are cited herein, the disclosures of which are incorporated by reference in their entireties for all purposes.

### Example 7: Affinity Data of 2E7/69A7 to Species CD70 Tested by Biolayer Interferometry (BLI)

Recombinant proteins consisting of human, rat, or mouse CD70 extra-cellular domain (ECD) linked to His tag was purchased (from ACRO systems). 69A7 and 2E7 (20 nM) were immobilized on anti-human IgG Fc biosensor tips (ForteBio). Binding assays using one concentration (100 nM) of recombinant protein in solution were performed using Octet RED (ForteBio). Association time was set at 180s and dissociation time was set at 300s. Binding affinity was calculated using ForteBio Data Acquisition 6.3 software. Affinity was derived by fitting the kinetic data to a 1:1 Langmuir binding model utilizing global fitting algorithms. 69A7 and 2E7 demonstrated high binding affinity to human CD70 with the equilibrium dissociation constant (KD) of 2.9 and 0.97 nM, respectively. 69A7 and 2E7 displayed no cross-reactivity to rat and mouse CD70 (Table 9). Binding assays for 2E7 using multiple dilutions (from 200 nM down to 3.13 nM) of species' recombinant CD70 ECD proteins in solution were also performed using the same method. 2E7 displayed high binding affinity to human and cyno CD70 with the KD of 0.81 and 0.39 nM, respectively. 2E7 had no cross-reactivity to rat or mouse CD70 (Table 10).

TABLE 9
Affinity data of 69A7/2E7 to species CD70 tested by BLI

| | Loading | Loading | | | | | |
|---|---|---|---|---|---|---|---|
| Sample | Sample | Conc. | Conc. | Res- | KD | ka | kdis |
| ID | ID | (ug/ml) | (nM) | ponse | (M) | (1/Ms) | (1/s) |
| Hu | 69A7 | 3. | 100. | 0.3334 | 2.888E− | 3.387E05 | 9.782E− |
| CD70 | | | | | 09 | | 04 |
| Rat | 69A7 | 3. | 100. | *0.0198 | 1.413E− | 9.425E04 | 1.332E− |
| CD70 | | | | | 07 | | 02 |
| MS | 69A7 | 3. | 100. | *0.0113 | 1.002E− | 1.494E03 | 1.497E− |
| CD70 | | | | | 05 | | 02 |
| Hu | 2E7 | 3. | 100. | 0.5157 | 9.787E− | 3.650E05 | 3.572E− |
| CD70 | | | | | 10 | | 04 |
| Rat | 2E7 | 3. | 100. | *0.0125 | 6.401E− | 4.641E03 | 2.971E− |
| CD70 | | | | | 06 | | 02 |
| MS | 2E7 | 3. | 100. | *0.0045 | 8.681E− | 3.250E03 | 2.821E− |
| CD70 | | | | | 06 | | 02 |

*Response below range of quantification

TABLE 10
Affinity data of 2E7 to species CD70 tested by BLI

| | Loading | Loading | | | | | |
|---|---|---|---|---|---|---|---|
| Sample | Sample | Conc. | Conc. | | | | kdis |
| ID | ID | (ug/ml) | (nM) | Response | KD (M) | ka (1/Ms) | (1/s) |
| HU | 2E7 | 3. | 200. | 0.825 | 8.068E− | 3.143E05 | 2.536E− |
| CD70 | | | | | 10 | | 04 |
| HU | 2E7 | 3. | 100 | 0.7695 | 8.068E− | 3.143E05 | 2.536E− |
| CD70 | | | | | 10 | | 04 |
| HU | 2E7 | 3. | 50. | 0.6227 | 8.068E− | 3.143E05 | 2.536E− |
| CD70 | | | | | 10 | | 04 |
| HU | 2E7 | 3. | 25. | 0.5319 | 8.068E− | 3.143E05 | 2.536E− |
| CD70 | | | | | 10 | | 04 |
| HU | 2E7 | 3. | 12.5 | 0.3519 | 8.068E− | 3.143E05 | 2.536E− |
| CD70 | | | | | 10 | | 04 |
| HU | 2E7 | 3. | 6.25 | 0.2136 | 8.068E− | 3.143E05 | 2.536E− |
| CD70 | | | | | 10 | | 04 |
| HU | 2E7 | 3. | 3.13 | 0.1732 | 8.068E− | 3.143E05 | 2.536E− |
| CD70 | | | | | 10 | | 04 |
| CYNO | 2E7 | 3. | 200. | 0.4991 | 3.900E− | 6.178E05 | 2.409E− |
| CD70 | | | | | 10 | | 04 |
| CYNO | 2E7 | 3. | 100. | 0.4825 | 3.900E− | 6.178E05 | 2.409E− |
| CD70 | | | | | 10 | | 04 |
| CYNO | 2E7 | 3. | 50. | 0.4136 | 3.900E− | 6.178E05 | 2.409E− |
| CD70 | | | | | 10 | | 04 |
| CYNO | 2E7 | 3. | 25. | 0.3702 | 3.900E− | 6.178E05 | 2.409E− |
| CD70 | | | | | 10 | | 04 |
| CYNO | 2E7 | 3. | 12.5 | 0.2927 | 3.900E− | 6.178E05 | 2.409E− |
| CD70 | | | | | 10 | | 04 |
| CYNO | 2E7 | 3. | 6.25 | 0.204 | 3.900E− | 6.178E05 | 2.409E− |
| CD70 | | | | | 10 | | 04 |
| CYNO | 2E7 | 3. | 3.13 | 0.1275 | 3.900E− | 6.178E05 | 2.409E− |
| CD70 | | | | | 10 | | 04 |
| RAT | 2E7 | 3. | 200. | *0.0054 | 1.755E− | 5.905E06 | 1.037E− |
| CD70 | | | | | 09 | | 02 |
| RAT | 2E7 | 3. | 100. | *0.0271 | 1.755E− | 5.905E06 | 1.037E− |
| CD70 | | | | | 09 | | 02 |
| RAT | 2E7 | 3. | 50. | *0.0126 | 1.755E− | 5.905E06 | 1.037E− |
| CD70 | | | | | 09 | | 02 |
| RAT | 2E7 | 3. | 25. | *0.0069 | 1.755E− | 5.905E06 | 1.037E− |
| CD70 | | | | | 09 | | 02 |
| RAT | 2E7 | 3. | 12.5 | *−2.762E−03 | 1.755E− | 5.905E06 | 1.037E− |
| CD70 | | | | | 09 | | 02 |
| RAT | 2E7 | 3. | 6.25 | *0.0192 | 1.755E− | 5.905E06 | 1.037E− |
| CD70 | | | | | 09 | | 02 |
| RAT | 2E7 | 3. | 3.13 | *0.0012 | 1.755E− | 5.905E06 | 1.037E− |
| CD70 | | | | | 09 | | 02 |
| MOUSE | 2E7 | 3. | 200. | *0.0012 | 1.032E− | 1.170E05 | 1.208E− |
| CD70 | | | | | 06 | | 01 |
| MOUSE | 2E7 | 3. | 100. | *0.0111 | 1.032E− | 1.170E05 | 1.208E− |
| CD70 | | | | | 06 | | 01 |
| MOUSE | 2E7 | 3. | 50. | *0.0063 | 1.032E− | 1.170E05 | 1.208E− |
| CD70 | | | | | 06 | | 01 |
| MOUSE | 2E7 | 3. | 25. | *−3.009E−03 | 1.032E− | 1.170E05 | 1.208E− |
| CD70 | | | | | 06 | | 01 |
| MOUSE | 2E7 | 3. | 12.5 | *−1.516E−03 | 1.032E− | 1.170E05 | 1.208E− |
| CD70 | | | | | 06 | | 01 |
| MOUSE | 2E7 | 3. | 6.25 | *−5.879E−03 | 1.032E− | 1.170E05 | 1.208E− |
| CD70 | | | | | 06 | | 01 |
| MOUSE | 2E7 | 3. | 3.13 | *−6.014E−03 | 1.032E− | 1.170E05 | 1.208E− |
| CD70 | | | | | 06 | | 01 |

*Response below range of quantification

### Example 8: 2E7 Binding to Cells, Raji and MCF-7

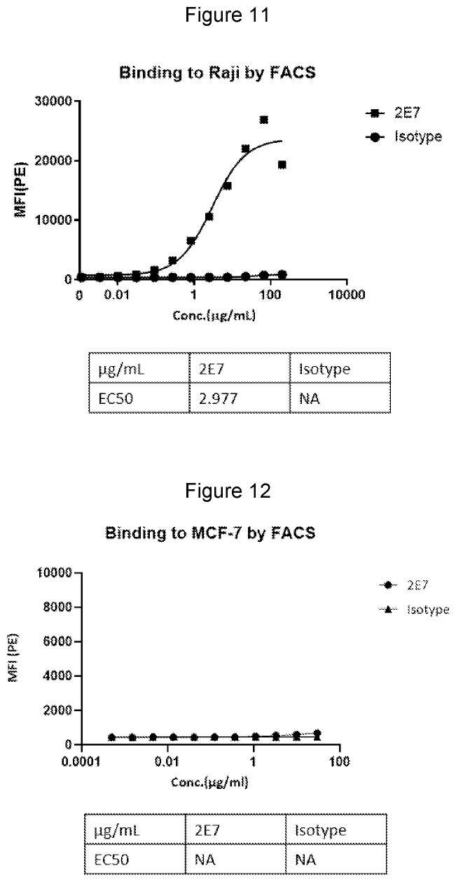

Binding activity of 2E7 or the isotype control with a target cell line (Raji) or a cell line (MCF-7) that has negligible level of CD70 expression were evaluated by flow cytometry (Beckman, Cytoflex). 3×10⁵ cells per well were seeded on a 96-well V-bottomed plate and incubated with 100 μl of 2E7 in serial dilutions. After 30 min incubation at 4° C., cells were washed twice with PBS, stained with 100 μl of 1:200 diluted PE-conjugated anti-human Fc in FACS buffer (1×PBS containing 1% BSA), then incubated at 4° C. for 30 min. Cells were finally washed two times with PBS and analyzed by flow cytometric analysis. 2E7 displayed strong binding activity to human CD70-expressing cell line, Raji, with an EC50 of −19 nM (FIG. 11), while displaying no binding to the CD70-negative cell line, MCF-7 (FIG. 12), demonstrating specificity of the interaction.

### Example 9: 2E7 Internalization Using Additional Cell Lines (Raji, MCF7) in Time Course

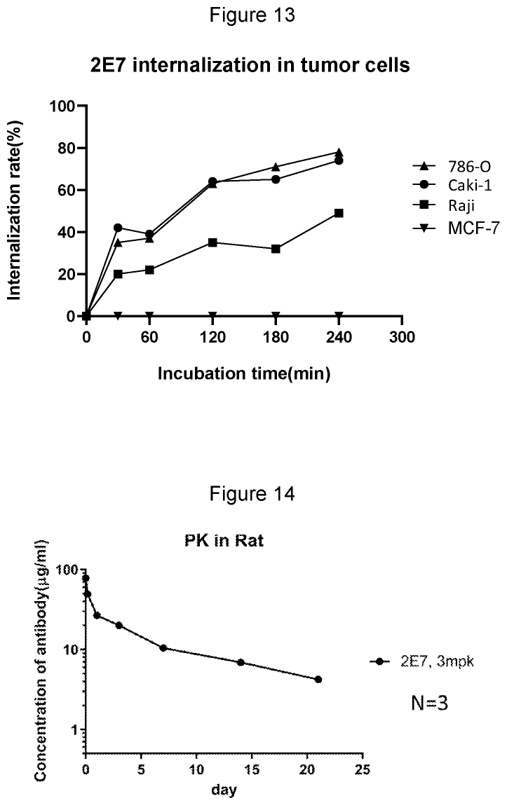

Four cell lines (786-), Caki-1, Raji, MCF-7) were utilized in the internalization assay. Target (CD70) copy numbers were determined via the QIFIKIT (DAKO, K0078). Briefly, cells were labeled with a primary mouse monoclonal antibody against CD70. Cells, Set-up beads, Calibration beads (from the kit) were then labeled in parallel with a fluorescein-conjugated anti-mouse secondary antibody. The fluorescence is correlated with the number of bound primary antibody molecules on the cells and on the beads. Samples were subsequently analyzed on the flow cytometer and copy number determined based on the calibration curve (Table 11). For internalization assay, 3×10⁵ cells were incubated at 4° C. for 30 min with 10 g/ml 2E7 in FACS buffer (1×PBS containing 0.1% BSA). Cells were washed at 4° C. to remove unbound material and kept on ice or shifted to 37° C. for different lengths of time. At progressive timepoints (1, 0.5, 1, 2, 3, 4 hr) cells were stained with PE-conjugated anti-human Fc for 30 min at 4° C. and analyzed by flow cytometry. Internalization rate were calculated by subtracting the mean fluorescence intensity (MFI) of cell surface-bound antibody at 37° C. at each time point from the MFI of cell surface-bound antibody at 4° C. at time 0, then divided by the MFI of the cell surface-bound antibody at 4° C. at time 0. 2E7 displayed rapid internalization on CD70-expressing cell lines (786-O, Caki-1, Raji), and no internalization on the CD70-negative cell line (MCF-7) (FIG. 13).

TABLE 11
Target (CD70) copy numbers in cell lines

| | | Copy number |
|---|---|---|
| Cell line | Tumor type | (X10³ per cell) |
| 786-O | renal | 149 |
| Caki-1 | renal | 119 |
| Raji | lymphoma | 62 |
| MCF-7 | breast | 3.8 |

### Example 10: 2E7 rat PK

2E7 was administered via intravenous infusion at 3 mg/kg to male Sprague Dawley rats (n=3 per group). Orbital blood was sampled from each rat at various time points post dosing. Circulating concentrations of 2E7 were analyzed by an ELISA assay (ProfoundBio) and calculated using the GraphPad Prism 6 software. 2E7 displayed a stable plasma PK in rat that is characteristic of IgG1 antibodies (FIG. 14).

### Example 11: 2E7 Conjugates: In Vitro Cytotoxicity

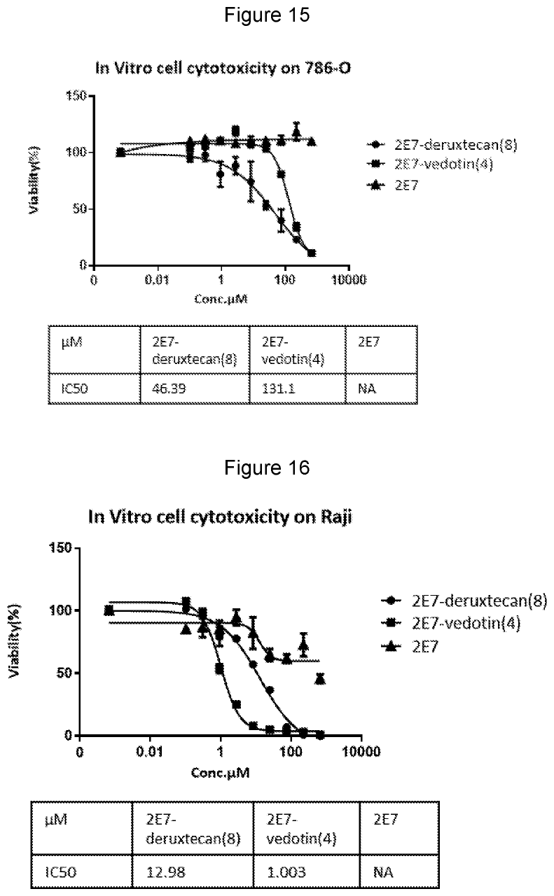

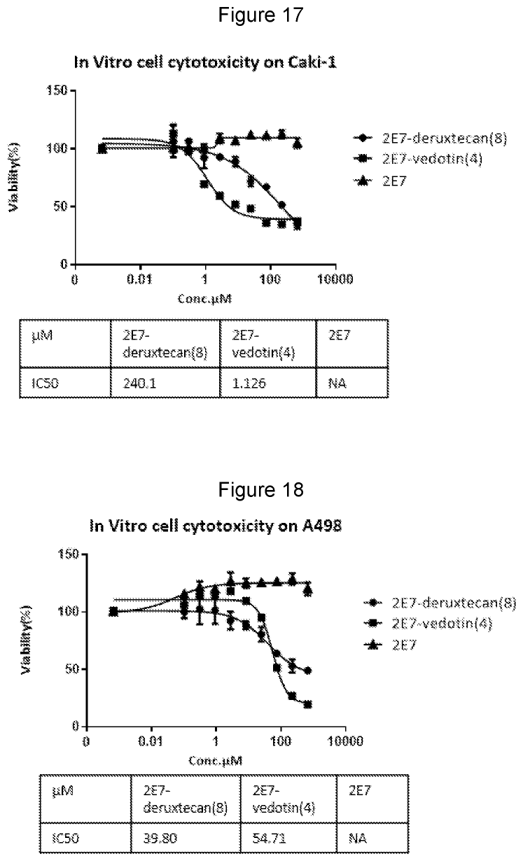

Two 2E7-conjugates were utilized in the study (Table 12). For preparation of 2E7-deruxtecan: 2 mL of antibody (10 mg/mL) in 50 mM sodium phosphate buffer containing 5 mM EDTA (pH=6.9) was added to the aqueous solution of 10 mM TCEP HCl (Tris(2-carboxyethyl) phosphine HCl), at the molar ratio of 8.0 (TCEP to mAb). The reducing reaction proceeded for 2 hr at 25° C. Deruxtecan (dissolved in DMSO at a concentration of 20 mg/mL) was added to the reduced antibody at a molar ratio of 12 (deruxtecan/mAb). The coupling reaction was stirred for 8 hr at 25° C. The excess deruxtecan and the impurities were removed by ultrafiltration with 50 mM sodium phosphate buffer. The ADC was stored in 20 mM histidine buffer containing 6% sucrose and 0.02% (w/V) Tween 20 by UFDF. The purity measured by SEC-HPLC was 97.2% and DAR value measured by LC-MS was 7.5. For preparation of 2E7-vedotin, 2 mL of antibody (10 mg/mL) in 50 mM sodium phosphate buffer containing 5 mM EDTA (pH=6.9) was added the aqueous solution of 10 mM TCEP HCl (Tris(2-carboxyethyl) phosphine HCl), at the molar ratio of 2.2 (TCEP to mAb). The reducing reaction proceeded for 2 hr at 25° C. Vedotin (dissolved in DMSO at a concentration of 20 mg/mL) was added to reduced antibody at a molar ratio of 5.0 (vedotin/mAb). The coupling reaction was stirred for 2 hr at 25° C. The excess vedotin and the impurities were removed by ultrafiltration with 50 mM sodium phosphate buffer. The ADC was stored in 20 mM histidine buffer containing 6% sucrose and 0.02% (w/V) Tween 20 by UFDF. The purity measured by SEC-HPLC was 97.4% and DAR value measured by HIC-HPLC was 3.9. For the in vitro cytotoxicity study: one day prior to adding 2E7 conjugates, cells were harvested and plated into 96-well solid white flat bottom plates. The next day cells were exposed to the test article at a concentration range of 670 nM to 0.00067 nM. Plates were incubated at 37° C. for 96 h. After that, 40 μl Cell-tire Glo (CTG) per well was added to the plates with luciferase reading collected at 5 min after incubation, and analyzed by Microplate readers. All readings were normalized as percentage of viable cells in the untreated control wells and the IC50 values were calculated by Prism software. 2E7-deruxtecan and 2E7-vedotin, but not 2E7, produced cytotoxic effect on all four cell lines tested (FIG. 15-18).

TABLE 12
2E7 conjugates utilized in the study

| Benchmarking | |
|---|---|
| ADC (DAR) | Linker-drug |
| 2E7-deruxtecan(8) | mc-GGFG-Dxd |
| 2E7-vedotin(4) | mc-vc-PAB-MMAE |

### Example 12: 2E7 Conjugates: In Vivo Efficacy in Cell Line-Derived Xenograft (CDX) Models

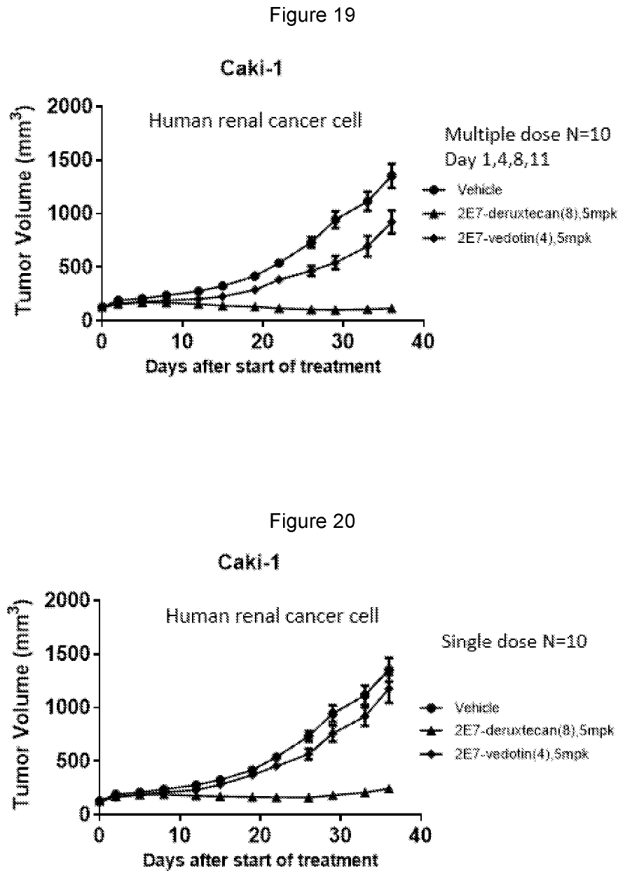

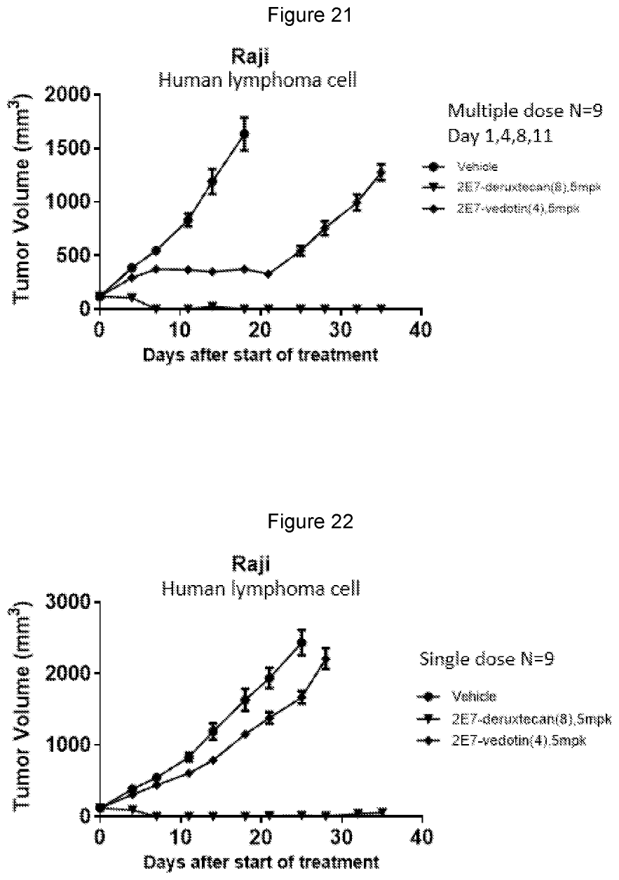

Antitumor activity of 2E7 in conjugate with the benchmarking linker-drugs (Table 12) was evaluated in CDX models. Female BALB/c nude mice were inoculated subcutaneously at right flank with Caki-1 cells (ATCC, HTB-46, 3×10⁶ in 0.2 mL cell suspension) or Raji cells (From Betapharma, 5×10⁶ in 0.1 mL cell suspension) for tumor development. Five to eight days after tumor inoculation, mice with average tumor size 120˜130 mm³ were selected and assigned into treatment groups for each model using stratified randomization based upon their tumor volumes (n=9˜10 mice per group). The treatment initiated one day after randomization (randomization day defined as DO) and was in either single-dose (on day1) or multiple-dose (day1/day4/day8/day11) regimen via intravenous infusion of the 2E7 conjugates at 5 mg/kg. Tumor size and body weight were measured twice a week in two dimensions using a caliper, and the volume was expressed in mm³ using the formula: V=0.5 a×b² where a and b are the long and short diameters of the tumor, respectively. Tumor volume exceeding 2000 mm³ was defined as endpoint. Animal body weight was monitored as an indirect measure of toxicity. No mice showed significant weight loss in any of the study groups. There were no morbidity or death in the treatment duration. Compared to vehicle control group, treatment with 2E7-deruxtecan(8) produced marked inhibition on tumor growth, in the multiple or single-dose models, with the Caki-1 or Raji cells; 2E7-vedotin(4) exerted low-to-moderate antitumor activity in these models (FIG. 19-22).

1.-83. (canceled)

84. A binding agent comprising:
a heavy chain variable (VH) region and a light chain variable (VL) region, the VH region comprising complementarity determining regions HCDR1, HCDR2 and HCDR3, and the VL region comprising LCDR1, LCDR2 and LCDR3, wherein the VH and VL regions have amino acid sequences that are selected from the sets of amino acid sequences set forth in:
a. SEQ ID NO:21, SEQ ID NO:22, SEQ ID NO:13, SEQ ID NO:24, SEQ ID NO:25 and SEQ ID NO:26;
b. SEQ ID NO:21, SEQ ID NO:22, SEQ ID NO:14, SEQ ID NO:24, SEQ ID NO:25 and SEQ ID NO:26;
c. SEQ ID NO:21, SEQ ID NO:22, SEQ ID NO:15, SEQ ID NO:24, SEQ ID NO:25 and SEQ ID NO:26;
d. SEQ ID NO:21, SEQ ID NO:22, SEQ ID NO:23, SEQ ID NO:24, SEQ ID NO:25 and SEQ ID NO: 18; or
e. SEQ ID NO:16, SEQ ID NO: 17, SEQ ID NO:23, SEQ ID NO:24, SEQ ID NO:25 and SEQ ID NO:26, respectively.

85. The binding agent of claim 84, wherein (a) the HCDR1 comprises the amino acid sequence set forth in SEQ ID NO: 21, (b) the HCDR2 comprises the amino acid sequence set forth in SEQ ID NO: 22, (c) the HCDR3 comprises the amino acid sequence set forth in SEQ ID NO: 15, (d) the LCDR1 comprises the amino acid sequence set forth in SEQ ID NO: 24, (e) the LCDR2 comprises the amino acid sequence set forth in SEQ ID NO: 25, and (f) the LCDR3 comprises the amino acid sequence set forth in SEQ ID NO: 26.

86. The binding agent of claim 84, wherein the VH and VL regions have amino acid sequences that are selected from the pairs of amino acid sequences set forth in:
a. SEQ ID NO:3 and SEQ ID NO:4;
b. SEQ ID NO:5 and SEQ ID NO:6;
c. SEQ ID NO:7 and SEQ ID NO:8;
d. SEQ ID NO:9 and SEQ ID NO:10; or
e. SEQ ID NO:11 and SEQ ID NO:12, respectively,
wherein the VH and VL regions comprise framework regions that are optionally modified with from 1 to 8 amino acid substitutions, deletions or insertions.

87. The binding agent of claim 86, wherein the VH and VL regions comprise framework regions that are optionally modified with from 1 to 4 amino acid substitutions, deletions or insertions.

88. The binding agent of claim 84, wherein the VH and VL regions have amino acid sequences that are selected from the pairs of amino acid sequences set forth in:
a. SEQ ID NO:3 and SEQ ID NO:4;
b. SEQ ID NO:5 and SEQ ID NO:6;
c. SEQ ID NO:7 and SEQ ID NO:8;
d. SEQ ID NO:9 and SEQ ID NO:10; or
e. SEQ ID NO:11 and SEQ ID NO:12, respectively.

89. The binding agent of claim 88, wherein the VH and VL regions have amino acid sequences that are selected from the pairs of amino acid sequences set forth in:
a. SEQ ID NO:5 and SEQ ID NO:6;
b. SEQ ID NO:7 and SEQ ID NO:8; or
c. SEQ ID NO:9 and SEQ ID NO:10, respectively.

90. The binding agent of claim 89, wherein the VH and VL regions have amino acid sequences that are selected from the pairs of amino acid sequences set forth in:
a. SEQ ID NO:7 and SEQ ID NO:8; or
b. SEQ ID NO:9 and SEQ ID NO:10, respectively.

91. The binding agent of claim 90, wherein the VH has the amino acid sequence set forth in SEQ ID NO: 7 and the VL has the amino acid sequence set forth in SEQ ID NO: 8.

92. The binding agent of claim 84, wherein the VH and the VL regions comprise human framework regions.

93. The binding agent of claim 84, wherein the binding agent is an antibody or antigen-binding portion thereof.

94. The binding agent of claim 93, wherein the binding agent is a monoclonal antibody, a Fab, a Fab′, an F(ab′), an Fv, a scFv, a single domain antibody, a diabody, a bi-specific antibody, or a multi-specific antibody.

95. The binding agent of claim 84, wherein the binding agent further comprises a heavy chain constant region.

96. The binding agent of claim 95, wherein the heavy chain constant region is of the IgG isotype.

97. The binding agent of claim 96, wherein the heavy chain constant region is an IgG1 constant region.

98. The binding agent of claim 97, wherein the IgG1 constant region has the amino acid sequence set forth in SEQ ID NO:28.

99. The binding agent of claim 84, wherein the binding agent further comprises a light chain constant region.

100. The binding agent of claim 99, wherein the light chain constant region is of the kappa isotype.

101. The binding agent of claim 100, wherein the kappa isotype has the amino acid sequence set forth in SEQ ID NO:29.

102. The binding agent of claim 95, the heavy chain constant region further comprises L234A and L235A mutations, according to the EU numbering of Kabat.

103. A nucleic acid encoding the binding agent of claim 84.

104. A vector comprising the nucleic acid of claim 103.

105. A cell line comprising the nucleic acid of claim 103.

106. A conjugate comprising:
the binding agent of claim 84,
at least one linker attached to the binding agent; and
at least one drug attached to each linker.

107. The conjugate of claim 106, wherein each linker is attached to the binding agent via an interchain disulfide residue, a lysine residue, an engineered cysteine residue, a glycan, a modified glycan, an N-terminal residue of the binding agent or a polyhistidine peptide attached to the binding agent.

108. The conjugate of claim 106, wherein the average drug loading of the conjugate is from about 1 to about 16.

109. The conjugate of claim 108, wherein the average drug loading of the conjugate is from about 1 to about 8.

110. The conjugate of claim 109, wherein the average drug loading of the conjugate is 4 or 8.

111. The conjugate of claim 106, wherein each drug is selected from a cytotoxic agent, an immunomodulatory agent, a nucleic acid, a growth inhibitory agent, a PROTAC, a toxin and a radioactive isotope.

112. The conjugate of claim 111, wherein the drug is a cytotoxic agent.

113. The conjugate of claim 106, wherein the linker comprises mc-VC-PAB, CL2, CL2A or (Succinimid-3-yl-N)—(CH2)ₙ—C(═O)-Gly-Gly-Phe-Gly-NH—CH₂—O—CH₂—(C═O)—, wherein n=1 to 5.

114. A pharmaceutical composition comprising the binding agent of claim 84 and a pharmaceutically acceptable carrier.

115. A pharmaceutical composition comprising the conjugate of claim 106 and a pharmaceutically acceptable carrier.

116. A method of treating a CD70+ cancer, comprising administering to a subject in need thereof a therapeutically effective amount of the pharmaceutical composition of claim 114.

117. A method of treating a CD70+ cancer, comprising administering to a subject in need thereof a therapeutically effective amount of the pharmaceutical composition of claim 115.
